(19) United States
(12) Patent Application Publication
(10) Pub. No.: US 2015/0306232 A1
(43) Pub. Date: Oct. 29, 2015

(54) HYDROGELATORS COMPRISING D-AMINO ACIDS

(71) Applicant: BRANDEIS UNIVERSITY, Waltham, MA (US)

(72) Inventors: Bing XU, Newton, MA (US); Jiayang LI, Waltham, MA (US); Kuang YI, Waltham, MA (US)

(21) Appl. No.: 14/441,773

(22) PCT Filed: Nov. 8, 2013
(86) PCT No.: PCT/US13/69090

§ 371 Date: May 8, 2015

Related U.S. Application Data
(60) Provisional application No. 61/724,026, filed on Nov. 8, 2012.

(51) Int. Cl. A61K 47/42; A61K 9/00; A61K 31/192; C07K 5/107

(52) U.S. Cl. CPC A61K 47/42; C07K 5/1016; A61K 9/0014; A61K 31/192

(57) ABSTRACT

Described herein are compounds comprising an oligopeptide and a non-steroidal antiinflammatory agent. The compounds self-assemble into supramolecular hydrogels and can be used as topical treatments for inflammatory conditions, such as osteoarthritis. Also described herein are oligopeptides compounds made from D-amino acid residues that form supramolecular hydrogels. The compounds may be functionalized with active agents, such as anticancer therapeutic agents, antiinflammatory agents, or imaging agents, therefore providing new mechanisms for delivery of active agents.

### RELATED APPLICATIONS

This application claims the benefit of priority to U.S. Provisional Patent Application Ser. No. 61/724,026, filed Nov. 8, 2012, the contents of which are hereby incorporated by reference.

### GOVERNMENT SUPPORT

This invention was made with government support under R01 CA142746 awarded by the National Institutes of Health. The government has certain rights in the invention.

### BACKGROUND OF THE INVENTION

### Supramolecular Hydrogels

Supramolecular hydrogels, a type of hydrogels resulting from the self-assembly of small molecules (usually called “hydrogelators”) in water, have become an attractive choice of soft nanomaterials for a variety of applications, such as scaffolds for tissue engineering, carriers for drug delivery, biosensing, wound healing, ultrathin membranes, and new matrices for enzyme assays, antibacterial cell cultures, gel electrophoresis, and protein pull-down assays. Since hydrogelators only associate with each other through non-covalent interactions, supramolecular hydrogels are inherently biocompatible and biodegradable. These features make hydrogelators attractive candidates for self-delivery therapeutics, that is, when drug molecules themselves are hydrogelators. Self-delivery hydrogels based on supramolecular hydrogelators minimize several inherent shortcomings of more typical drug delivery systems, such as encapsulating therapeutic agents in functionalized or engineered biodegradable polymers for controlled release of drugs. Inherent shortcomings of encapsulated systems include inflammation, limited loading of drug molecules, and difficulties functionalizing the polymers with drug molecules.

Small peptides made of L-amino acid residues undergo a process referred to as enzymatic hydrogelation, such that the solution of a precursor of a hydrogelator, upon the addition of an enzyme, turns into a gel. Enzymatic hydrogelation has already been utilized in a wide range of applications, such as screening the inhibitors of enzymes, measuring enzyme activity, modulating biomineralization, typing bacteria, delivering drugs or proteins, stabilizing enzymes, and regulating the fate of cells. L-peptides, however, are susceptible to degradation catalyzed by various endogenous proteases; therefore, the usefulness of supramolecular hydrogels of L-peptides is limited when long-term biostability is required (such as in applications relating to controlled drug release, intracellular imaging, or other in vivo applications).

Therefore, there exists a need for hydrogel systems that undergo enzymatic hydrogelation to form hydrogels that are stable for a prolonged period inside cells or in vivo. When they include a therapeutic or imaging moiety, these hydrogels could be used for therapeutic or diagnostic purposes.

### Non-Steroidal Antiinflammatory Drugs

Non-steroidal antiinflammatory drugs (NSAIDs) are widely, systemically used drugs for the treatment of acute or chronic pain or inflammation, usually administered in high dosages. These high dosages can cause adverse gastrointestinal and renal effects when the drugs are inhibitors of COX-1, and are associated with cardiovascular risks when the drugs inhibit COX-2. Because of these adverse effects, the selectivity of NSAIDs must be modulated according to the therapeutic objectives. In addition, the known adverse side effects require that systemic use of NSAIDs for localized acute or chronic pain be minimized.

Diclofenac, a NSAID, has been formulated into a lotion for managing moderate osteoarthritis with promising results.

Therefore, there exists a need for compositions comprising NSAIDs as biostable, highly active topical agents for treating inflammation and relieving pain.

### SUMMARY OF THE INVENTION

In certain embodiments, the invention relates to a hydrogelator of Formula III

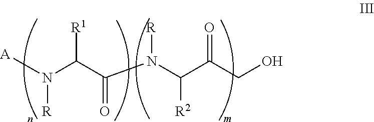

or a pharmaceutically acceptable salt thereof,
wherein, independently for each occurrence,

A is selected from the group consisting of

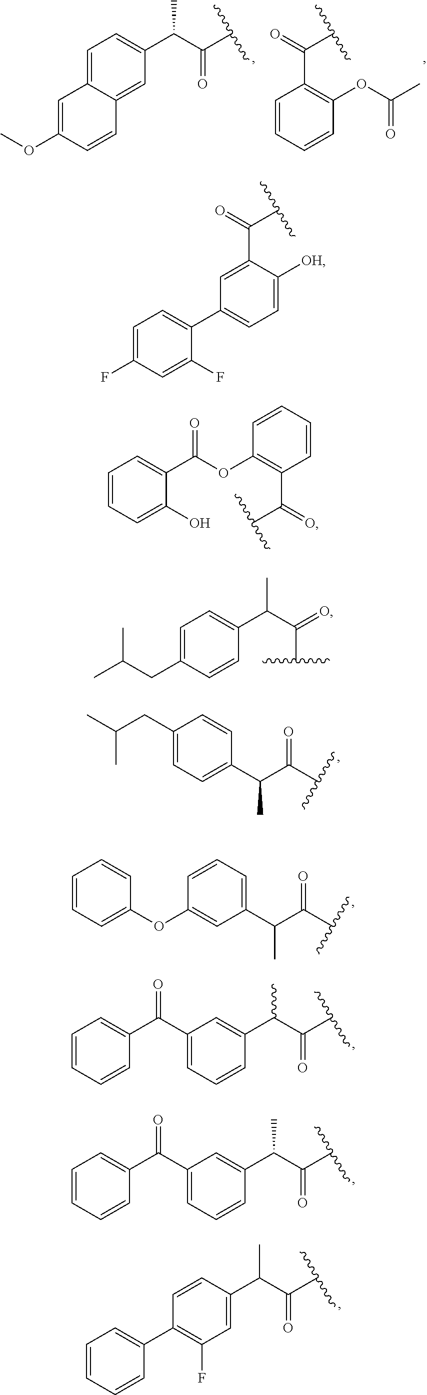

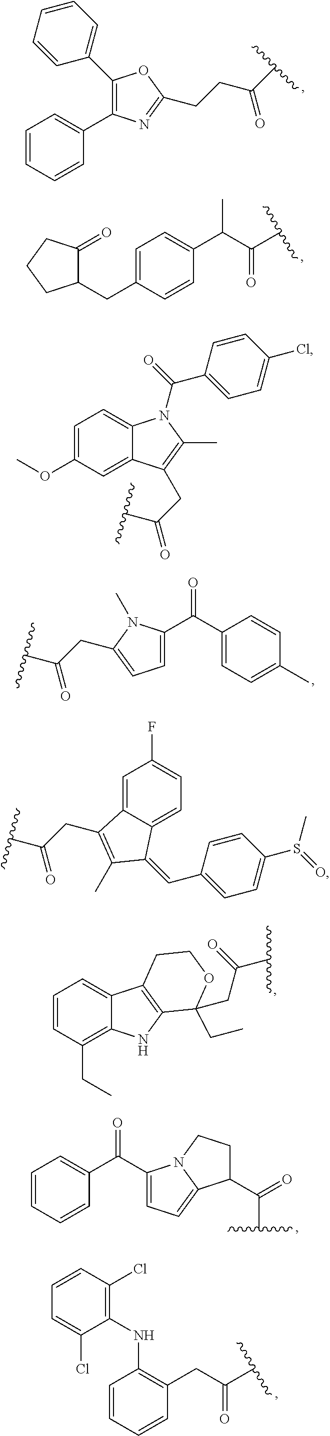

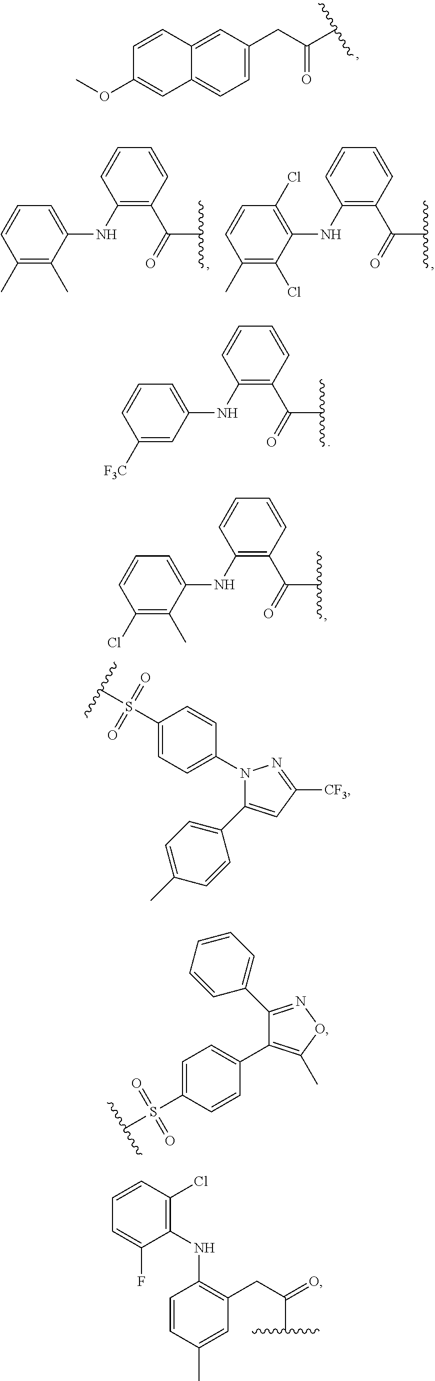

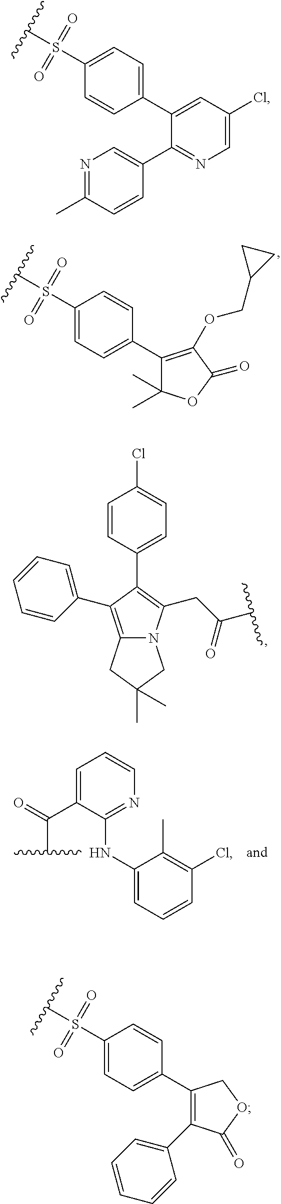

R is H or alkyl;

R¹ is aralkyl, heteroaralkyl, hydroxyaralkyl, or phosphorylated aralkyl;

R² is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, or HS-alkyl;

n is 1, 2, 3, or 4; and

m is 0, 1, 2, 3, or 4.

In certain embodiments, the invention relates to a hydrogelator of Formula IV

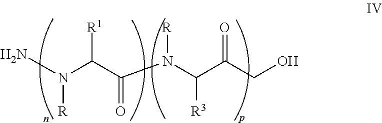

or a pharmaceutically acceptable salt thereof,
wherein, independently for each occurrence,

R is H or alkyl;

R¹ is aralkyl, heteroaralkyl, hydroxyaralkyl, or phosphorylated aralkyl;

R³ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or A-NR-alkyl, provided at least one instance of R³ is A-NR-alkyl;

n is 1, 2, 3, or 4;

p is 1, 2, 3, or 4; and

A is selected from the group consisting of

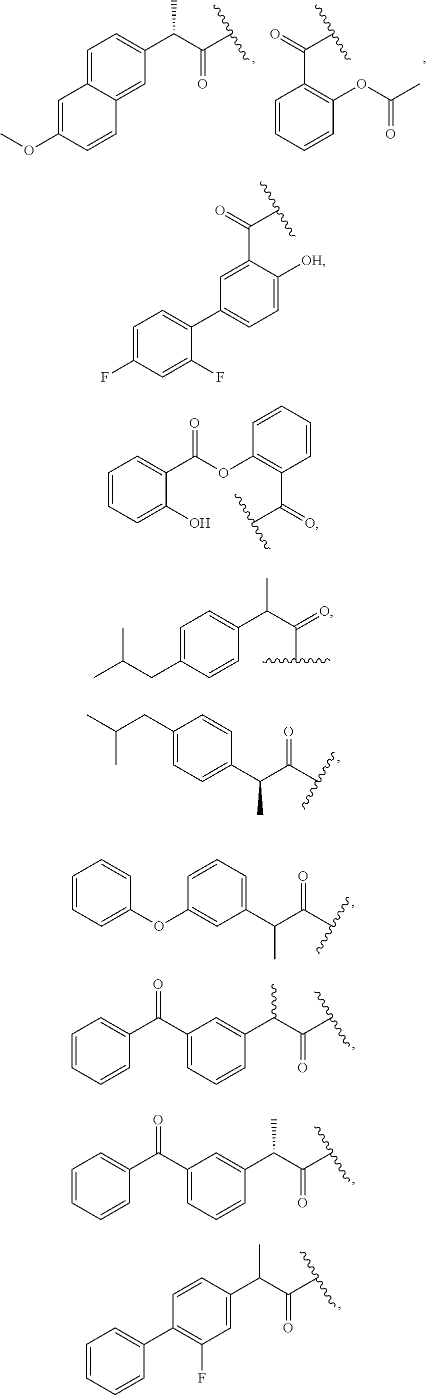

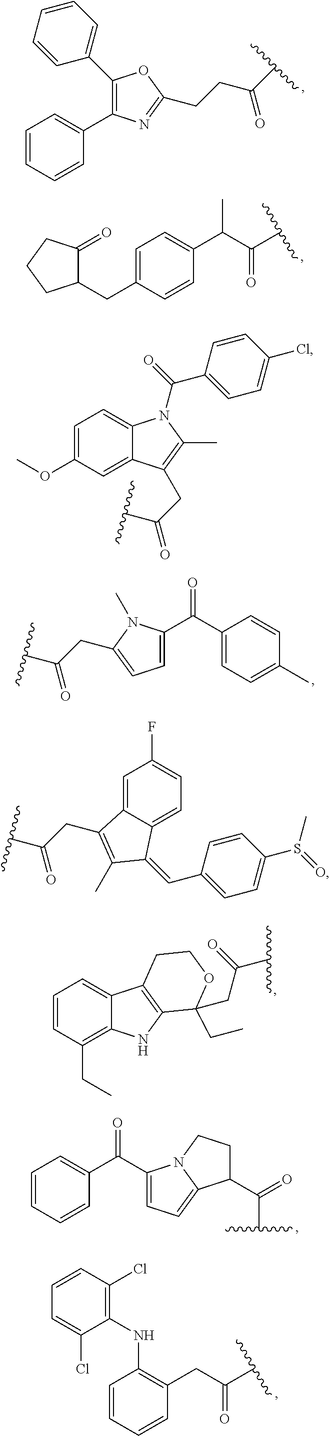

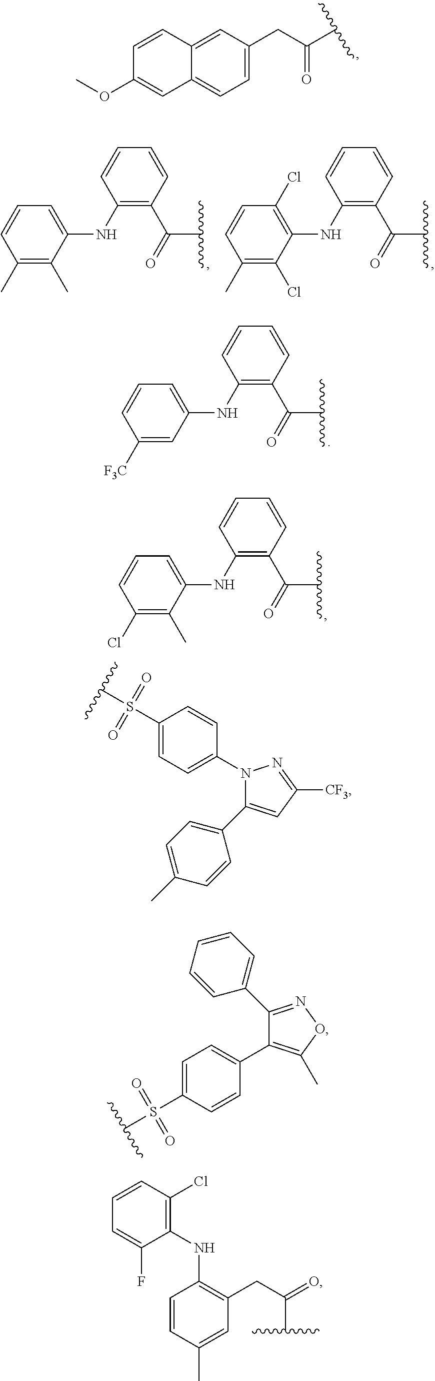

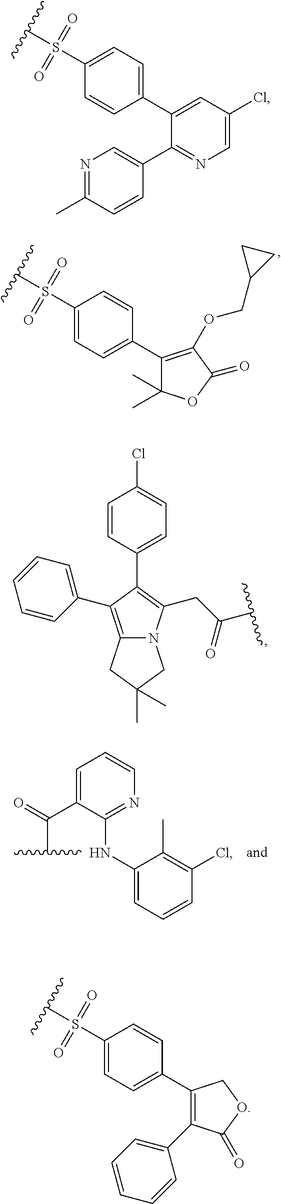

In certain embodiments, the invention relates to any one of the aforementioned hydrogelators, wherein each amino acid residue is in the D-configuration.

In certain embodiments, the invention relates to a hydrogelator selected from the group consisting of:

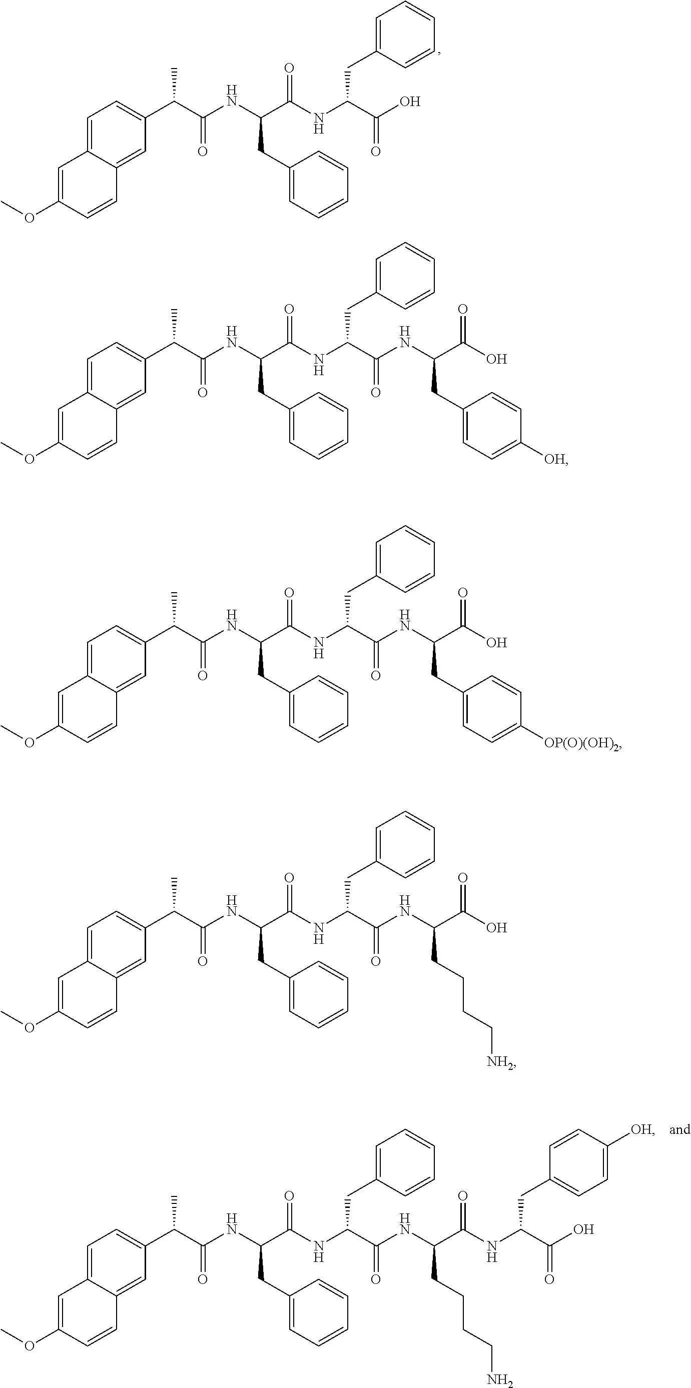

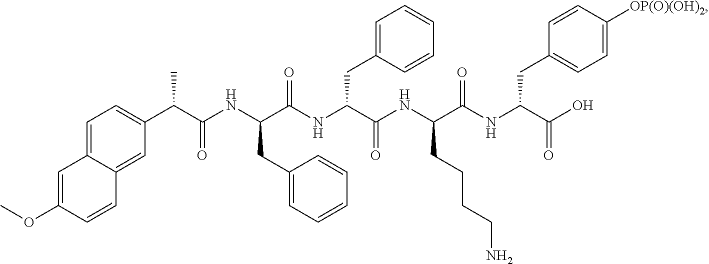

or a pharmaceutically acceptable salt thereof.

In certain embodiments, the invention relates to a hydrogelator selected from the group consisting of:

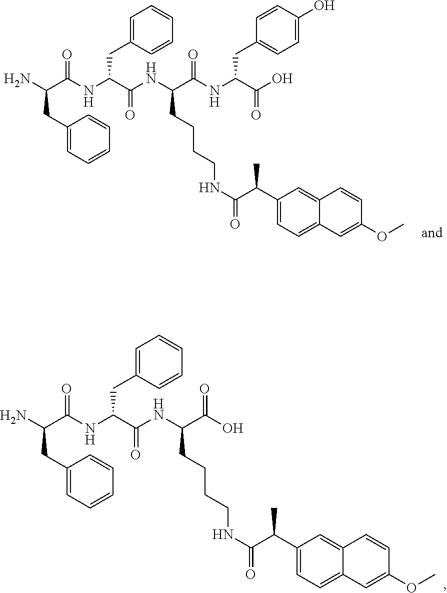 [SMILES: COc1ccc2cc([C@H](C)C(=O)NCCCC[C@@H](NC(=O)[C@@H](Cc3ccccc3)NC(=O)[C@H](N)Cc3ccccc3)C(=O)N[C@H](Cc3ccc(O)cc3)C(=O)O)ccc2c1 ; COc1ccc2cc([C@H](C)C(=O)NCCCC[C@@H](NC(=O)[C@@H](Cc3ccccc3)NC(=O)[C@H](N)Cc3ccccc3)C(=O)O)ccc2c1]

or a pharmaceutically acceptable salt thereof.

In certain embodiments, the invention relates to a supramolecular structure comprising, consisting essentially of, or consisting of a plurality of any one of the aforementioned hydrogelators.

In certain embodiments, the invention relates to a hydrogel, comprising, consisting essentially of, or consisting of a plurality of any one of the aforementioned hydrogelators; and water.

In certain embodiments, the invention relates to a method of treating an inflammatory condition, comprising

administering to a subject in need thereof a therapeutically effective amount of any one of the aforementioned hydrogelators, any one of the aforementioned supramolecular structures, or any one of the aforementioned hydrogels.

In certain embodiments, the invention relates to a hydrogelator of Formula I

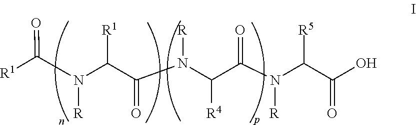

or a pharmaceutically acceptable salt thereof,
wherein, independently for each occurrence,

R is H or alkyl;

R¹ is aralkyl, heteroaralkyl, hydroxyaralkyl, or phosphorylated aralkyl;

R⁴ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or substituted aminoalkyl;

R⁵ is hydroxyaralkyl or phosphorylated aralkyl;

n is 1, 2, 3, or 4; and

p is 1, 2, 3, or 4,

provided that each amino acid residue of the hydrogelator is in the D-configuration.

In certain embodiments, the invention relates to a hydrogelator of Formula II

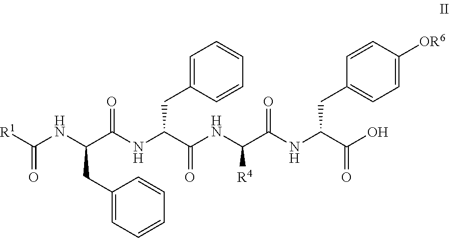

or a pharmaceutically acceptable salt thereof,
wherein, independently for each occurrence,

R¹ is aralkyl, heteroaralkyl, hydroxyaralkyl, or phosphorylated aralkyl;

R⁴ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or substituted aminoalkyl; and

R⁶ is H or P(O)(OH)₂.

In certain embodiments, the invention relates to a hydrogelator selected from the group consisting of:

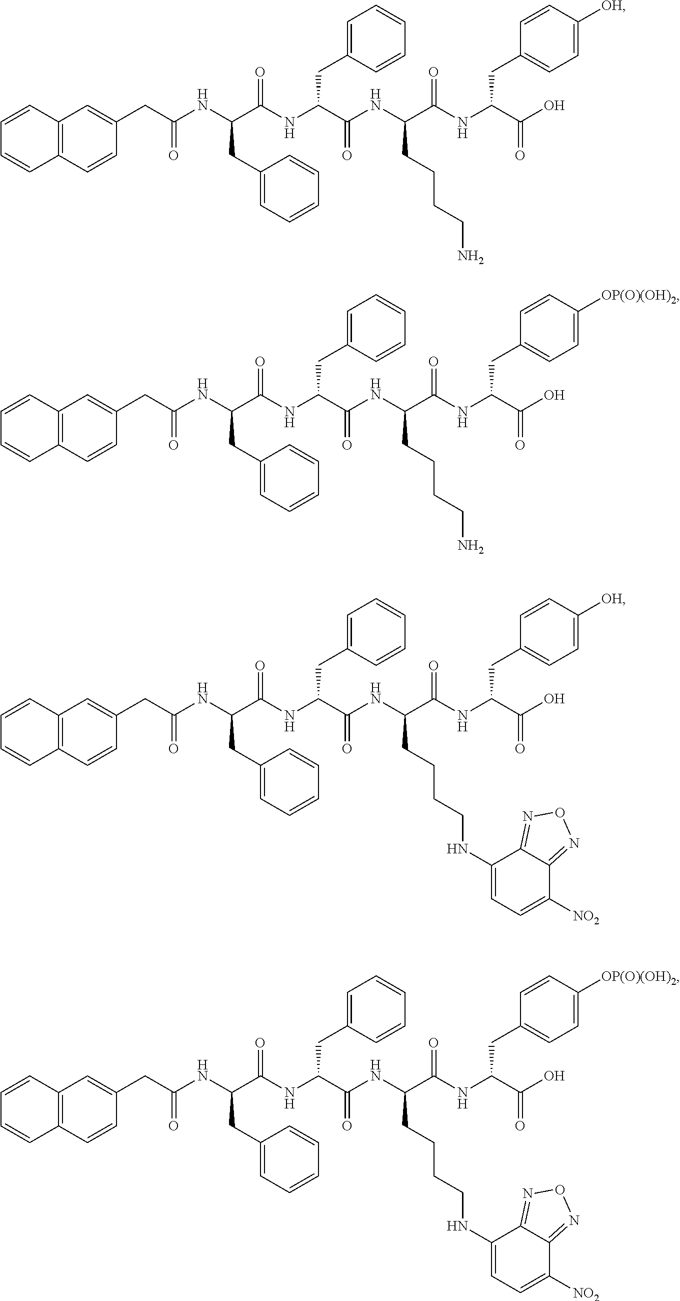

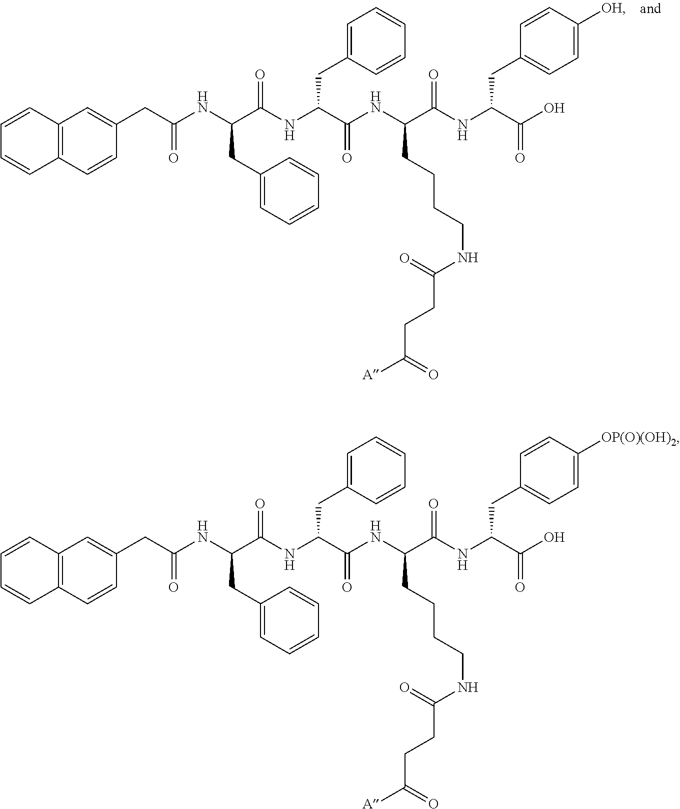

wherein A″ is

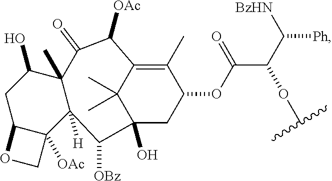

or a pharmaceutically acceptable salt thereof.

In certain embodiments, the invention relates to a supramolecular structure comprising, consisting essentially of, or consisting of a plurality of any one of the aforementioned hydrogelators.

In certain embodiments, the invention relates to a hydrogel, comprising, consisting essentially of, or consisting of a plurality of any one of the aforementioned hydrogelators; and water.

In certain embodiments, the invention relates to a method of treating cancer, tumors, malignancies, neoplasms, or other dysproliferative diseases, comprising

administering to a subject in need thereof a therapeutically effective amount of any one of the aforementioned hydrogelators, any one of the aforementioned supramolecular structures, or any one of the aforementioned hydrogels, wherein the hydrogelator comprises a radical of an active agent; and the active agent is an anticancer agent.

In certain embodiments, the invention relates to a method of in vivo imaging, comprising

administering to a subject in need thereof a diagnostically effective amount of any one of the aforementioned hydrogelators, any one of the aforementioned supramolecular structures, or any one of the aforementioned hydrogels, wherein the hydrogelator comprises a radical of an active agent; and the active agent is a fluorophore.

### BRIEF DESCRIPTION OF THE FIGURES

FIG. 1 depicts the binding of (A) Npx and (B) 1 with COX-2 enzyme (the ligands as CPK model and the COX-2 as ribbons).

FIG. 2 depicts the TEM images of the hydrogels of (A) 1 (pH 4.0); (B) 2 (pH 7.6); (C) 3 (pH 7.6); (D) 4 (pH 7.6); (E) 5 (pH 7.0); (F) 6 (pH 7.0) (inset: optical images). Hydrogels of 2 and 4 are made by the addition of 1.0 U/mL alkaline phosphatase to the solutions of 2P and 4P. All hydrogels have 0.8 wt % concentrations (f=D-Phe, k=D-Lys, y=D-Try). The scale bar is 100 nm.

FIG. 3 depicts (A) The IC₅₀ values of the Npx based hydrogelators for inhibiting COX enzyme (the selectivity, defined as the IC₅₀ ratio of COX-1 and COX-2, is labeled on the top of the bars) (left bar=inhibition for COX-1; right bar=inhibition for COX-2). (B) The release profiles of the Npx based hydrogelators from hydrogels of 1, 2, 3, 4, 5, and 6.

FIG. 4 depicts the structures of the hydrogelators consisting of D-amino acids and naproxen (Npx).

FIG. 5 tabulates the rheological properties and TEM characteristics of the hydrogels of the conjugates of D-amino acids and naproxen. ^{a}The value is taken at frequency equals 6.28 rad/s.

FIG. 6 depicts (left) IC₅₀ values of the Npx based hydrogelators for inhibiting COX enzyme (the selectivity, S, defined as the IC₅₀ ratio of COX-1 and COX-2, is labeled) (top bar=inhibition for COX-1; bottom bar=inhibition for COX-2); (top right) a TEM image of a hydrogelator of the invention; and (bottom right) an optical image of a hydrogelator of the invention.

FIG. 7 tabulates the IC₅₀ values for naproxen based hydrogelators inhibiting COX-1 and COX-2 enzymes. ^{a}The selectivity for COX-2 enzyme is calculated by the equation: IC₅₀ of COX-1/IC₅₀ of COX-2.

FIG. 8 depicts optical images and TEM images of hydrogels formed by using ALP (1.0 U/mL) to treat 0.4 wt % of (A) 11a and (B) 11b at pH 7.6. (C) The strain sweep and (D) the frequency sweep of the hydrogels 12a (squares) and 12b (circles).

FIG. 9 depicts (A) The optical image and TEM image of hydrogel formed by 0.4 wt % of 14b at pH 7.4 upon the catalysis of ALP (20.0 U/mL). (B) The fluorescent confocal microscope image of a HeLa cell incubated with 500 μM of 14b in PBS buffer (scale bar is 10 μm). The fluorescent confocal microscope images of HeLa cells incubated with 500 μM of 14b without (C) or with (D) the PTP1B inhibitor (25 μM) (scale bar is 50 μm).

FIG. 10 depicts (A) optical and TEM images of hydrogel formed by 1.8 wt % of 20b at pH 7.4 with the catalysis of ALP (1 U/mL) with scale of 100 nm; (B) The IC₅₀ values of 16 (left bar), 19b (middle bar), and 20b (right bar) incubated with HeLa cells after 72 h; (C) The relative tumor sizes and (D) relative weights of mice treated with 16 (squares), 20a (light grey triangles), and 20b (dark grey triangles, upside down) for in vivo tests.

FIG. 11 depicts optical images of hydrogels (between a pair of crossed polarizers) formed by (A) 0.4 wt % of 12a, (B) 1.0 wt % of 12a, (C) 0.4 wt % of 12b, (B) 1.0 wt % of 12b. The light spots are mainly coming from the bubbles and dusts, which also could be observed without polarized light.

FIG. 12 depicts TEM images of (A) 0.4 wt % of 12a and (B) 0.4 wt % of 12b with scale bar indicating 20 nm.

FIG. 13 depicts the strain (A) and frequency (B) dependence of dynamic storage modulus G′ (solid) and loss modulus G″ (hollow) of the gels formed by 12b upon the treatment of 1.0 U/mL enzyme at pH 7.6. The values of (C) critical strains and (D) storage moduli at frequency of 6.28 rad/s vs. concentrations of hydrogels of 12b.

FIG. 14 depicts the strain (A) and frequency (B) dependence of dynamic storage modulus G′ (solid) and loss modulus G″ (hollow) of the gels formed by 0.4 wt % of 15b upon the treatment of 20.0 U/mL enzyme at pH 7.4; The strain (C) and frequency (D) dependence of dynamic storage modulus G′ (solid) and loss modulus G″ (hollow) of the gels formed by 1.8 wt % of 20b at pH 7.4.

FIG. 15 depicts MTT assays for (A) 19b, (B) 20b, (C) 16, (D) 14b, and (E) 11b on HeLa cells for 72 hours. In (A), (B), and (C), from left to right, the bars indicate 1 nM, 2 nM, 5 nM, 10 nM, 20 nM, 50 nM, 100 nM, and 200 nM concentrations. In (D), from left to right, the bars indicate 20 μM, 50 μM, 100 μM, 200 μM, and 500 μM concentrations. In E, from left to right, the bars indicate 125 μM, 250 μM, and 500 μM concentrations.

FIG. 16 depicts the time course of HeLa cells incubated with 500 μM of 14b and 14a without (−) or with (+) the PTP1B inhibitor (25 μM) (scale bar is 50 μm) within 7.5 minutes (t=2 min, 3 min, 4 min, and 5 min are 14b; t=7.5 min is 14a).

FIG. 17 depicts the time-dependent course of digestion of 11a (squares) and 11b (circles) by proteinase K.

FIG. 18 depicts the COX-1 enzyme activity curves for (A) Npx, (C) D-version hydrogelators 1, 2, 3, 4, 5, and 6, and (E) L-version hydrogelators L-1, L-2, L-3, and L-4; and the COX-2 enzyme activity curves for (B) Npx, and (D) D-version hydrogelators 1, 2, 3, 4, 5, and 6, and (F) L-version hydrogelators L-1, L-2, L-3, and L-4.

FIG. 19 depicts IC₅₀ values of exemplary Npx containing hydrogelators. Left bar=inhibition for COX-1; right bar=inhibition for COX-2.

FIG. 20 depicts the cytotoxicity of (A) 1, (B) 2, (C) 3, (D) 4, (E) 5, (F) 6 and (G) Npx treated with HeLa cells for 3 days; (H) the activity curves of HeLa cell after 72 hours. For figures A-H, left bar=20 μM, second left bar=50 μM, middle bar=100 μM, second right bar=200 μM, right bar=500 μM.

FIG. 21 depicts the strain (A) and frequency (B) dependence of dynamic storage modulus G′ (solid) and loss modulus G″ (hollow) of Npx containing hydrogels of 1, 2, 3, 4, 5, and 6.

FIG. 22 depicts the binding of the phosphate precursors to the active site of an ALP (presented as solid ribbons). (A) L-peptide based precursor (11a) and (B) D-peptide based precursor (11b) binding to the phosphatase. (C) Top view and (D) side view of 11a and 11b in the active site.

FIG. 23 depicts a schematic representation of a synthetic route of the precursor of the NBD or Taxol-containing hydrogelator based on a D-peptide.

FIG. 24 depicts TEM images of hydrogels formed by using ALP (1.0 U/mL) to treat 11b at pH 7.6 and concentrations of (A) 0.4 wt %, (B) 0.6 wt % (C) 0.8 wt %, and (D) 1.0 wt %. Inset: optical images. Scale bar is 100 nm.

FIG. 25 tabulates rheological properties and TEM characteristics of hydrogels of 12a, 12b, 15b, and 20b. ^{a}The value is taken at frequency equals 6.28 rad/s. ^{b}The hydrogel is formed at pH 7.4, while others are formed at pH 7.6.

FIG. 26 depicts ³¹P NMR spectra showing the conversion of 1.0 wt % of (A) 11a and (B) 11b catalyzed by the phosphatase (0.02 U/mL) at pH 7.6 at 3 minutes and 4, 12, 24, and 48 h; The time dependent rheology study of 1.0 wt % of (C) 11a and (D) 11b catalyzed by the phosphatase (0.02 U/mL) at pH 7.6.

### DETAILED DESCRIPTION OF THE INVENTION

### Design, Synthesis, and Discussion of D-Amino Acid-Containing Hydrogelators

In certain embodiments, the invention relates to the use of D-amino acids to replace L-amino acids. In certain embodiments, the oligopeptides made from D-amino acids are protease resistant. In addition, D-peptides may play a special role in defense mechanisms as “alien” agents from other organisms, act as potent inhibitors to inhibit HIV-1 entry, inhibit tumor cell migration, reduce adverse drug reactions (ADRs), control the formation and disassembly of bacteria biofilms, bind to DNA, form β-sheets, and dissociate Alzheimer's amyloid to reduce the cytotoxicity induced by amyloid.

In certain embodiments, the invention relates to oligopeptides made from D-amino acid residues that undergo enzymatic dephosphorylation to form a hydrogel. In certain embodiments, the invention relates to oligopeptides functionalized with therapeutic agents or fluorophores, which form biostable or biocompatible hydrogels/nanofibers that may find applications in intratumoral chemotherapy or intracellular imaging.

Molecular Design.

2-(naphthalen-2-yl)acetic-Phe-Phe (NapFF) is an excellent motif for enabling self-assembly and hydrogelation due to its strong supramolecular interactions arising from aromatic-aromatic interactions and hydrogen bonds among the molecules. Since lysine (K) possesses an s-amine site for the attachment of biofunctional molecules on the side chain, and tyrosine phosphate (Y(p)) offers a handle for enzyme instructed hydrogelation, the incorporation of K and Y(p) with NapFF provides a versatile hydrogelator precursor NapFFKY(p) (11a), which undergoes enzymatic hydrogelation. D-amino acids, such as D-Phe (f), D-Lys (k), and D-Tyr phosphate (y(p)), may be used to replace the corresponding L-amino acids for making a more biostable precursor Napffky(p) (11b). To evaluate whether the dephosphorylation of D-tyrosine phosphate (y(p)) from the D-peptide by the phosphatase still would be possible, we first examined the binding of the tyrosine phosphate on 11a or 11b with ALP according to the crystal structure of ALP. With the phosphate groups being anchored to the active site of ALP (see FIG. 22), the structures of the phosphatase that binds with L-peptide/D-peptide based precursors 11a and 11b are shown in FIG. 22A and FIG. 22B, respectively. Although there are stereochemical differences between 11a and 11b, the phosphate groups appear to be able to bind the same active site without any hindrance. According to the top view (FIG. 22C), the opening in the structure of ALP is large enough to accommodate either 11a or 11b. Similarly, the side view (FIG. 22D) clearly indicates that the phosphate groups on 11a or 11b are able to bind the active site of ALP. Thus, the enzymatic hydrogelation of 11b was investigated, and compared with that of 11a. The rate of formation, morphology, and viscoelastic properties of the corresponding hydrogels were compared.

To explore the biological and biomedical applications of 11b, we attached small functional molecules, such as 4-nitro-2,1,3-benzoxadiazole (NBD), a fluorophore used in cell imaging, and Taxol, a clinically-used anti-cancer drug, to 11b. Gao, Y.; et al. Nat. Commun. 2012, 3, 1033; and Gao, Y.; et al. J. Am. Chem. Soc. 2009, 131, 13576.

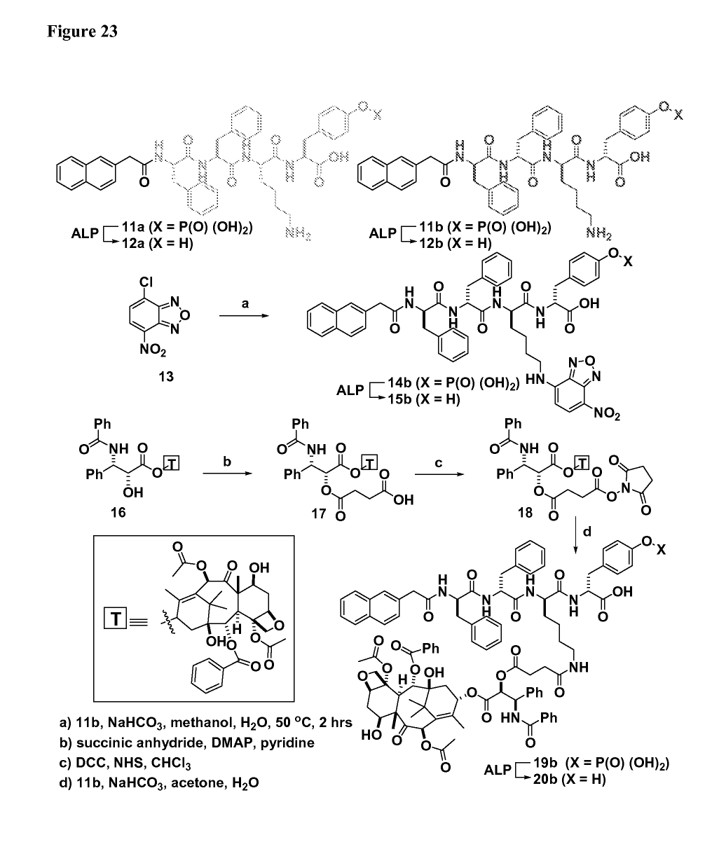

Synthesis. FIG. 23 shows the chemical structures of precursors 11a and 11b. Utilizing Fmoc-protected D-amino acids, we prepared 11b by standard solid phase synthesis with 2-chlorotrityl chloride resin (100˜200 mesh and 0.3˜0.8 mmol/g), followed by HPLC purification. We conjugated NBD group at the side chain of lysine to afford the precursor Napffk(NBD)y(p) (14b). As shown in FIG. 23, we dissolved 7-chloro-4-nitro-2,1,3-benzoxadiazole (NBD-Cl) (13) in methanol, followed by adding the basic aqueous solution of 11b (pH 9). The reaction of the mixed solution at 50° C. for 2 hours yields 14b as red precipitate after work-up and purification by reverse phase HPLC.

Using a similar approach, we obtained the conjugate of Taxol and 11b. As shown in FIG. 23, we added succinic anhydride and 4-dimethylamino-pyridine (DMAP) into the clear solution of Taxol in pyridine. After stirring the mixture at room temperature overnight, we extracted the solution with dichloromethane (DCM) and obtain Taxol-succinic acid (17). The conjugation of 17 and N-hydroxysuccinimide (NHS) with the aid of N,N′-dicyclohexylcarbodiimide (DCC) affords Taxol-succinic-NHS ester (18). Purified with column chromatography, we collected pure 18 and re-dissolved it with acetone. Then we added the acetone solution into a basic aqueous solution (pH 8.5) of 15b, which reacted for 24 hours. After working up the reaction and using reverse phase HPLC for the purification, we obtained compound 19b as the conjugate of Taxol and 11b.

Hydrogelation of the D-Peptidic Hydrogelator (12b).

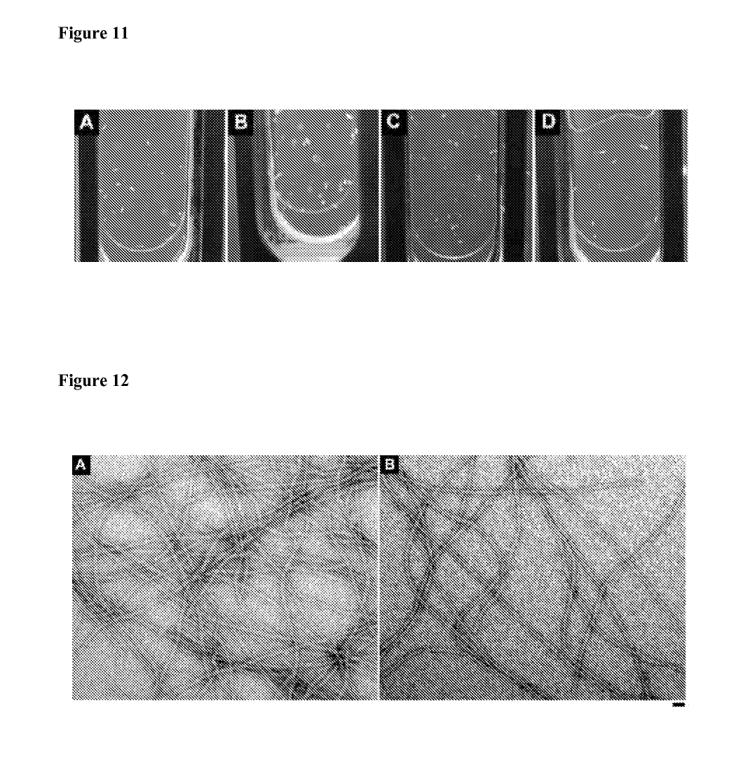

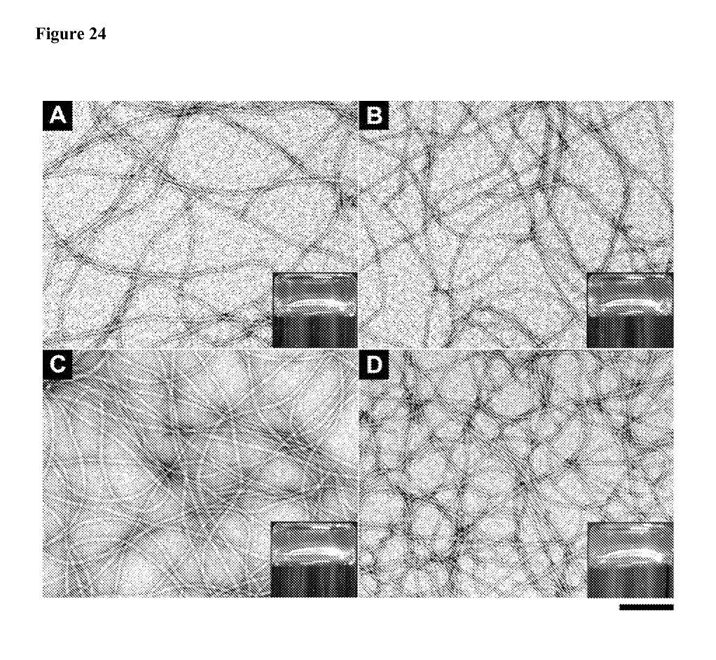

To investigate the enzymatic hydrogelation of the D-peptidic precursor 11b, we prepared a series of hydrogels formed by using ALP to treat 11b at different concentrations. After dissolving 1.0, 2.0, 3.0, 4.0, and 5.0 mg of 11b in 0.5 mL of water (pH 7.6), respectively, we obtained clear solutions of 11b with different concentrations. The treatment of the solutions of 11b with ALP (1.0 U/mL) afforded the molecules of hydrogelator 12b, which are less soluble than 11b and thus self-assemble in water to form hydrogels when the concentrations of 12b are sufficient. As shown in FIG. 24, except the solution of 0.2 wt % of 11b, solutions of 11b with the concentration of 0.4, 0.6, 0.8, or 1.0 wt % form a stable transparent hydrogel within 24 h after the addition of 1.0 U/mL ALP into the solutions. Furthermore, as shown of the optical images in FIG. 24, the higher concentration of the solutions of 11b gives the less transparent hydrogels of 12b, which also exhibit little birefringence (FIG. 11), indicating that excess overlapping of the nanofibers to form large domains in the hydrogels of 12b cause the scattering of the light.

Being complementary to the optical images that serve as a simple way for proving the macroscopic phase transition (i.e., hydrogelation) triggered by the addition of ALP, transmission electron microscopy (TEM) images reveal the ordered nanostructures (e.g., nanofibers), formed by the self-assembly of the hydrogelators, that lead to hydrogelation. As shown in FIG. 24, the TEM images of all the hydrogels, which consist of different concentrations of 12b, exhibit long, flexible, and uniform nanofibers that entangle to form stable networks. With the increase of the concentrations of hydrogelator 12b (0.4, 0.6, 0.8, and 1.0 wt %), the densities of the nanofibers in the hydrogels increase, but the widths of the nanofibers in the hydrogels remain similar (around 9±2 nm). These results indicate that concentration of the hydrogelator 12b hardly affect the self-assembling process controlled by the enzymatic hydrogelation so that the nanofibers exhibit similar morphology regardless the concentrations of the precursor solutions. The concentrations of the hydrogelators correlate well with the densities of nanofibers, which should match with the viscoelastic behaviors of the hydrogels.

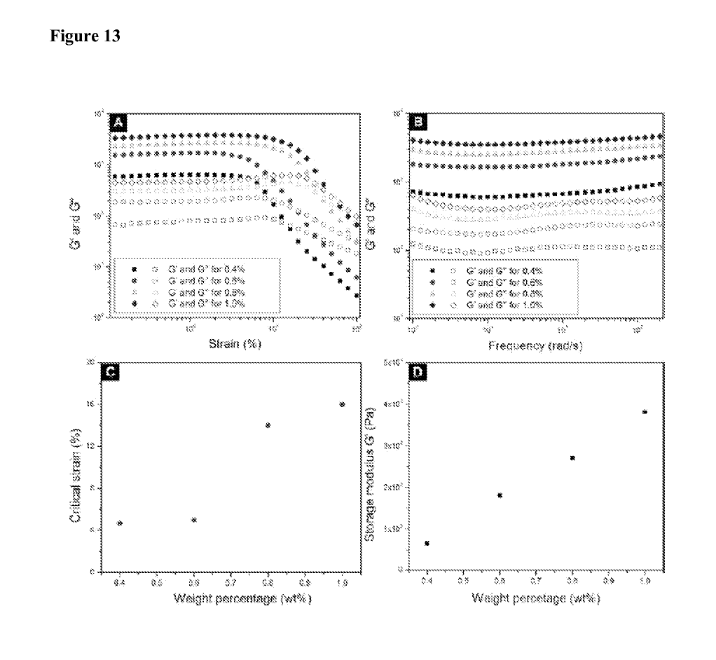

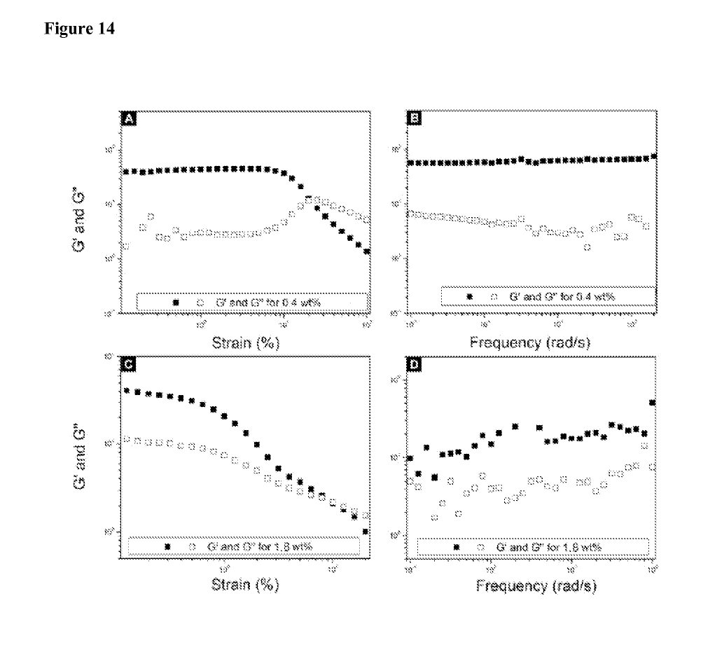

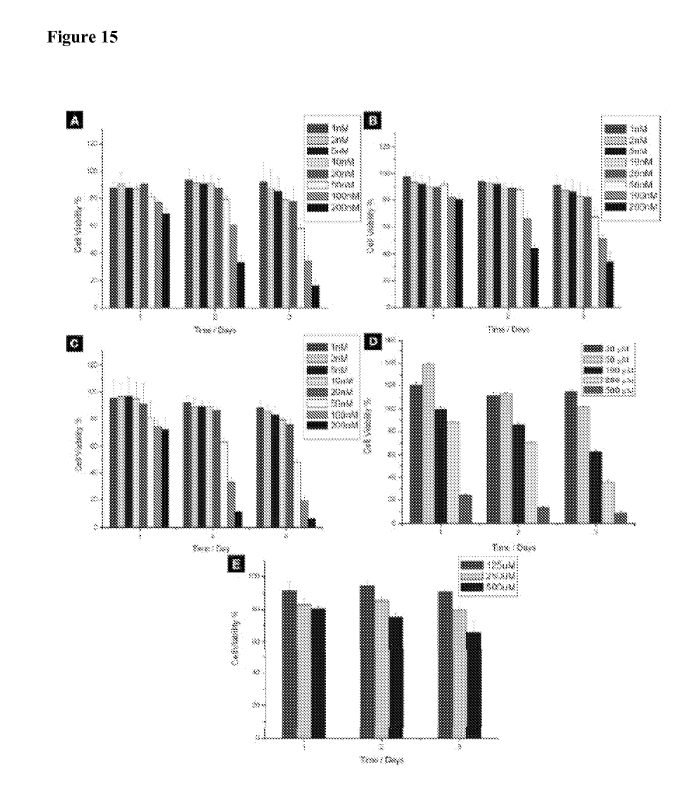

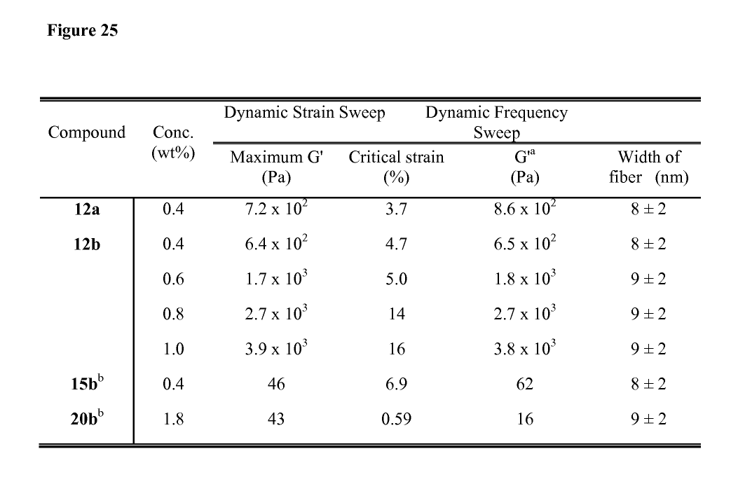

The oscillatory rheological measurement of the hydrogels of 12b agrees with the density of the nanofibers in the hydrogels. The dynamic strain sweep, under constant oscillation frequencies and various oscillation strains, indicates that the storage moduli (G′s) of all these hydrogels are independent to strain until their critical strains reach, and G′s start to decrease drastically due to the breakdown of the networks of the hydrogels. After obtaining the maximum G′s of the hydrogels in dynamic strain sweep, we measure the frequency dependence of their storage moduli (G′s) and loss moduli (G″s) using dynamic frequency sweep at constant oscillation stain (the strain for maximum G′s) and temperature (25° C.) but varying oscillation frequency (0.1˜200 rad/s). All the hydrogels of 12b exhibit viscoelastic properties of solid-like materials, evidenced by that the values of their G′s are significant higher (more than five times) than those of their G″s and are independent of the frequency during dynamic frequency sweep (FIG. 13). As listed in FIG. 25, the hydrogels of 12b at the concentrations of 0.4, 0.6, 0.8, and 1.0 wt % exhibit strains of 4.7%, 5.0%, 14%, and 16%, respectively. In addition, their values of G′ (at the frequency of 6.28 rad/s) in dynamic frequency sweep are 6.5×10² Pa, 1.8×10³ Pa, 2.7×10³ Pa, and 3.8×10³ Pa. While the critical strains of the resulting hydrogels of 12b show little correlation with the concentrations of the hydrogelators (FIGS. 13C and 13D), the storage moduli of hydrogels of 12b increase with the concentrations of 12b. This result agrees with that more physical cross-linking of the nanofibers at high concentrations of the hydrogelators.

The Comparisons of L and D Enantiomers of the Precursors and Hydrogelators.

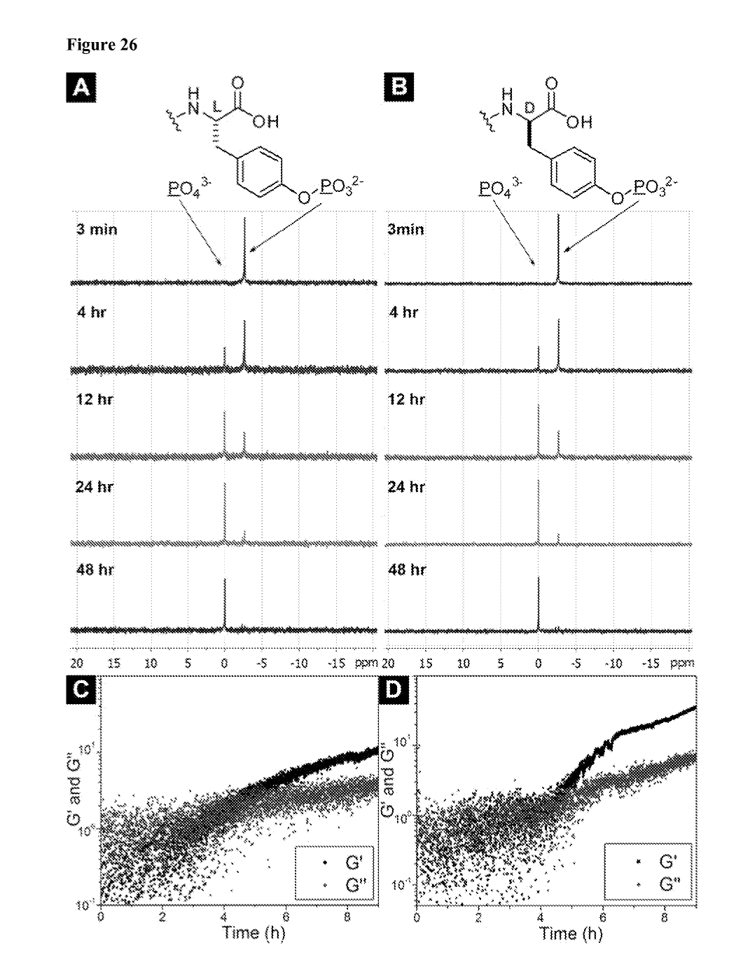

To evaluate the rate of enzymatic hydrogelation process, we used ³¹P NMR and rheology to study the transformation of the precursors 11a and 11b upon the treatment of ALP (FIG. 26). We first dissolved 10 mg of 11a and 11b into 1.0 mL of water at pH 7.6, respectively, to afford clear solutions with concentrations of 1.0 wt %. Once adding 0.02 U/mL of alkaline phosphates, we immediately monitored the solutions of 11a and 11b by ³¹P NMR and oscillatory rheology at 25° C. The ³¹P NMR spectra at 3 min, 1 h, 2 h, 4 h, 6 h, 8 h, 10 h, 12 h, 14 h, 18 h, 24 h, and 48 h indicate that the phosphate groups on the L-tyrosine of 11a and D-tyrosine 11b (δ=−2.7) become free phosphates (δ=0.0) at almost same rate, and dephosphorylation finishes after 48 hours. This result suggests that the precursors 11a and 11b undergo dephosphorylation with similar rates upon being treated with ALP. FIGS. 26C and 26D display the time dependent rheology studies of 11a and 11b. At the beginning, values of G″ are higher than the values of G′ for the solutions of 11a and 11b, indicating both of them are fluids. However, as 11a and 11b slowly turn into hydrogelators 12a and 12b by enzymatic dephosphorylation, the solutions start to form solid-like hydrogels with values of G′ become higher than those of G″. The gelation points for 12a and 12b, at where G′s intersect with G″s, are both achieved around 5 hours after the addition of enzyme. This result, together with the ³¹P NMR experiment, suggests that the chirality of 11a and 11b exhibits almost the same influence on the enzymatic hydrogelation catalyzed by ALP. The oscillatory shear during rheological measurement may accelerates enzymatic dephosphorylation so that the gelation points reach at the time (5 hours) much shorter than the time for completely dephosphorylation during the NMR experiment (48 hours).

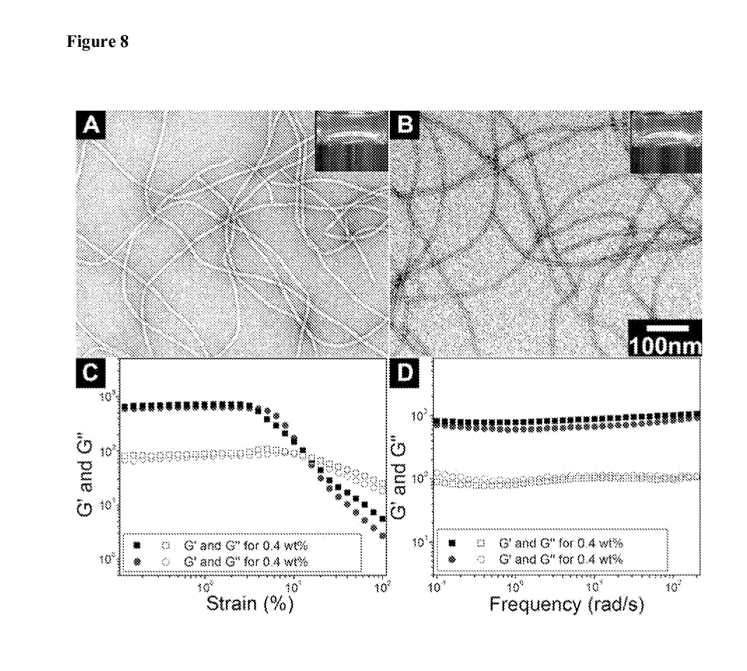

After comparing the rate of the dephosphorylation of the L- and D-enantiomeric precursors (11a and 11b), we examined the morphology of the microstructures and viscoelastic properties of the corresponding hydrogels (12a and 12b). By sonication, we dissolve 2.0 mg of 11a or 11b into 0.5 mL of water at pH 7.6 to afford a clear solution. The addition of 1.0 U/mL of ALP into the solution of 11a or 11b turns the hydrogelator precursor to its corresponding hydrogelator, 12a or 12b, which results in a transparent hydrogel (0.4 wt %) within 24 hours. As shown in FIGS. 8A and 8B, both hydrogelators 12a and 12b self-assemble to form long, flexible, and uniform nanofibers with average width around 8±2 nm, which entangle to develop physically cross-linked networks and to afford stable hydrogels. The similarity of the nanofibers in these two hydrogels indicates that chirality of 12a and 12b has little influence on the morphology of their nanofibers. Oscillatory rheology of the hydrogels of 12a and 12b indicates that both hydrogels behave as solid-like materials that have storage moduli (G′) to be significantly higher than loss moduli (G″) and exhibit weak frequency dependence in dynamic frequency sweep (FIGS. 8C and 8D). As shown in FIG. 25, hydrogels of 12a and 12b have critical strains of 3.7% and 4.7% during the dynamic strain sweep, and their values of G′ (at the frequency of 6.28 rad/s) in dynamic frequency sweep are 8.6×10² Pa and 6.5×10² Pa, respectively. These results suggest that the chirality of these two hydrogelators causes negligible differences on the viscoelastic properties of the corresponding hydrogels.

The Application of the D-Enantiomer Hydrogelator (11b) for Potential Intracellular Imaging.

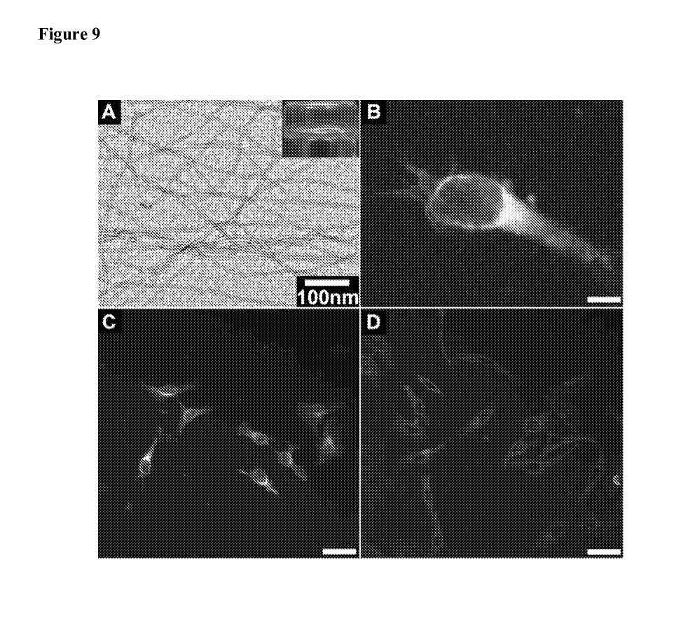

According to the molecular design, the attachment of functional molecules to 11b broadens the scope of the applications of supramolecular hydrogelators in cells or in vivo. We first examined the feasibility and characteristic of the use 14b for imaging intracellular self-assembly of D-peptidic hydrogelators. After dissolving 2.0 mg of 14b into 0.5 mL of water at pH 7.4, we treated the clear orange solution with 20.0 U/mL of ALP, which turns 14b into the fluorescent hydrogelator 15b. The self-assembly of 15b affords a transparent orange hydrogel (FIG. 9A, inset) that is stable over weeks. The TEM image of hydrogel of 15b exhibits long and uniform nanofibers with average width of 8±2 nm that entangle to afford stable network (FIG. 9A). The unassociated molecules of NBD containing hydrogelators in aqueous solutions exhibit little fluorescence unless they aggregate to form nanofibers. This important feature makes NBD containing hydrogelator be a useful candidate for imaging molecular self-assembly inside cells.

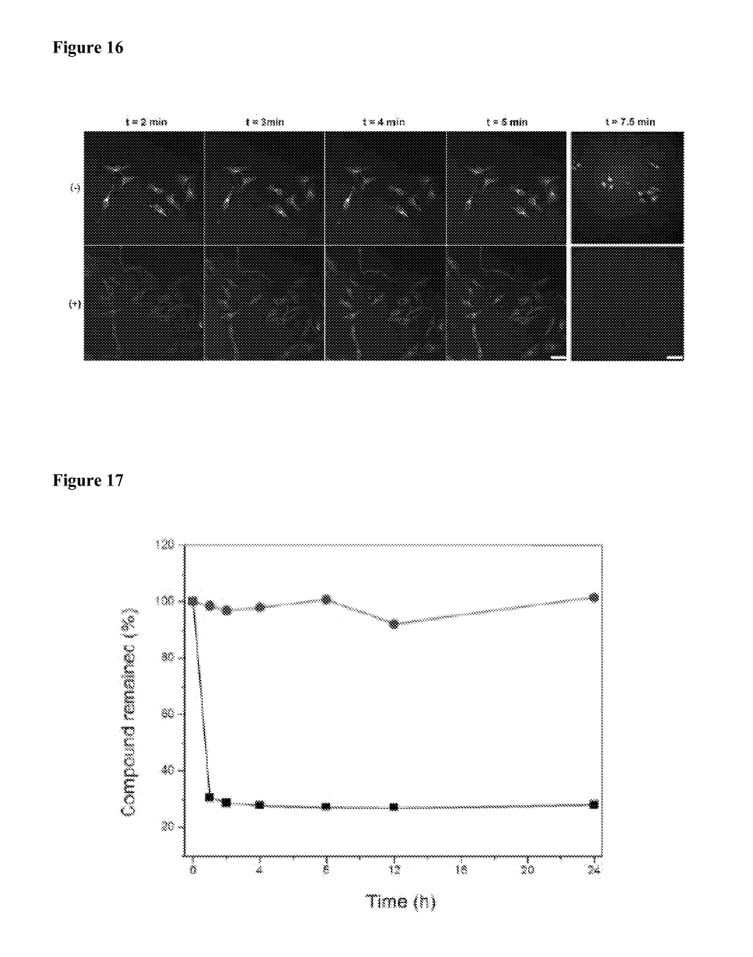

After treating HeLa cells with 500 μM of hydrogelator precursor 14b for two minutes, we observe strong fluorescence emerging from the region near the nuclei of cells (FIG. 9B, C), suggesting that the self-assembly of 15b results in formation of the nanofibers of 15b around the endoplasmic reticulum (ER). There is little fluorescence outside the cells, suggesting the lack of dephosphorylation and/or self-assembly of 15b. To confirm that the dephosphorylation of 14b and self-assembly of 15b take place in ER, we use 25 μM of CinnGEL 2Me to inhibit protein tyrosine phosphatase-1B (PTP1B), a highly efficient phosphatase located at the outer membrane of ER, when the HeLa cells are incubated with 14b (500 μM). As shown in FIG. 9D, the addition of the inhibitor of PTP significantly decreases and delays the fluorescence inside cells, confirming that the dephosphorylation of 14b and the self-assembly of 15b occur at ER. As shown by the time sequence fluorescent images of the HeLa cells incubated with 14b in the absence of the PTP1B inhibitor (FIG. 16), most of the cells exhibit strong fluorescence after treated with 14b for only 2 minutes. Even being incubated with the presence of PTP1B inhibitor, the cells still show partial fluorescence after 5 minutes of the incubation. Apparently, the fluorescence of the nanofibers in the HeLa cells treated by the D-peptide precursor (14b) emerges much faster than that of L-peptide precursor (14a) (which takes about 15 min in the presence of CinnGEL 2Me). This result agrees with that the resulted D-peptide hydrogelator (15b) is more resistant to proteolytic degradation than the L-peptide hydrogelator (15a) does.

The Application of D-Enantiomer Hydrogelator (11b) for Potential Intratumoral Chemotherapy.

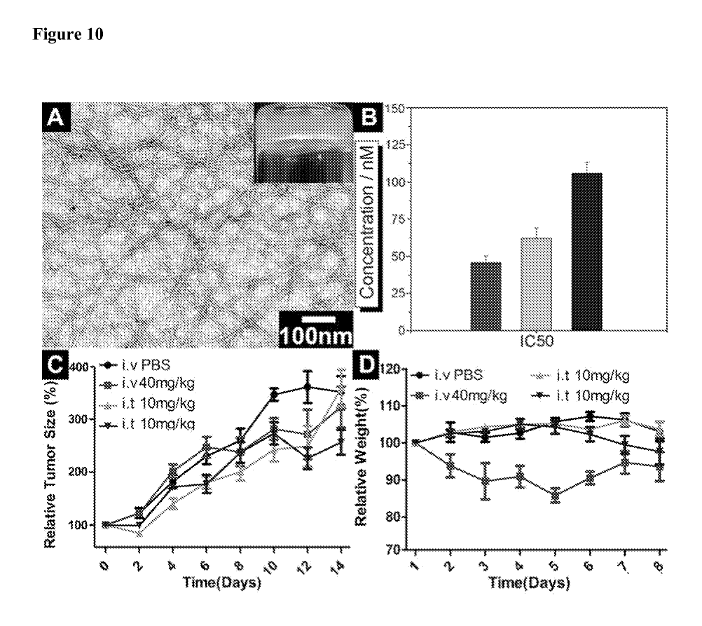

Typically, after dissolving 9.0 mg of 19b in 0.5 mL of water at pH 7.4 by sonication, we add ALP (1.0 U/mL) into the solution of 19b to obtain hydrogelator 20b, which forms a stable and semitransparent hydrogel (FIG. 10A). This result differs slightly from the behavior of precursor 19a that undergoes enzymatic hydrogelation at the concentration of 1.0 wt %, suggesting that precursor 19b (having a concentration up to 1.8 wt % for enzymatic hydrogelation) and hydrogelator 20b exhibit relatively good solubility. This subtle increase of the solubility should increase the amount of Taxol in the hydrogel. The TEM image of the hydrogel 20b shows the uniform nanofibers with the average width of 9±2 nm. To determine the efficacies of Taxol after conjugating it into the hydrogelator, we use MTT assays to examine the viability of HeLa cells incubated with Taxol (16), 19b, and 20b for 72 hours at 37° C. FIG. 10B shows the IC₅₀ values of 16, 19b, and 20b, which are 45.8 nM, 61.9 nM, and 105.9 nM, respectively. This result suggests that the conjugation of Taxol to the D-peptide essentially preserve the anti-tumor activity of Taxol, thus encouraging us to carry out in vivo test of 20b on a mouse model.

As expected, both L- and D-peptide based hydrogels of 20a and 20b exhibit similar anti-tumor activities up to 12 days of intratumoral injection of the hydrogels. After inoculating female Balb/c mice with 2×10⁵ of 4T1-luciferase cells in the mammary fat pad, we allow tumors grow until their sizes reach about 500 mm³, and randomly divide them into different treatment groups: (1) intravenous injections of PBS vehicle control; (2) intravenous injection of 4×10 mg/kg Taxol® every other day from day 0 for indicated times; (3) a single intratumoral injection of 10 mg/kg Taxol containing hydrogels in 40 μL volume. With the treatments of 16 (Taxol; paclitaxel), 20a, 20b, or PBS buffer (control) for 14 days, we monitor the relative tumor sizes (calculated by the formula: tumor volume=length×width×(Length+Width)/2) and relative weights of mice every two days. Due to the toxicity of clinical Taxol (formulated with Cremophor EL), the single injection of 40 mg/kg of Taxol® may cause the death of mouse immediately. Therefore, we have to divide 40 mg/kg of 16 into four injections with each injection of 10 mg/kg. As shown in FIG. 10C, the intravenous injections of 40 mg/kg of 16 every other day from day 0 results in the relative tumor sizes to be smaller than those of the control group after day 8. In contrast, the intratumoral injections of the hydrogel 20a or 20b at only one dose of 10 mg/kg in the mice at day 0, which may sustain for one month, reduce the relative tumor sizes on the mice more significantly than those of the controls after day 2. At day 14, although the relative tumor sizes in the groups injected with 16 and the hydrogel of 20a are similar with the PBS buffer control group, the relative tumor size in the group injected with the hydrogel of 20b is statistically smaller than the control. This result suggests that the hydrogel of 20b exhibits higher anti-tumor efficacy than 20a or 16 does. FIG. 10D shows the relative weights of mice during these 14 days treatment, suggesting that the intratumoral injection of hydrogels of 20a and 20b, only once, certainly limit the side effect of Taxol to the mice. These results support that the local injection of the hydrogels appears to achieve long term drug release with higher efficacy and better biocompatibility than the intravenous injection of Taxol. This promising result warrants further investigation of the D-peptidic hydrogels of Taxol on animal models.

### Exemplary D-Amino Acid-Containing Hydrogelators of the Invention

In certain embodiments, the invention relates to a hydrogelator of Formula I

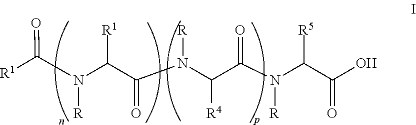

or a pharmaceutically acceptable salt thereof,
wherein, independently for each occurrence,

R is H or alkyl;

R¹ is aralkyl, heteroaralkyl, hydroxyaralkyl, or phosphorylated aralkyl;

R⁴ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or substituted aminoalkyl;

R⁵ is hydroxyaralkyl or phosphorylated aralkyl;

n is 1, 2, 3, or 4; and

p is 1, 2, 3, or 4,

provided that each amino acid residue of the hydrogelator is in the D-configuration.

In certain embodiments, the invention relates to a hydrogelator of Formula II

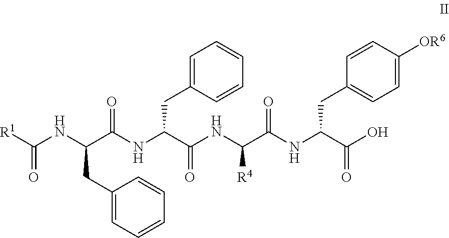

or a pharmaceutically acceptable salt thereof,
wherein, independently for each occurrence,

R¹ is aralkyl, heteroaralkyl, hydroxyaralkyl, or phosphorylated aralkyl;

R⁴ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or substituted aminoalkyl; and

R⁶ is H or P(O)(OH)₂.

In certain embodiments, the invention relates to any one of the aforementioned hydrogelators, wherein R is H.

In certain embodiments, the invention relates to any one of the aforementioned hydrogelators, wherein R¹ is aralkyl or heteroaralkyl. In certain embodiments, the invention relates to any one of the aforementioned hydrogelators, wherein R¹ is aralkyl. In certain embodiments, the invention relates to any one of the aforementioned hydrogelators, wherein R¹ is benzyl. In certain embodiments, the invention relates to any one of the aforementioned hydrogelators, wherein R¹ is naphthyl.

In certain embodiments, the invention relates to any one of the aforementioned hydrogelators, wherein R⁵ is aralkyl, hydroxyaralkyl, or phosphorylated aralkyl. In certain embodiments, the invention relates to any one of the aforementioned hydrogelators, wherein R⁵ is hydroxyaralkyl. In certain embodiments, the invention relates to any one of the aforementioned hydrogelators, wherein R⁵ is hydroxybenzyl. In certain embodiments, the invention relates to any one of the aforementioned hydrogelators, wherein R⁵ is phosphorylated aralkyl. In certain embodiments, the invention relates to any one of the aforementioned hydrogelators, wherein R⁵ is phosphorylated benzyl.

In certain embodiments, the invention relates to any one of the aforementioned hydrogelators, wherein n is 1, 2, or 3. In certain embodiments, the invention relates to any one of the aforementioned hydrogelators, wherein n is 2.

In certain embodiments, the invention relates to any one of the aforementioned hydrogelators, wherein p is 1, 2, or 3. In certain embodiments, the invention relates to any one of the aforementioned hydrogelators, wherein p is 2.

In certain embodiments, the invention relates to any one of the aforementioned hydrogelators, wherein R⁴ is aminoalkyl. In certain embodiments, the invention relates to any one of the aforementioned hydrogelators, wherein R⁴ is aminobutyl. In certain embodiments, the invention relates to any one of the aforementioned hydrogelators, wherein R⁴ is substituted aminoalkyl. In certain embodiments, the invention relates to any one of the aforementioned hydrogelators, wherein R⁴ is substituted aminobutyl. In certain embodiments, the invention relates to any one of the aforementioned hydrogelators, wherein R⁴ is A″-linker-NR-alkyl. In certain embodiments, the invention relates to any one of the aforementioned hydrogelators, wherein R⁴ is A′-NR-alkyl. In certain embodiments, the invention relates to any one of the aforementioned hydrogelators, wherein R⁴ is A″-linker-NR-butyl. In certain embodiments, the invention relates to any one of the aforementioned hydrogelators, wherein R⁴ is A′-NR-butyl. In certain embodiments, the invention relates to any one of the aforementioned hydrogelators, wherein R⁴ is A″-linker-NH-alkyl. In certain embodiments, the invention relates to any one of the aforementioned hydrogelators, wherein R⁴ is A′-NH-alkyl. In certain embodiments, the invention relates to any one of the aforementioned hydrogelators, wherein R⁴ is A″-linker-NH-butyl. In certain embodiments, the invention relates to any one of the aforementioned hydrogelators, wherein R⁴ is A′-NH-butyl.

In certain embodiments, the invention relates to any one of the aforementioned hydrogelators, wherein the hydrogelator is selected from the group consisting of:

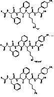

or a pharmaceutically acceptable salt thereof.

In certain embodiments, the invention relates to any one of the aforementioned hydrogelators, wherein R⁶ is H. In certain embodiments, the invention relates to any one of the aforementioned hydrogelators, wherein R⁶ is P(O)(OH)₂ or a salt thereof.

In certain embodiments, the invention relates to any one of the aforementioned hydrogelators, wherein A′ is a radical of a first active agent covalently bonded to —NH— via a carbonyl moiety (i.e., —C(O)—); and the first active agent comprises a —C(O)OR or —C(O)NR₂ moiety.

In certain embodiments, the invention relates to any one of the aforementioned hydrogelators, wherein A′ is a radical of a second active agent covalently bonded to —NH-via a carbon of an aryl, aralkyl, heteroaryl, or heteroaralkyl moiety; and the second active agent comprises an aryl halide, aralkyl halide, heteroaryl halide, or heteroaralkyl halide moiety.

In certain embodiments, the invention relates to any one of the aforementioned hydrogelators, wherein A′ is a radical of a third active agent covalently bonded to —NH— via an oxygen of an alcohol moiety or a nitrogen of an amine moiety; and the third active agent comprises an —NR₂ or —OH moiety.

In certain embodiments, the invention relates to any one of the aforementioned hydrogelators, wherein A′ is a radical of a fifth active agent covalently bonded to —NH—; and A′-NR₂ is the fifth active agent.

In certain embodiments, the invention relates to any one of the aforementioned hydrogelators, wherein the first active agent or the second active agent is an anticancer agent. In certain embodiments, the invention relates to any one of the aforementioned hydrogelators, wherein the first active agent or the second active agent is a fluorophore.

In certain embodiments, the present invention relates to any one of the aforementioned hydrogelators, wherein A′ is

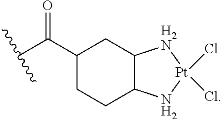

In certain embodiments, the present invention relates to any one of the aforementioned hydrogelators, wherein A′ is

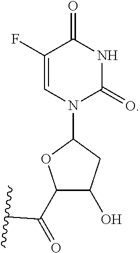 [SMILES: *C(=O)C1OC(n2cc(F)c(=O)[nH]c2=O)CC1O]

In certain embodiments, the present invention relates to any one of the aforementioned hydrogelators, wherein A′ is

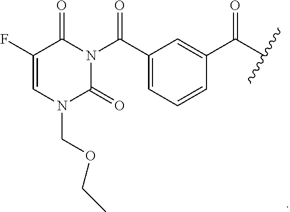 [SMILES: *C(=O)c1cccc(C(=O)n2c(=O)c(F)cn(COCC)c2=O)c1]

In certain embodiments, the present invention relates to any one of the aforementioned hydrogelators, wherein the first active agent is doxorubicin, daunorubicin, vinblastine, or vincristine.

In certain embodiments, the present invention relates to any one of the aforementioned hydrogelators, wherein the second active agent is 7-chloro-4-nitro-2,1,3-benzoxadiazole.

In certain embodiments, the present invention relates to any one of the aforementioned hydrogelators, wherein the third active agent is doxorubicin, daunorubicin, vinblastine, or vincristine.

In certain embodiments, the present invention relates to any one of the aforementioned hydrogelators, wherein the fifth active agent is doxorubicin or daunorubicin.

In certain embodiments, the invention relates to any one of the aforementioned hydrogelators, wherein A″ is a radical of a fourth active agent covalently bonded to linker via an oxygen of an alcohol moiety or a nitrogen of an amine moiety; and the fourth active agent comprises an —NR₂ or —OH moiety.

In certain embodiments, the present invention relates to any one of the aforementioned compounds, wherein A″ is

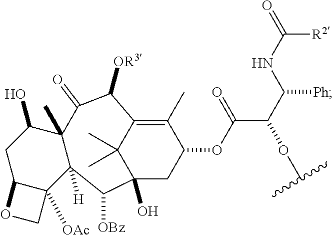

### R^{2′} is -Ph or -OtBu; and R^{3′} is —H or —C(═O)CH₃.

In certain embodiments, the present invention relates to any one of the aforementioned hydrogelators, wherein A″ is

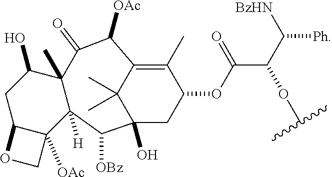

In certain embodiments, the present invention relates to any one of the aforementioned hydrogelators, wherein A″ is

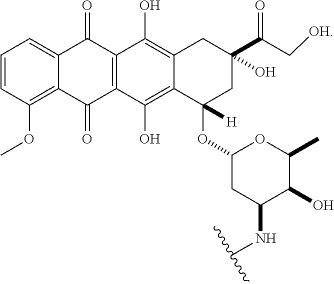

In certain embodiments, the present invention relates to any one of the aforementioned hydrogelators, wherein the fourth active agent is doxorubicin.

In certain embodiments, the present invention relates to any one of the aforementioned hydrogelators, wherein A″ is

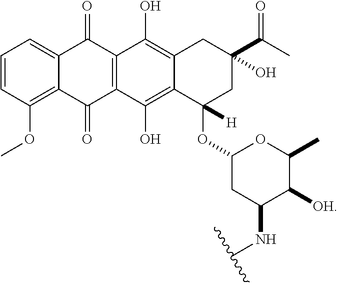

In certain embodiments, the present invention relates to any one of the aforementioned hydrogelators, wherein the fourth active agent is daunorubicin.

In certain embodiments, the present invention relates to any one of the aforementioned hydrogelators, wherein the fourth active agent is vinblastine or vincristine.

In certain embodiments, the invention relates to any one of the aforementioned hydrogelators, wherein the linker is —C(O)—(C₁-C₈-alkylene)-C(O)—. In certain embodiments, the invention relates to any one of the aforementioned hydrogelators, wherein the linker is —C(O)—(C₁-C₃-alkylene)-C(O)—. In certain embodiments, the invention relates to any one of the aforementioned hydrogelators, wherein the linker is —C(O)—CR₂CR₂—C(O)—. In certain embodiments, the invention relates to any one of the aforementioned hydrogelators, wherein the linker is —C(O)—CH₂CH₂—C(O)—.

In certain embodiments, the invention relates to a hydrogelator selected from the group consisting of:

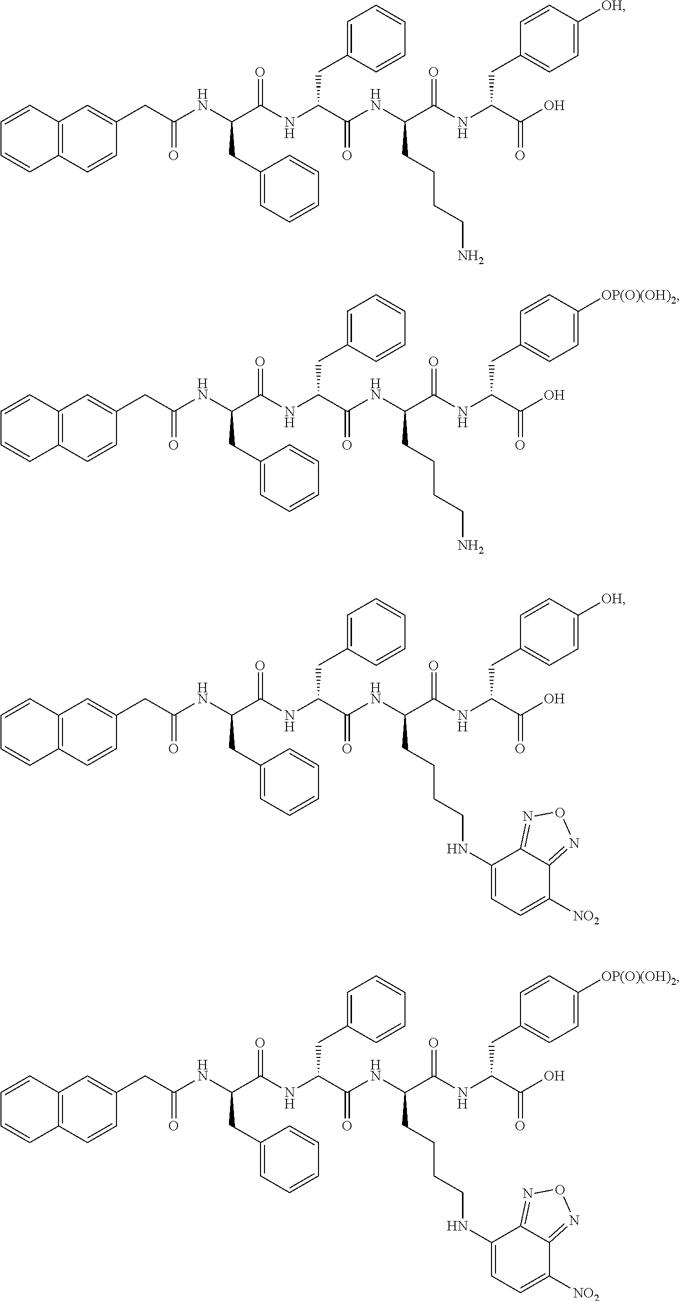

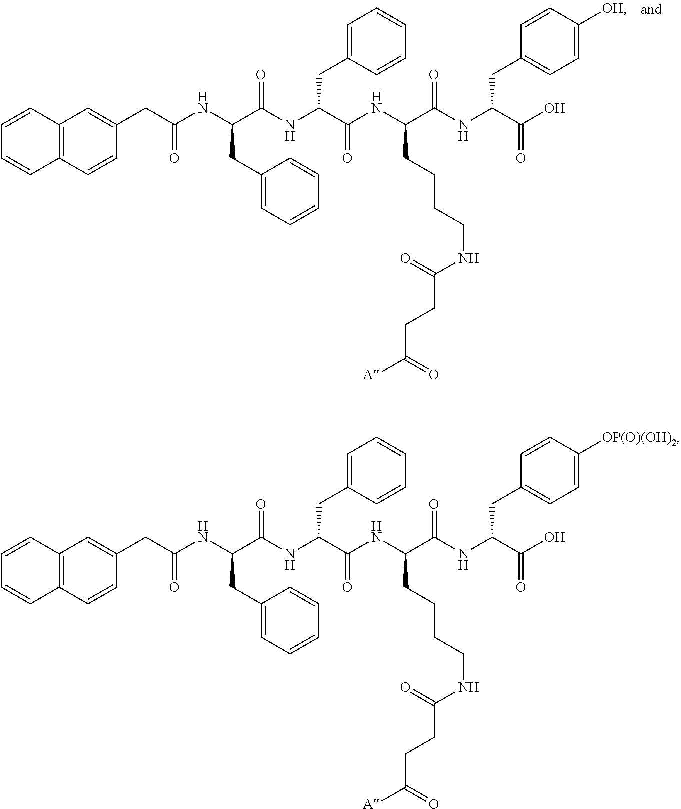

wherein A″ is

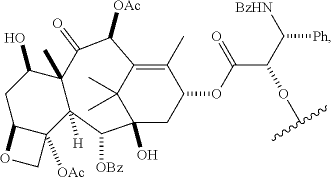

or a pharmaceutically acceptable salt thereof.

### Exemplary Supramolecular Structures of the Invention

In certain embodiments, the invention relates to a supramolecular structure comprising, consisting essentially of, or consisting of a plurality of any one of the aforementioned hydrogelators. In certain embodiments, the invention relates to a supramolecular structure comprising, consisting essentially of, or consisting of a plurality of a hydrogelator of Formula I or Formula II.

In certain embodiments, the invention relates to any one of the aforementioned supramolecular structures, wherein the supramolecular structure is in the form of nanofibers. In certain embodiments, the average diameter of the nanofibers is about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 30 nm, about 35 nm, about 40 nm, about 45 nm, about 50 nm, about 55 nm, about 60 nm, about 65 nm, about 70 nm, about 75 nm, or about 80 nm. In certain diameters, the nanofibers are substantially straight. In certain embodiments, the nanofibers are bent. In certain embodiments, the nanofibers form networks. In certain embodiments, the nanofibers are bent. In certain embodiments, the nanofibers form bundles. In certain embodiments, the nanofibers are about 100 nm, about 120 nm, about 140 nm, about 160 nm, about 180 nm, about 200 nm, about 220 nm, about 240 nm, about 260 nm, about 280 nm, or about 300 nm in length. In certain embodiments, the nanofibers are greater than about 100 nm, about 120 nm, about 140 nm, about 160 nm, about 180 nm, about 200 nm, about 220 nm, about 240 nm, about 260 nm, about 280 nm, or about 300 nm in length. In certain embodiments, the average diameter is calculated as the average width of a nanofiber, as depicted via TEM.

### Exemplary Hydrogels of the Invention

In certain embodiments, the invention relates to a hydrogel, comprising, consisting essentially of, or consisting of a plurality of any one of the aforementioned hydrogelators; and water. In certain embodiments, the invention relates to a hydrogel, comprising, consisting essentially of, or consisting of a plurality of hydrogelators of Formula I or hydrogelators of Formula II; and water.

In certain embodiments, the invention relates to a hydrogel, comprising, consisting essentially of, or consisting of a plurality of any one of the aforementioned supramolecular structures; and water.

In certain embodiments, the invention relates to any one of the aforementioned hydrogels, wherein the hydrogel is formed from a solution of the hydrogelators in water. In certain embodiments, the hydrogelator is present in an amount of about 0.2% to about 4% by weight. In certain embodiment, the hydrogelator is present in an amount of about 0.2%, about 0.4%, about 0.6%, about 0.8%, about 1.0%, about 1.5%, about 2.0%, about 2.5%, about 3.0%, about 3.5%, or about 4.0% by weight.

In certain embodiments, the invention relates to any one of the aforementioned hydrogels, wherein the hydrogel is formed from a solution of the hydrogelators in water. In certain embodiments, the temperature of the solution is about 20° C., about 25° C., or about 30° C.

In certain embodiments, the invention relates to any one of the aforementioned hydrogels, wherein the hydrogel is formed by decreasing the pH of the solution of hydrogelators in water. In certain embodiments, the pH at which the supramolecular structure is formed is about 8.0, about 7.5, about 7.0, about 6.5, about 6.0, about 5.5, about 5.0, about 4.5, or about 4.0.

In certain embodiments, the invention relates to any one of the aforementioned hydrogels, wherein the hydrogel is formed by the addition of an enzyme to the solution of hydrogelators in water. In certain embodiments, the enzyme is a phosphatase. In certain embodiments, the enzyme is alkaline phosphatase.

In certain embodiments, the invention relates to any one of the aforementioned hydrogels, wherein the hydrogel has a critical strain value of about 0.2% to about 25.0%. In certain embodiments, the invention relates to any one of the aforementioned hydrogels, wherein the hydrogel has a critical strain value of about 0.2%, about 0.3%, about 0.4%, about 0.5%, about 0.6%, about 0.7%, about 0.8%, about 0.9%, about 1.0%, about 1.2%, about 1.4%, about 1.6%, about 1.8%, about 2.0%, about 2.2%, about 2.4%, about 2.6%, about 2.8%, about 3.0%, about 3.2%, about 3.4%, about 3.6%, about 3.8%, about 4.0%, about 4.2%, about 4.4%, about 4.6%, about 4.8%, about 5.0%, about 5.2%, about 5.4%, about 5.6%, about 5.8%, about 6.0%, about 6.2%, about 6.4%, about 6.6%, about 6.8%, about 7.0%, about 7.2%, about 7.4%, about 7.6%, about 7.8%, about 8.0%, about 8.2%, about 8.4%, about 8.6%, about 8.8%, about 9.0%, about 9.2%, about 9.4%, about 9.6%, about 9.8%, about 10%, about 11%, about 12%, about 13%, about 14%, about 15%, about 16%, about 17%, about 18%, about 19%, about 20%, about 21%, about 22%, about 23%, about 24%, or about 25%.

In certain embodiments, the invention relates to any one of the aforementioned hydrogels, wherein the hydrogel has a storage modulus of about 75 Pa to about 70 KPa. In certain embodiments, the invention relates to any one of the aforementioned hydrogels, wherein the hydrogel has a storage modulus of about 75 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1.0 KPa, about 1.5 KPa, about 2.0 KPa, about 2.5 KPa, about 3.0 KPa, about 3.5 KPa, about 4.0 KPa, about 4.5 KPa, about 5.0 KPa, about 5.5 KPa, about 6.0 KPa, about 6.5 KPa, about 7.0 KPa, about 7.5 KPa, about 8.0 KPa, about 8.5 KPa, about 9.0 KPa, about 9.5 KPa, about 10.0 KPa, about 15 KPa, about 20 KPa, about 25 KPa, about 30 KPa, about 35 KPa, about 40 KPa, about 45 KPa, about 50 KPa, about 55 KPa, about 60 KPa, about 65 KPa, or about 70 KPa.

In certain embodiments, the invention relates to any one of the aforementioned hydrogels, wherein the hydrogel is substantially biocompatible. In certain embodiments, the invention relates to any one of the aforementioned hydrogels, wherein the hydrogel is substantially biostable.

### Exemplary Methods of the Invention

In certain embodiments, the invention relates to a method of treating cancer, tumors, malignancies, neoplasms, or other dysproliferative diseases, comprising

administering to a subject in need thereof a therapeutically effective amount of any one of the aforementioned hydrogelators, any one of the aforementioned supramolecular structures, or any one of the aforementioned hydrogels, wherein the hydrogelator comprises a radical of an active agent; and the active agent is an anticancer agent.

In certain embodiments, the invention relates to any one of the aforementioned methods, wherein the cancer, tumor, malignancy, neoplasm, or other dysproliferative disease is selected from the group consisting of leukemias, lymphomas, myeloproliferative diseases, and solid tumors.

In certain embodiments, the invention relates to any one of the aforementioned methods, wherein the cancer, tumor, malignancy, neoplasm, or other dysproliferative disease is selected from the group consisting of myeloid leukemia, lymphocytic leukemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In certain embodiments, the invention relates to a method of in vivo imaging, comprising

administering to a subject in need thereof a diagnostically effective amount of any one of the aforementioned hydrogelators, any one of the aforementioned supramolecular structures, or any one of the aforementioned hydrogels, wherein the hydrogelator comprises a radical of an active agent; and the active agent is a fluorophore.

### NSAID Hydrogelator Design, Synthesis, and Discussion

Since the formation of supramolecular hydrogels relies on small molecules that self-assemble in water via non-covalent interactions (i.e., hydrogelators), and since the sustained drug-release depends on the biostability of the hydrogels, principles for designing a hydrogelator comprising a NSAIDs include, but are not limited to, (i) enabling the self-assembly of NSAIDs without compromising the activity of the NSAIDs; (ii) resisting the premature degradation due to proteolytic hydrolysis. In certain embodiments, the invention relates to a hydrogelator comprising a prescription NSAID, such as naproxen (Npx). In certain embodiments, the hydrogelator is the condensation product between an oligopeptides and a NSAID. In certain embodiments, the invention relates to a hydrogelator comprising diphenylalanine (Phe-Phe). In certain embodiments, the Phe-Phe motif enables functional molecules to self-assemble in water. In certain embodiments, the hydrogelator comprises D-Phe-D-Phe and Npx. In certain embodiments, the use of D-amino acids for the conjugates confers proteolytic resistance to the hydrogelators. In certain embodiments, the use of D-amino acids for the conjugates enhances the selectivity of the hydrogelators for inhibiting COX-2. In certain embodiments, the invention relates to a hydrogelator comprising a NSAID, wherein the hydrogelator exhibits improved selectivity over the NSAID alone. In certain embodiments, the invention relates to a hydrogelator comprising a NSAID, wherein the hydrogelator is biostable, target specific, and/or potent.

In certain embodiments, the invention relates to a compound, comprising, consisting essentially of, or consisting of a fragment of a NSAID; and an oligopeptide.

In certain embodiments, the invention relates to a hydrogel formed by an enzymatic reaction upon a compound of the invention. In certain embodiments, the invention relates to a hydrogel formed from a compound of the invention upon a change in pH.

In certain embodiments, the invention relates to a soft, biocompatible material, comprising, consisting essentially of, or consisting of a compound of the invention.

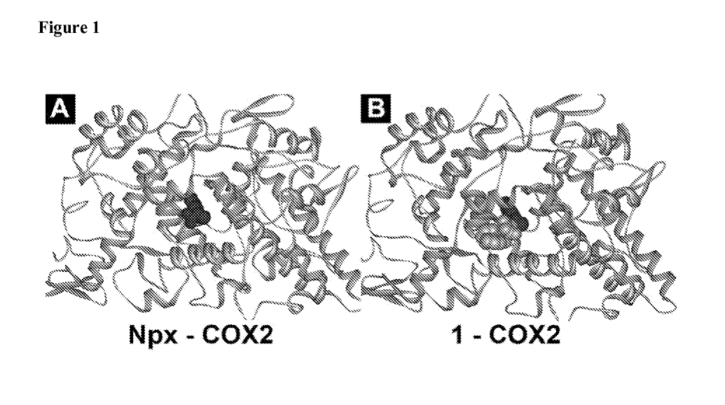

In certain embodiments, the hydrogelators of the invention were designed based on the crystal structure of COX-2, which suggests that the conjugation of amino acids to Npx hardly disrupts the binding of Npx to COX-2. FIG. 1 shows an example of the design. According to the binding of Npx (center) with COX-2 enzyme (gray) (FIG. 1A), the carboxylate end of Npx is available for modification after Npx binds to COX-2 due to the large open space in the structure of COX-2. FIG. 1B shows the predicted binding model of hydrogelator Npx-D-Phe-D-Phe (1, Npx-ff, spheres represent D-Phe-D-Phe) and COX-2: the connection of a rather bulky D-Phe-D-Phe dipeptide to Npx still allows the Npx to bind to the active site of COX-2. In certain embodiments, the oligopeptides further comprises D-tyrosine phosphate. In certain embodiments, the Npx fragment is covalently bonded to the side chain of a D-amino acid for evaluating the correlation between the structure and the activity of the hydrogelators of NSAIDs. FIG. 4 shows the molecular structures of exemplary derivatives of Npx. The connection of D-Phe-D-Phe, D-Phe-D-Phe-D-Tyr, D-Phe-D-Phe-D-Lys or D-Phe-D-Phe-D-Lys-D-Tyr to Npx results in molecules Npx-ff (1), Npx-ffy (2), Npx-ffk (3), or Npx-ffky (4), respectively, that contains Npx at the backbone of the small peptide. The conjugation of Npx to the side chain of D-Phe-D-Phe-D-Lys or D-Phe-D-Phe-D-Lys-D-Tyr via the ε-amino group of the D-Lys residue produces molecules ffk(Npx) (5) and ffk(Npx)y (6). The addition of a phosphate group on the tyrosine residue of 2 and 4 affords the precursors (2P and 4P) that would convert to molecules 2 and 4 followed by the dephosphorylation catalyzed by phosphatases.

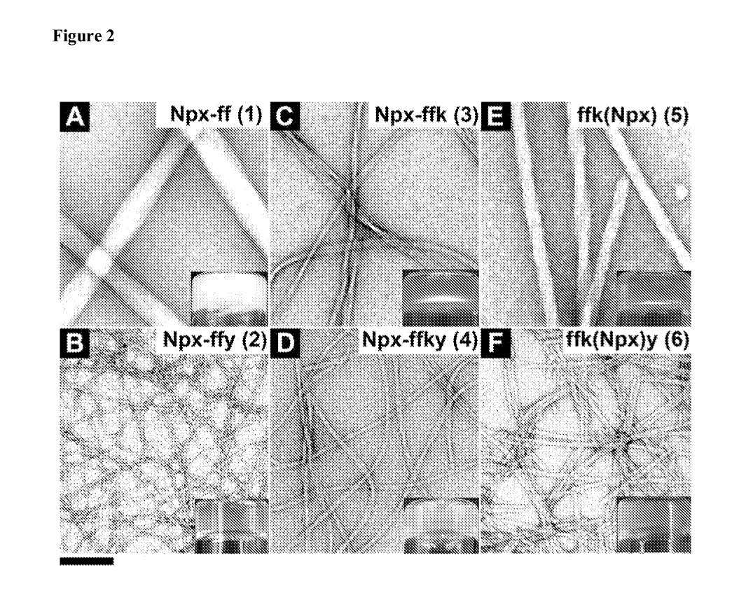

In certain embodiments, we synthesized the molecules in FIG. 4 according to the synthetic procedures that combine solid phase synthesis and N-hydroxysuccinimide (NHS) assisted coupling reaction. See Yan, C. Q.; et al. Langmuir 2012, 28, 6076-6087. After the synthesis of the designed hydrogelators, the gelation test indicated that all the hydrogelators in FIG. 4 are able to form stable hydrogels at the concentration of 0.8 wt % (FIG. 2), but the hydrogels exhibit a slightly different appearance. For example, the aid of sonication and heating afforded the aqueous solution of 1 at pH 9.0, which turns into an opaque hydrogel upon the adjustment of the pH to 4.0 at room temperature. Unlike the case of 1, the addition of 1 U/mL of alkaline phosphatase into the solution of 2P resulted in a transparent hydrogel of 2 at pH 7.6. By changing the pH and temperature, we obtained the hydrogels of 3, 5, and 6, respectively. By adding 1 U/mL of alkaline phosphatase into the solution of 4P, we obtained the hydrogel of 4.

Transmission electron microscopy (TEM) was used to examine the Npx-containing hydrogels for evaluating the characteristics of the molecular assemblies. As shown in FIGS. 2A and 2B, hydrogelator 1 self-assembles to afford large and rigid nanofibers with average width of 54±7 nm, while hydrogelator 2 gives long, thin, and flexible nanofibers with average width of 7±2 nm (FIG. 2B). FIG. 2C shows the nanofibers with helical structure formed by a hydrogel of 3, of which average width is 16±3 nm. The enzymatically formed hydrogel of 4 forms long and flexible nanofibers with average width of 10±2 nm (FIG. 2D). A hydrogel of 5 exhibits helical, rigid, and long nanofibers with average widths of 26±3 nm (FIG. 2E), meanwhile, hydrogelator 6 self-assembles to give rigid but short nanofibers with average widths of 7±2 nm, which tend to form bundles (FIG. 2F). As shown in the bottom row of the images in FIG. 2, the hydrogels containing D-Tyr (i.e., hydrogels of 2, 4, and 6) exhibit smaller diameter nanofibers that entangle to form a network with higher density than their corresponding hydrogels (i.e., hydrogels of 1, 3, and 5) without D-tyrosine. The incorporation of D-Lys in hydrogelator 3 also makes it able to form more flexible and narrower nanofibers than the nanofibers of hydrogelator 1. Hydrogel 4 contains nanofibers that have similar morphologies to those in hydrogel 2. The hydrogels of 5 and 6, which have Npx connected at the side chain, contain nanofibers that are rigid and straight, which differ from those flexible and long nanofibers in the hydrogels of 3 and 4. The differences in the morphologies of these hydrogels indicate that the position of Npx and the presence of tyrosine at the C-terminus likely play a role in their self-assembly in water.

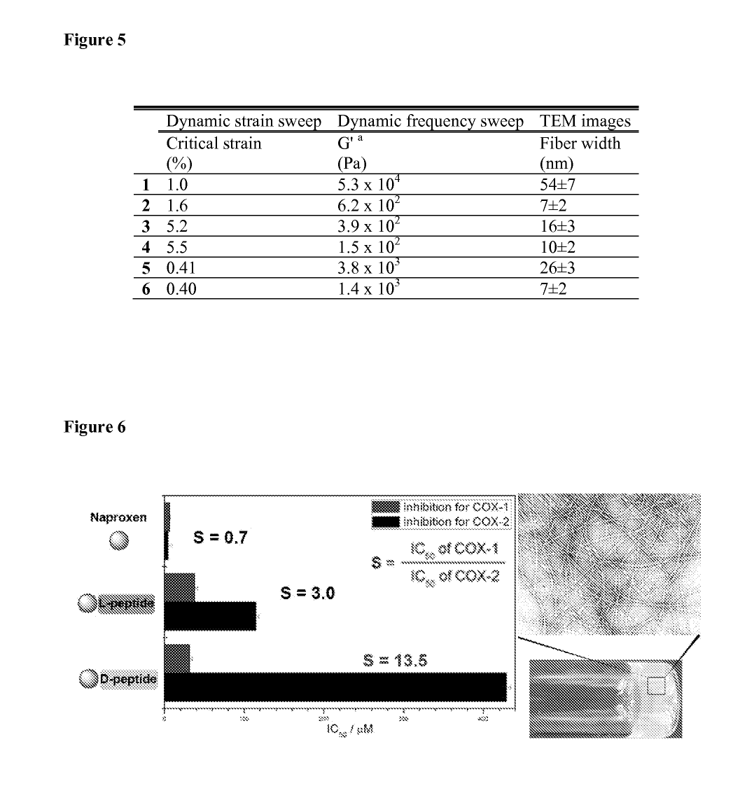

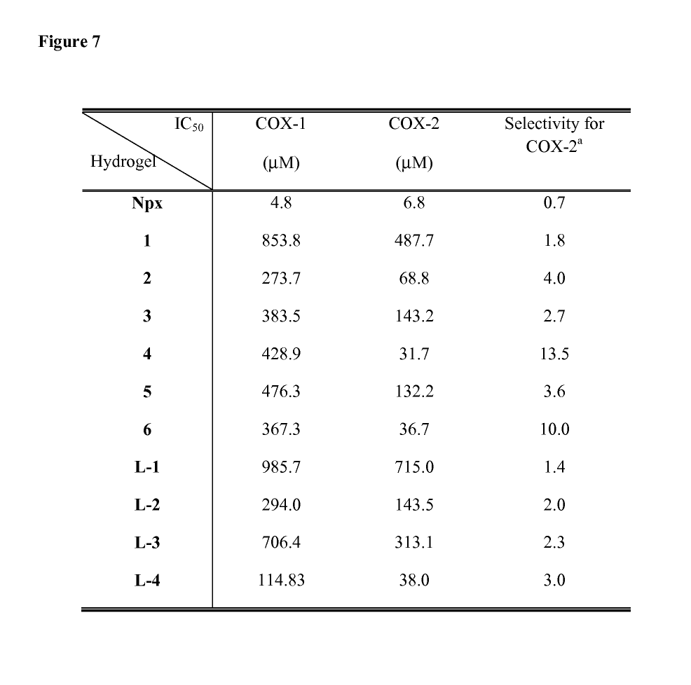

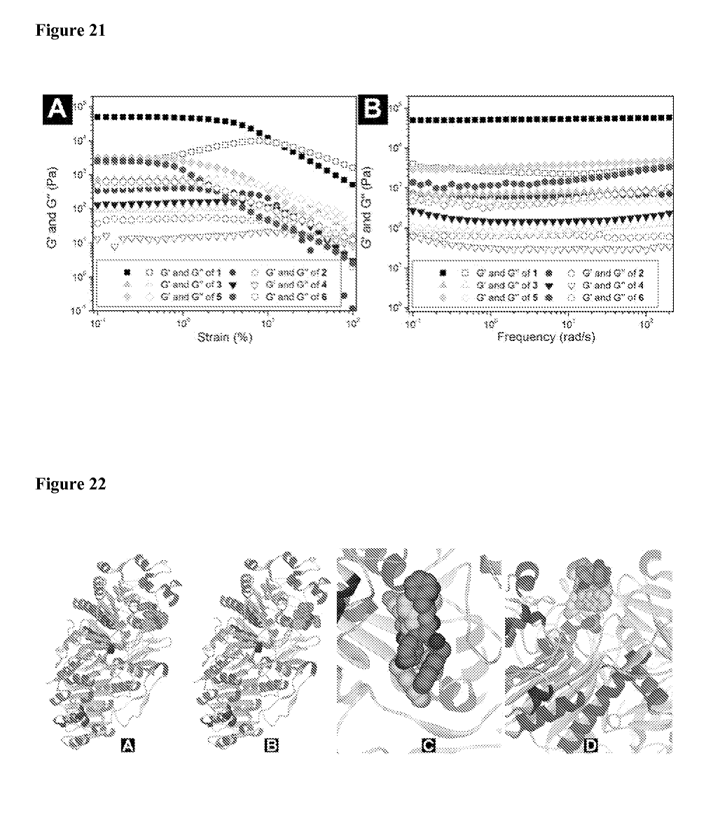

Oscillatory rheology was used to examine the viscoelastic properties of the hydrogels. The Npx-containing hydrogels studied all exhibited viscoelastic properties of a solid-like material because the storage moduli (G′) were significantly higher than the loss moduli (G″). In addition, the storage moduli of the hydrogels were frequency independent (FIG. 21). As summarized in FIG. 5, the critical strains of hydrogels 1, 2, 3, 4, 5, and 6 are 1.0%, 1.6%, 5.2%, 5.5%, 0.41% and 0.40%, respectively; their values of G′ (at the frequency of 6.28 rad/s) in dynamic frequency sweep rad/s are 5.3×10⁴, 6.2×10², 3.9×10², 1.5×10², 3.8×10³, and 1.4×10³ Pa, respectively. The relatively large critical strains of 3 and 4 suggest that the ε-amino group from the lysine residue makes the networks of the hydrogels to be resilient. The low critical strains of hydrogels 1, 5, and 6 apparently agree with the rigidity of the nanofibers in those hydrogels, which also confers relatively high storage moduli (G′). These results provide insights on the correlation between molecular structures of the hydrogelators and the viscoelasticity of the supramolecular hydrogels.

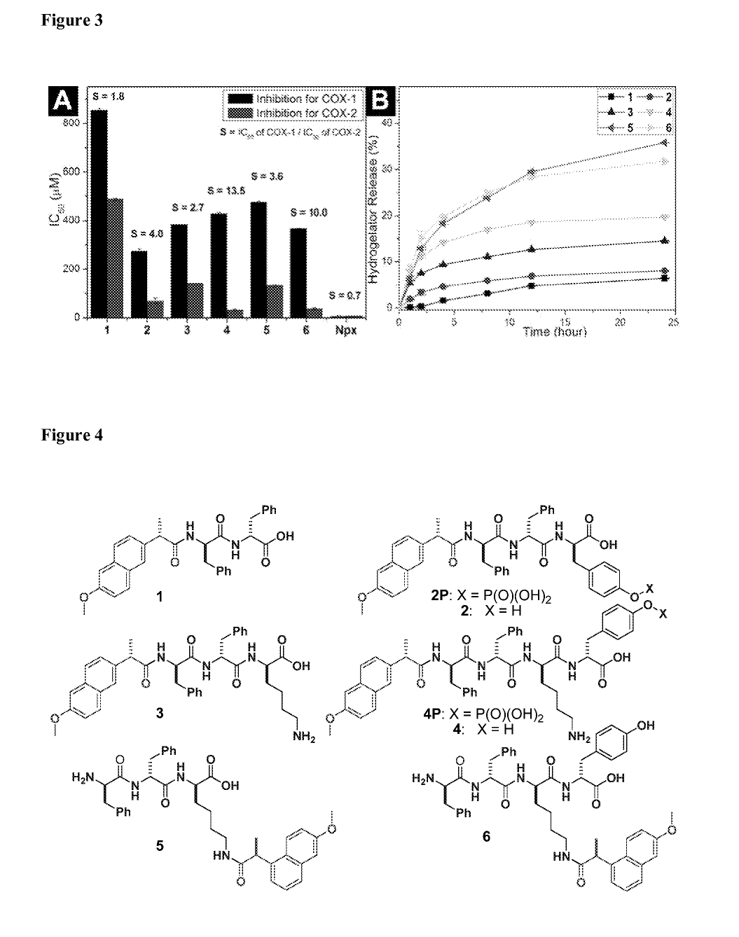

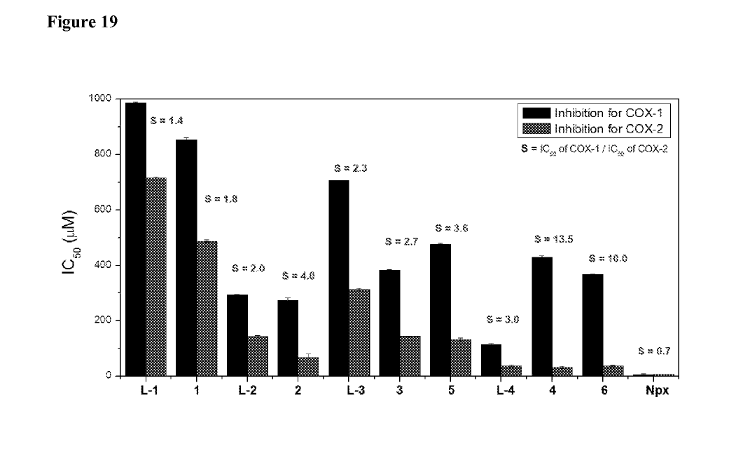

In vitro inhibition assays were performed for both COX-1 and COX-2 enzymes to evaluate the efficacies of the NSAID containing hydrogelators. As shown in FIG. 3A, the IC₅₀ values of COX-1 enzyme of hydrogelators 1, 2, 3, 4, 5, and 6 are 853.8, 273.7, 383.5, 428.9, 476.3, and 367.3 μM, respectively. All these value are almost two orders of magnitude higher than the reported IC₅₀ values of naproxen (0.6-4.8 μM) in the literature. The attachment of the small D-peptides to naproxen may reduce its binding to COX-1, which may reduce the associated adverse gastrointestinal and renal effects. For COX-2, an inducible enzyme at the site of inflammation, the IC₅₀ values of hydrogelators 1, 2, 3, 4, 5, and 6 are 487.7, 68.8, 143.2, 31.7, 132.2, and 36.7 μM, respectively. Since the reported IC₅₀ of naproxen to COX-2 is 2.0-28.4 μM, hydrogelators 4 and 6, afford reasonable IC₅₀ values for the inhibition of COX-2. Thus, hydrogelators 4 and 6 exhibit excellent selectivity, S=13.5 and S=10.0, respectively, towards COX-2. These results not only validate 4 and 6 as potential candidates for topical NSAID gels, but also suggest that the presence of D-tyrosine on the D-peptides is beneficial for the activity and selectivity regardless the position of Npx on either the side chain or the main chain of the D-peptide. In the control compound, the use of L-amino acids (L-Phe, L-Lys, and L-Tyr) to replace the D-amino acid residues in hydrogelators 1, 2, 3, and 4 results in hydrogelators that exhibit higher IC₅₀ values with poorer selectivity towards COX-2 (FIG. 19). For example, L-4 (Npx-FFKY) exhibits IC₅₀ values of 38.0 and 114.8 μM for COX-2 and COX-1, respectively, which affords the selectivity for COX-2 inhibition to be about 3. These results indicate the advantages of using D-peptide for generating the hydrogelators containing Npx to achieve high selectivity.

After studying their drug efficacies, the sustained release of Npx-containing hydrogelators from 0.8 wt % of hydrogels was studied. We incubated 100 μL of hydrogels at 37° C. for 24 hours with 100 μL of PBS buffer solution (pH 7.4), which is refreshed and monitored at 1 h, 2 h, 4 h, 8 h, 12 h, and 24 h. FIG. 3B shows the release profile of these hydrogelators. After 24 hours, hydrogel 1 slowly and steadily released 6.5% of the hydrogelator. With increased solubility contributed from hydrophilic amino acid residues (i.e., Tyr and Lys), hydrogels 2, 3, and 4 release 8.0%, 14.5%, and 19.8%, respectively, of the hydrogelator. Hydrogels 5 and 6 release 35.8% and 31.7% of hydrogelators after 24 h. These results suggest that these Npx containing hydrogels may serve as topical gels for sustained drug delivery.

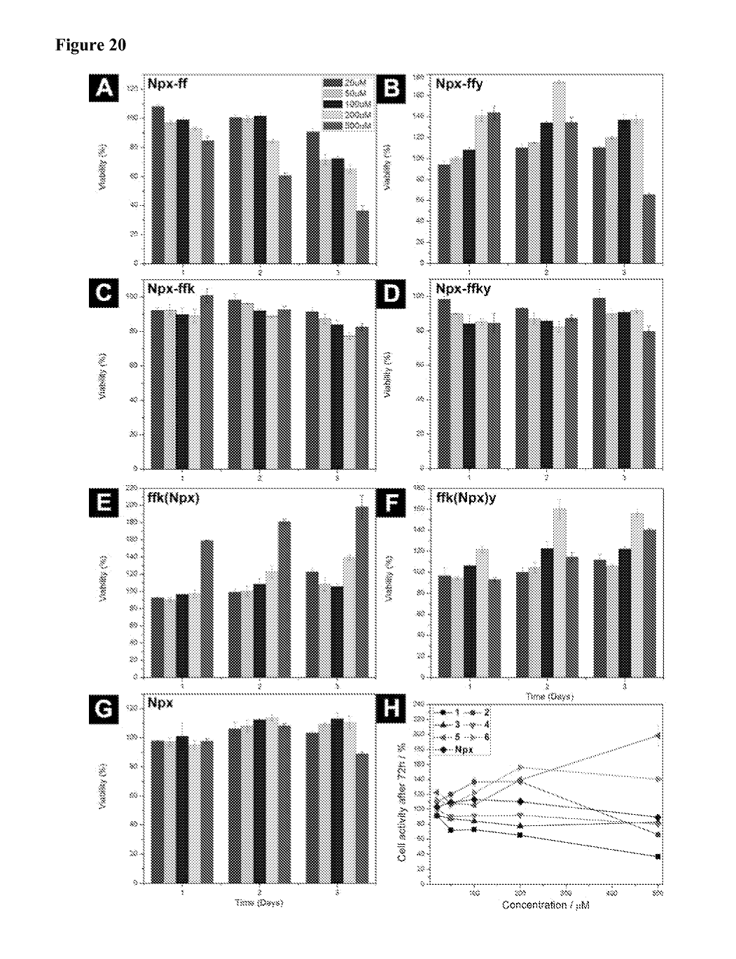

The biocompatibility of the Npx containing hydrogelators was examined by incubating them with HeLa cells for 72 hours at 37° C. As shown in FIG. 20, the hydrogelators have IC₅₀ values higher than 500 μM, except 1 which exhibits IC₅₀ value of 357 μM. The high IC₅₀ values of the hydrogels may indicate that they are cell compatible. Although the absorption of formazan in the MTT assay indicates the promotion of the growth of the cells when the cells were incubated with hydrogelators 2, 5, or 6, we found no promotion of the cell proliferation based on the change of the numbers of the HeLa cells.

### Exemplary NSAID Hydrogelators of the Invention

In certain embodiments, the invention relates to a hydrogelator of Formula III

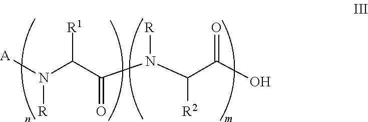

or a pharmaceutically acceptable salt thereof,
wherein, independently for each occurrence,

A is selected from the group consisting of

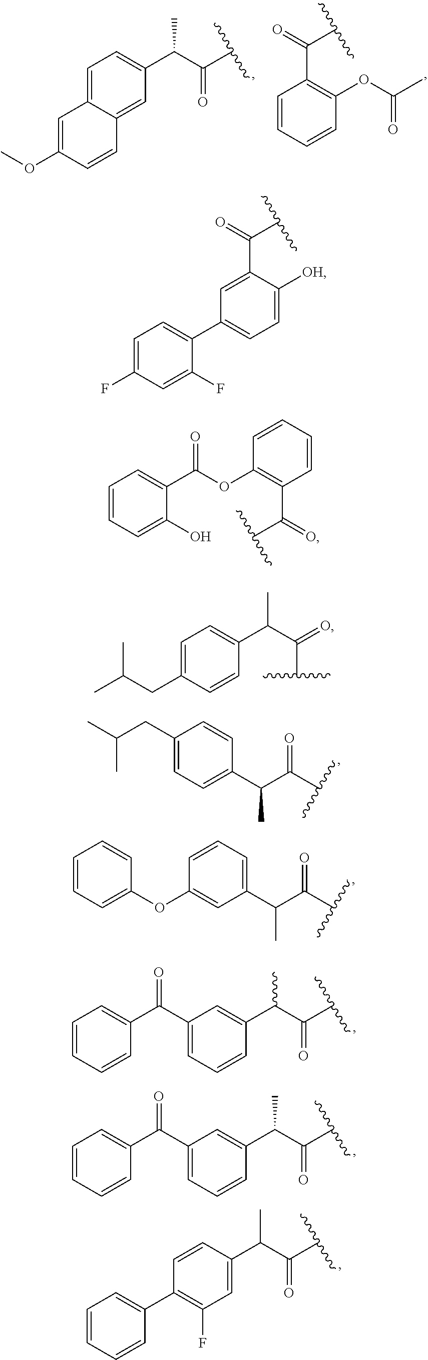

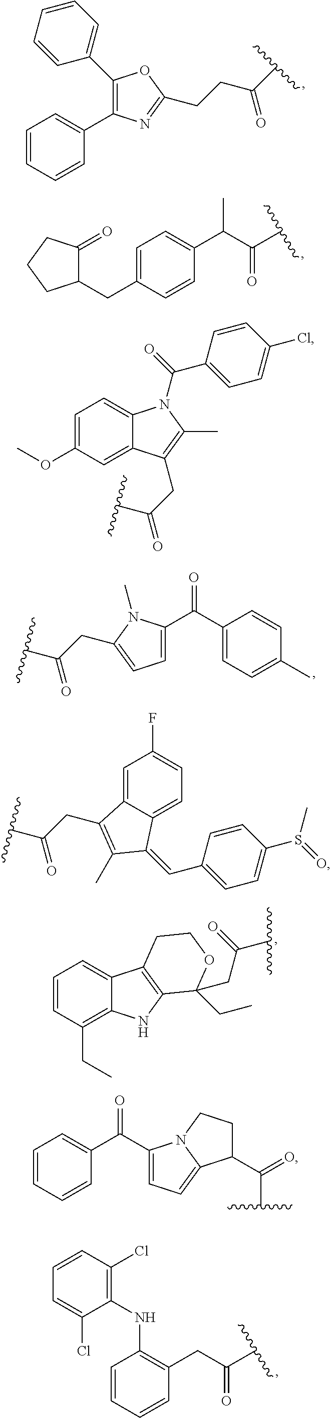

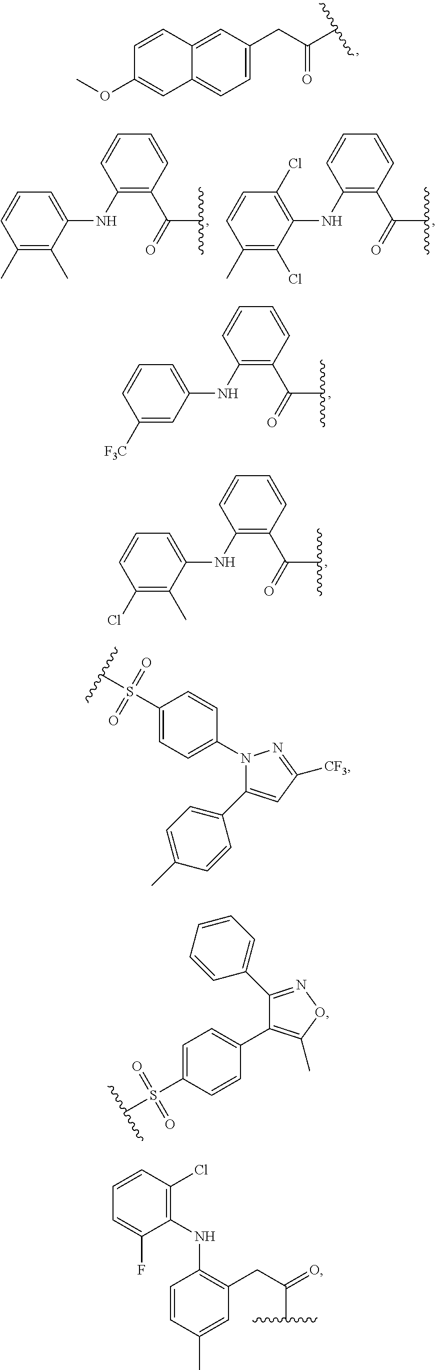

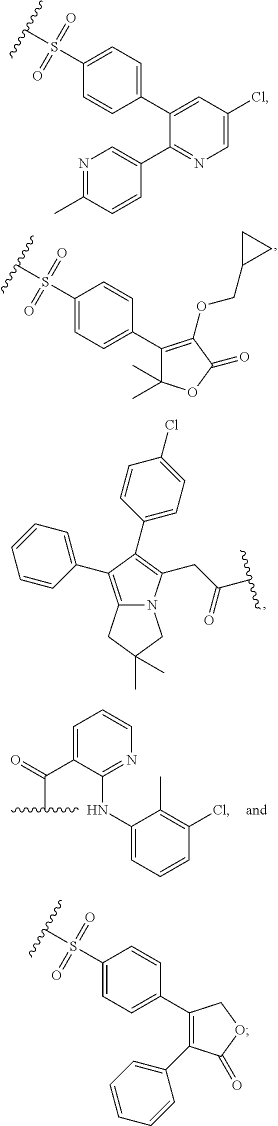

R is H or alkyl;

R¹ is aralkyl, heteroaralkyl, hydroxyaralkyl, or phosphorylated aralkyl;

R² is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, or HS-alkyl;

n is 1, 2, 3, or 4; and

m is 0, 1, 2, 3, or 4.

In certain embodiments, the invention relates to a hydrogelator of Formula IV

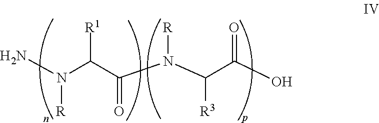

or a pharmaceutically acceptable salt thereof,
wherein, independently for each occurrence,

R is H or alkyl;

R¹ is aralkyl, heteroaralkyl, hydroxyaralkyl, or phosphorylated aralkyl;

R³ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or A-NR-alkyl, provided at least one instance of R³ is A-NR-alkyl;
n is 1, 2, 3, or 4;
p is 1, 2, 3, or 4; and

A is selected from the group consisting of

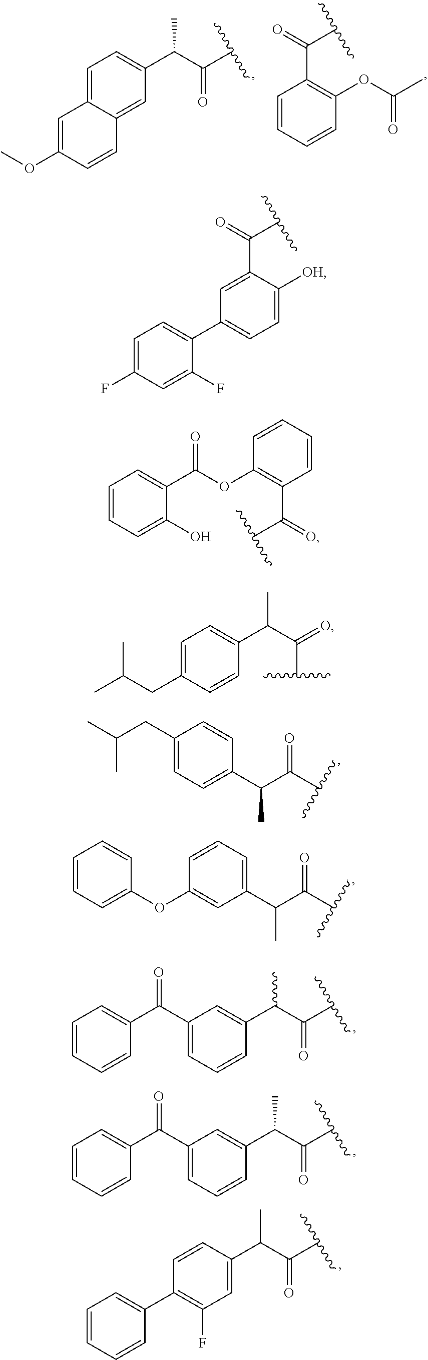

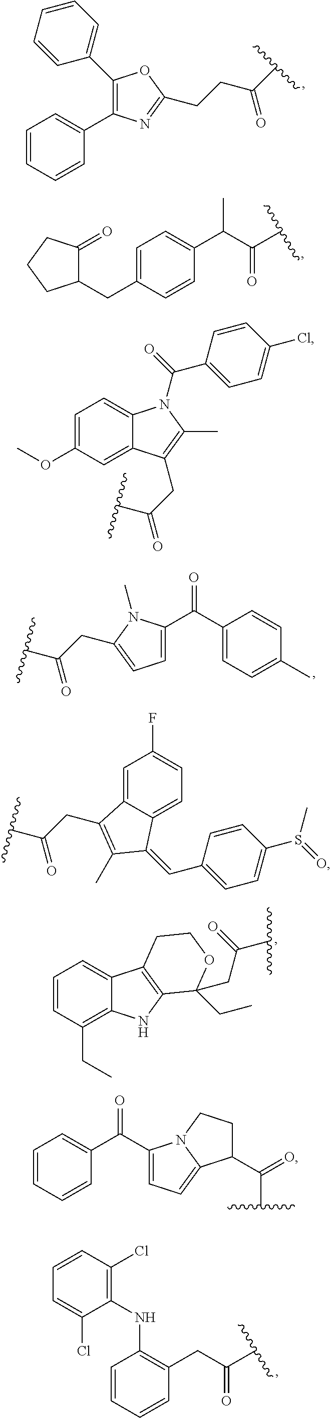

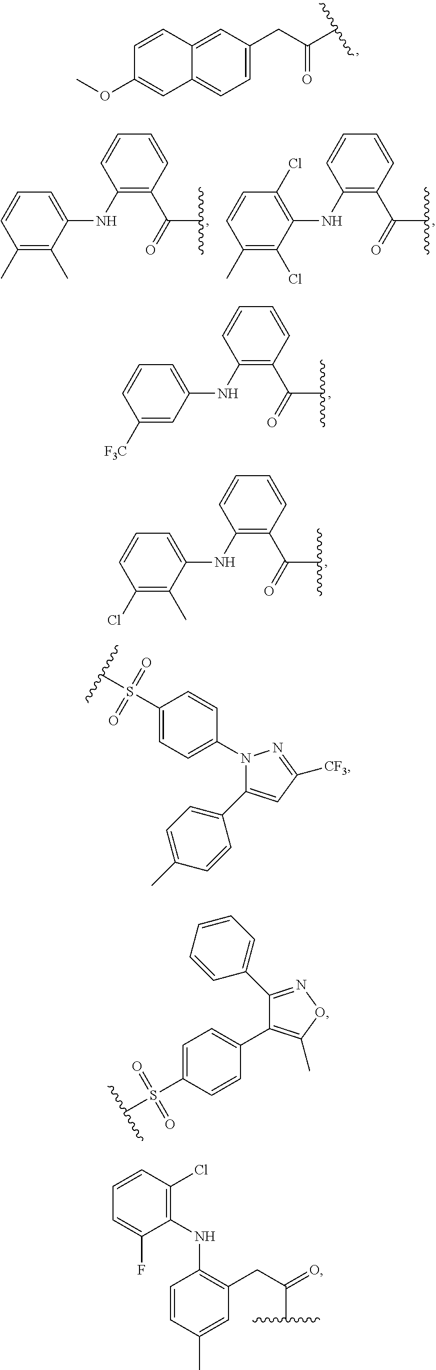

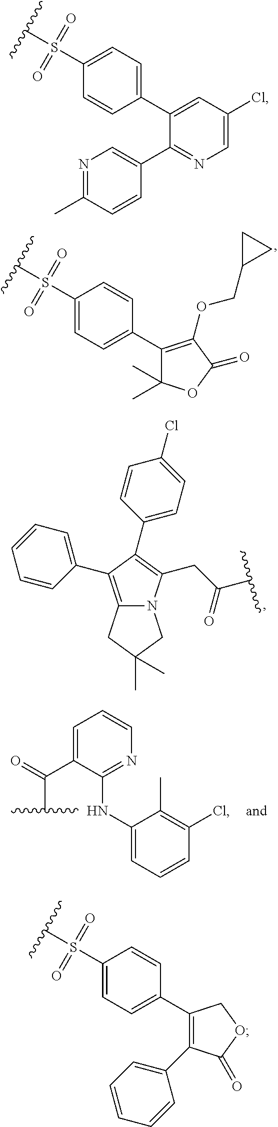

In certain embodiments, the invention relates to any one of the aforementioned hydrogelators, wherein A is selected from the group consisting of

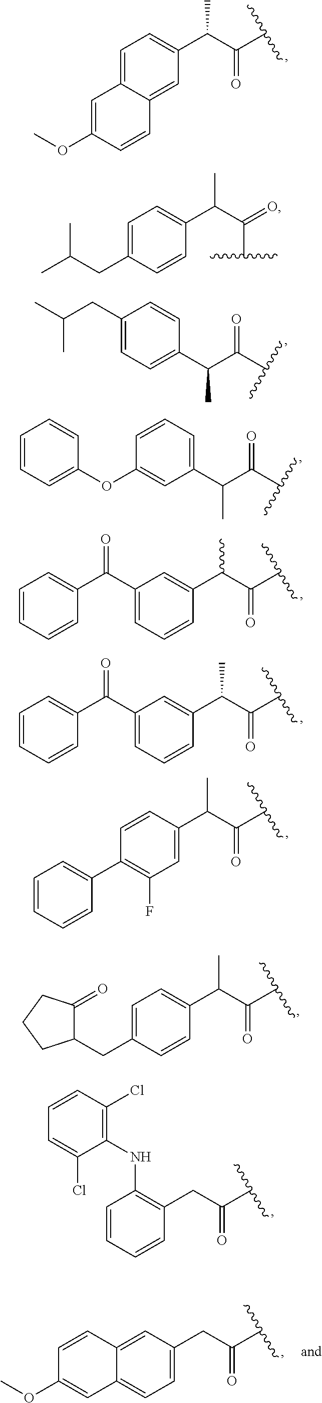

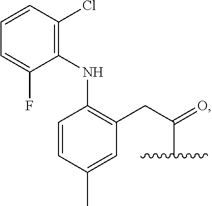 [SMILES: *C(=O)Cc1cc(C)ccc1Nc1c(F)cccc1Cl]

In certain embodiments, the invention relates to any one of the aforementioned hydrogelators, wherein A is

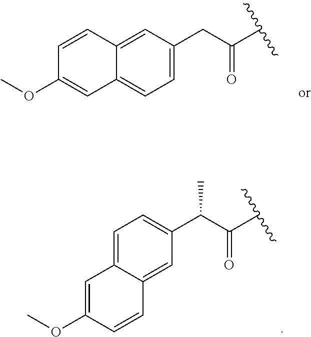 [SMILES: *C(=O)Cc1ccc2cc(OC)ccc2c1 ; *C(=O)[C@@H](C)c1ccc2cc(OC)ccc2c1]

In certain embodiments, the invention relates to any one of the aforementioned hydrogelators, wherein R is H.

In certain embodiments, the invention relates to any one of the aforementioned hydrogelators, wherein R¹ is aralkyl or heteroaralkyl. In certain embodiments, the invention relates to any one of the aforementioned hydrogelators, wherein R¹ is aralkyl. In certain embodiments, the invention relates to any one of the aforementioned hydrogelators, wherein R¹ is benzyl.

In certain embodiments, the invention relates to any one of the aforementioned hydrogelators, wherein R² is aralkyl, hydroxyaralkyl, phosphorylated aralkyl, alkyl, aminoalkyl, or hydroxyalkyl. In certain embodiments, the invention relates to any one of the aforementioned hydrogelators, wherein R² is hydroxyaralkyl. In certain embodiments, the invention relates to any one of the aforementioned hydrogelators, wherein R² is hydroxybenzyl. In certain embodiments, the invention relates to any one of the aforementioned hydrogelators, wherein R² is phosphorylated aralkyl. In certain embodiments, the invention relates to any one of the aforementioned hydrogelators, wherein R² is phosphorylated benzyl. In certain embodiments, the invention relates to any one of the aforementioned hydrogelators, wherein R² is aminoalkyl. In certain embodiments, the invention relates to any one of the aforementioned hydrogelators, wherein R² is aminobutyl.

In certain embodiments, the invention relates to any one of the aforementioned hydrogelators, wherein n is 1, 2, or 3. In certain embodiments, the invention relates to any one of the aforementioned hydrogelators, wherein n is 2.

In certain embodiments, the invention relates to any one of the aforementioned hydrogelators, wherein m is 0, 1, or 2. In certain embodiments, the invention relates to any one of the aforementioned hydrogelators, wherein m is 0. In certain embodiments, the invention relates to any one of the aforementioned hydrogelators, wherein m is 1.

In certain embodiments, the invention relates to any one of the aforementioned hydrogelators, wherein R³ is A-NR-alkyl. In certain embodiments, the invention relates to any one of the aforementioned hydrogelators, wherein R³ is A-NR-butyl. In certain embodiments, the invention relates to any one of the aforementioned hydrogelators, wherein R³ is A-NH-alkyl. In certain embodiments, the invention relates to any one of the aforementioned hydrogelators, wherein R³ is A-NH-butyl.

In certain embodiments, the invention relates to any one of the aforementioned hydrogelators, wherein p is not 1; and one instance of R³ is hydroxyaralkyl. In certain embodiments, the invention relates to any one of the aforementioned hydrogelators, wherein p is not 1; and one instance of R³ is hydroxylbenzyl.

In certain embodiments, the invention relates to any one of the aforementioned hydrogelators, wherein p is 1, 2, or 3. In certain embodiments, the invention relates to any one of the aforementioned hydrogelators, wherein p is 1. In certain embodiments, the invention relates to any one of the aforementioned hydrogelators, wherein p is 2.

In certain embodiments, the invention relates to any one of the aforementioned hydrogelators, wherein each chiral carbon of the oligopeptide is in the R-configuration.

In certain embodiments, the invention relates to any one of the aforementioned hydrogelators, wherein each amino acid residue is in the D-configuration.

In certain embodiments, the invention relates to a hydrogelator selected from the group consisting of:

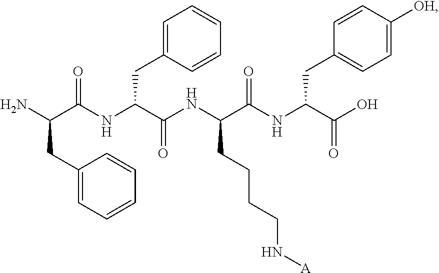

wherein A is selected from the group consisting

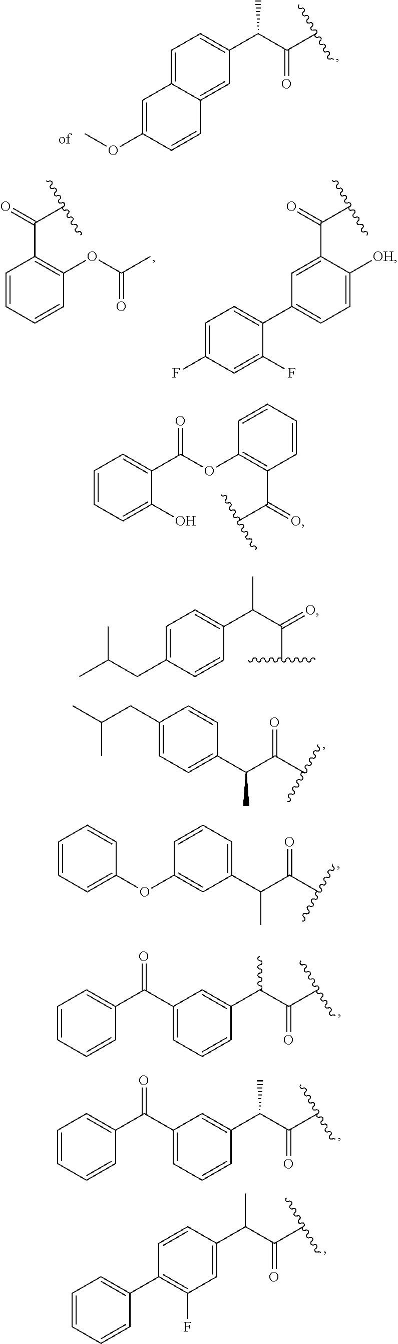

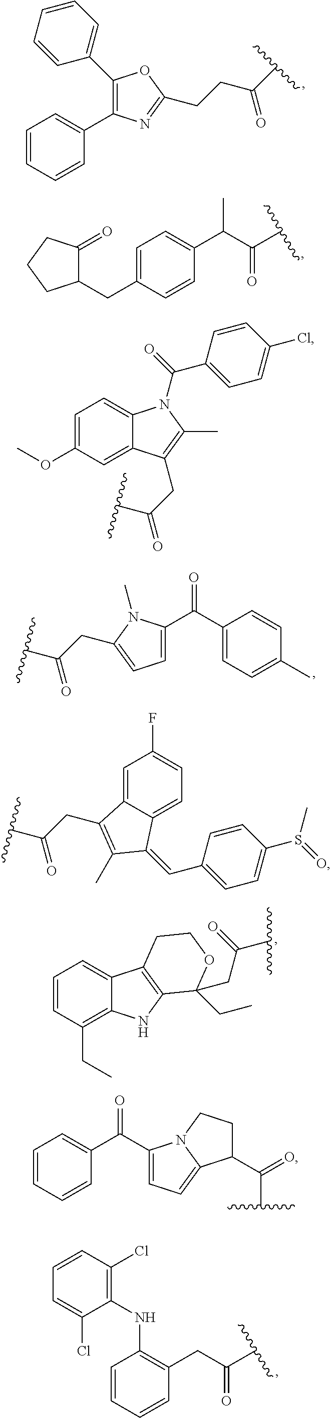

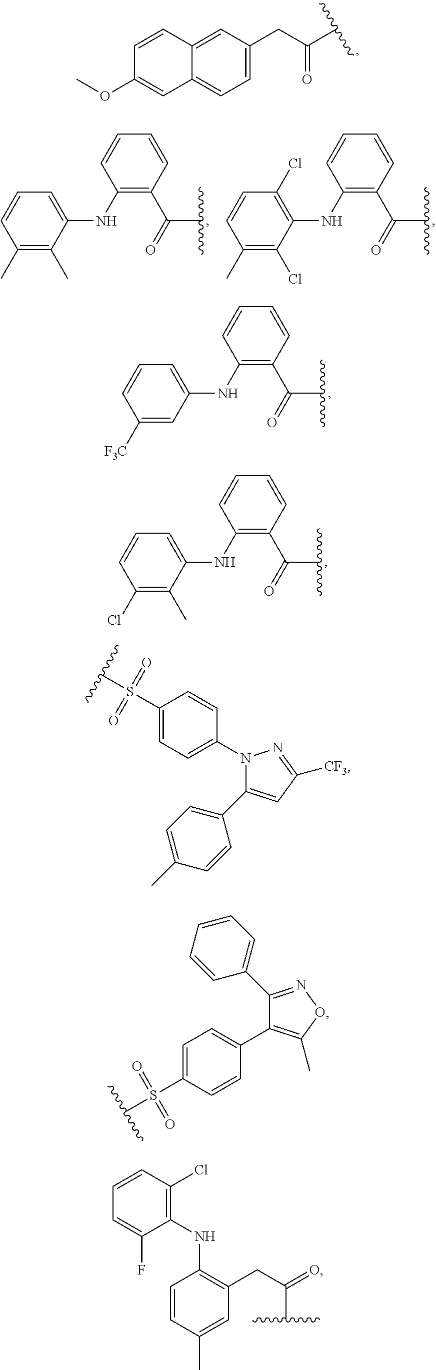

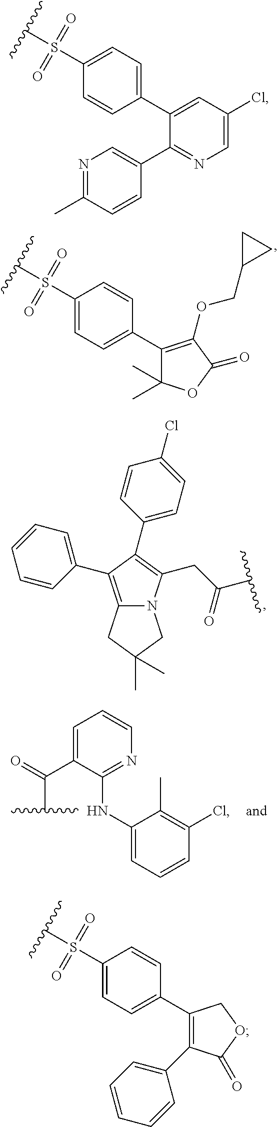

or a pharmaceutically acceptable salt thereof

In certain embodiments, the invention relates to a hydrogelator selected from the group consisting of:

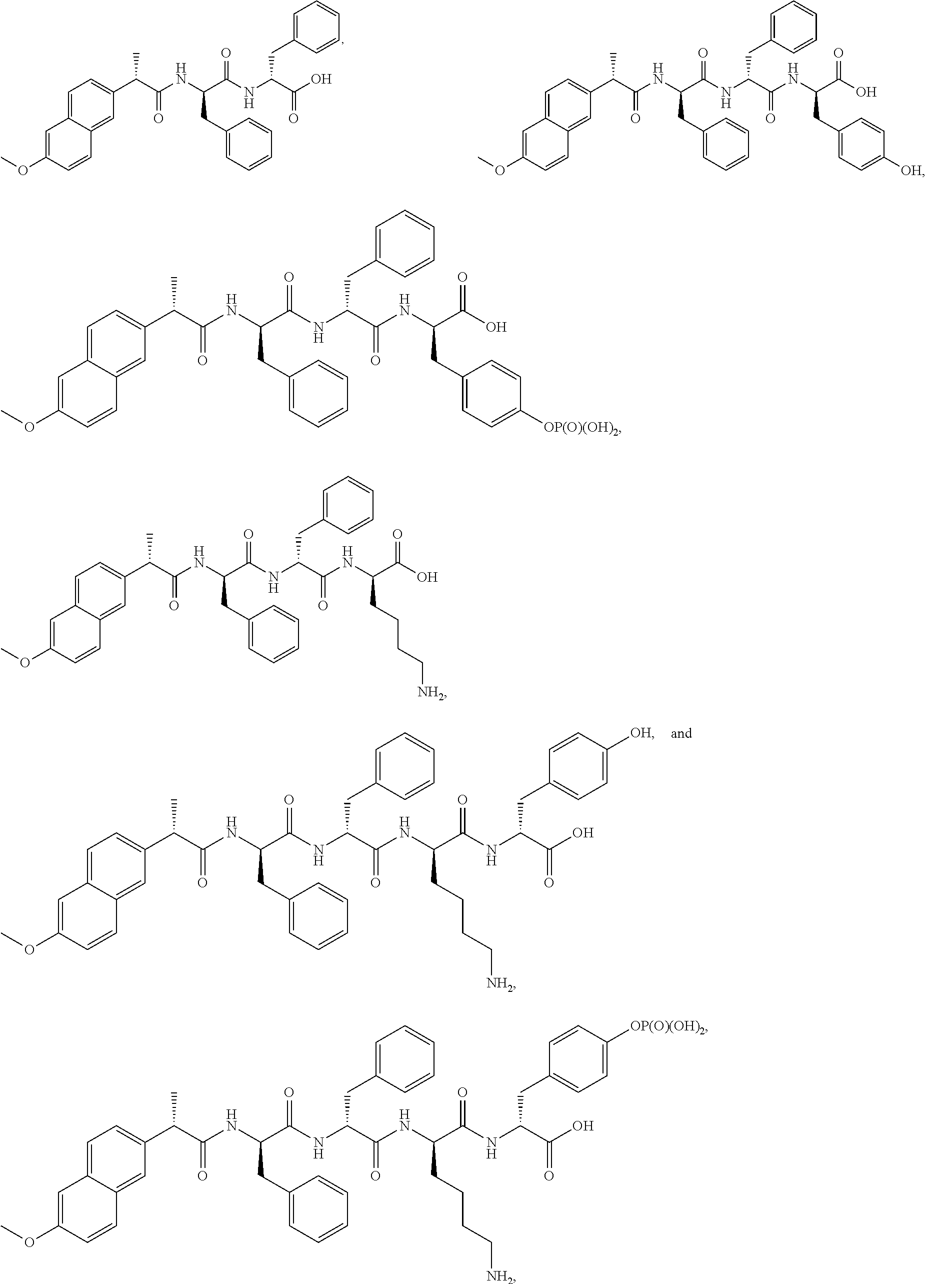

or a pharmaceutically acceptable salt thereof

In certain embodiments, the invention relates to a hydrogelator selected from the group consisting of:

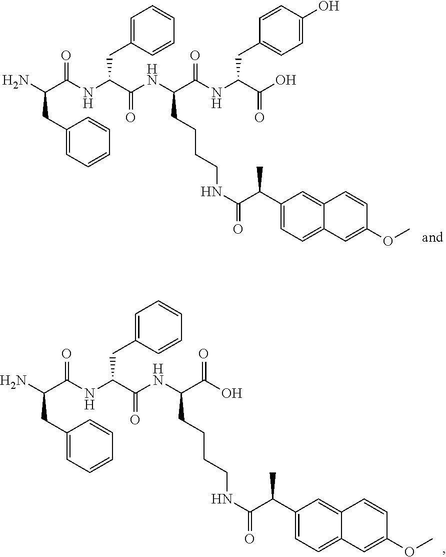 [SMILES: COc1ccc2cc([C@H](C)C(=O)NCCCC[C@@H](NC(=O)[C@@H](Cc3ccccc3)NC(=O)[C@H](N)Cc3ccccc3)C(=O)N[C@H](Cc3ccc(O)cc3)C(=O)O)ccc2c1 ; COc1ccc2cc([C@H](C)C(=O)NCCCC[C@@H](NC(=O)[C@@H](Cc3ccccc3)NC(=O)[C@H](N)Cc3ccccc3)C(=O)O)ccc2c1]

or a pharmaceutically acceptable salt thereof.

In certain embodiments, the invention relates to any one of the aforementioned hydrogelators, wherein the hydrogelator exhibits a selectivity for inhibition of COX-2 over COX-1 of at least about 2, at least about 3, at least about 4, at least about 5, or at least about 6. In certain embodiments, the invention relates to any one of the aforementioned hydrogelators, wherein the hydrogelator exhibits a selectivity for inhibition of COX-2 over COX-1 of about 2, about 3, about 4, about 5, about 6, about 7, about 8, about 9, about 10, about 11, about 12, about 13, about 14, about 15, about 16, about 17, about 18, about 19, or about 20. In certain embodiments, selectivity is calculated as the ratio of IC₅₀ of COX-1/IC₅₀ of COX-2.

### Exemplary Supramolecular Structures of the Invention

In certain embodiments, the invention relates to a supramolecular structure comprising, consisting essentially of, or consisting of a plurality of any one of the aforementioned hydrogelators. In certain embodiments, the invention relates to a supramolecular structure comprising a plurality of compounds of Formula III or a plurality of compounds of Formula IV.

In certain embodiments, the invention relates to any one of the aforementioned supramolecular structures, wherein the supramolecular structure is in the form of nanofibers. In certain embodiments, the average diameter of the nanofibers is about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 30 nm, about 35 nm, about 40 nm, about 45 nm, about 50 nm, about 55 nm, about 60 nm, about 65 nm, about 70 nm, about 75 nm, or about 80 nm. In certain diameters, the nanofibers are substantially straight. In certain embodiments, the nanofibers are bent. In certain embodiments, the nanofibers form networks. In certain embodiments, the nanofibers are bent. In certain embodiments, the nanofibers form bundles. In certain embodiments, the nanofibers are about 100 nm, about 120 nm, about 140 nm, about 160 nm, about 180 nm, about 200 nm, about 220 nm, about 240 nm, about 260 nm, about 280 nm, or about 300 nm in length. In certain embodiments, the nanofibers are greater than about 100 nm, about 120 nm, about 140 nm, about 160 nm, about 180 nm, about 200 nm, about 220 nm, about 240 nm, about 260 nm, about 280 nm, or about 300 nm in length. In certain embodiments, the average diameter is calculated as the average width of a nanofiber, as depicted via TEM.

### Exemplary Hydrogels of the Invention

In certain embodiments, the invention relates to a hydrogel, comprising, consisting essentially of, or consisting of a plurality of any one of the aforementioned hydrogelators; and water. In certain embodiments, the invention relates to a hydrogel comprising a plurality of compounds of Formula III or a plurality of compounds of Formula IV; and water.

In certain embodiments, the invention relates to a hydrogel, comprising, consisting essentially of, or consisting of a plurality of any one of the aforementioned supramolecular structures; and water.

In certain embodiments, the invention relates to any one of the aforementioned hydrogels, wherein the hydrogel is formed from a solution of the hydrogelators in water. In certain embodiments, the hydrogelator is present in an amount of about 0.2% to about 4% by weight. In certain embodiment, the hydrogelator is present in an amount of about 0.2%, about 0.4%, about 0.6%, about 0.8%, about 1.0%, about 1.5%, about 2.0%, about 2.5%, about 3.0%, about 3.5%, or about 4.0% by weight.

In certain embodiments, the invention relates to any one of the aforementioned hydrogels, wherein the hydrogel is formed from a solution of the hydrogelators in water. In certain embodiments, the temperature of the solution is about 20° C., about 25° C., or about 30° C.

In certain embodiments, the invention relates to any one of the aforementioned hydrogels, wherein the hydrogel is formed by decreasing the pH of the solution of hydrogelators in water. In certain embodiments, the pH at which the supramolecular structure is formed is about 8.0, about 7.5, about 7.0, about 6.5, about 6.0, about 5.5, about 5.0, about 4.5, or about 4.0.

In certain embodiments, the invention relates to any one of the aforementioned hydrogels, wherein the hydrogel is formed by the addition of an enzyme to the solution of hydrogelators in water. In certain embodiments, the enzyme is a phosphatase. In certain embodiments, the enzyme is alkaline phosphatase.

In certain embodiments, the invention relates to any one of the aforementioned hydrogels, wherein the hydrogel has a critical strain value of about 0.2% to about 10.0%. In certain embodiments, the invention relates to any one of the aforementioned hydrogels, wherein the hydrogel has a critical strain value of about 0.2%, about 0.3%, about 0.4%, about 0.5%, about 0.6%, about 0.7%, about 0.8%, about 0.9%, about 1.0%, about 1.2%, about 1.4%, about 1.6%, about 1.8%, about 2.0%, about 2.2%, about 2.4%, about 2.6%, about 2.8%, about 3.0%, about 3.2%, about 3.4%, about 3.6%, about 3.8%, about 4.0%, about 4.2%, about 4.4%, about 4.6%, about 4.8%, about 5.0%, about 5.2%, about 5.4%, about 5.6%, about 5.8%, about 6.0%, about 6.2%, about 6.4%, about 6.6%, about 6.8%, about 7.0%, about 7.2%, about 7.4%, about 7.6%, about 7.8%, about 8.0%, about 8.2%, about 8.4%, about 8.6%, about 8.8%, about 9.0%, about 9.2%, about 9.4%, about 9.6%, about 9.8%, or about 10%.

In certain embodiments, the invention relates to any one of the aforementioned hydrogels, wherein the hydrogel has a storage modulus of about 75 Pa to about 70 KPa. In certain embodiments, the invention relates to any one of the aforementioned hydrogels, wherein the hydrogel has a storage modulus of about 75 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1.0 KPa, about 1.5 KPa, about 2.0 KPa, about 2.5 KPa, about 3.0 KPa, about 3.5 KPa, about 4.0 KPa, about 4.5 KPa, about 5.0 KPa, about 5.5 KPa, about 6.0 KPa, about 6.5 KPa, about 7.0 KPa, about 7.5 KPa, about 8.0 KPa, about 8.5 KPa, about 9.0 KPa, about 9.5 KPa, about 10.0 KPa, about 15 KPa, about 20 KPa, about 25 KPa, about 30 KPa, about 35 KPa, about 40 KPa, about 45 KPa, about 50 KPa, about 55 KPa, about 60 KPa, about 65 KPa, or about 70 KPa.

In certain embodiments, the invention relates to any one of the aforementioned hydrogels, wherein the hydrogel is substantially biocompatible. In certain embodiments, the invention relates to any one of the aforementioned hydrogels, wherein the hydrogel is substantially biostable.

### Exemplary Methods of the Invention

In certain embodiments, the invention relates to a method of treating an inflammatory condition, comprising

administering to a subject in need thereof a therapeutically effective amount of any one of the aforementioned hydrogelators, any one of the aforementioned supramolecular structures, or any one of the aforementioned hydrogels.

In certain embodiments, the invention relates to any one of the aforementioned methods, wherein the hydrogelator is a compound of Formula III or a compound of Formula IV.

In certain embodiments, the invention relates to any one of the aforementioned methods, wherein the hydrogelator, the supramolecular structure, or the hydrogel is administered topically.

In certain embodiments, the invention relates to any one of the aforementioned methods, wherein the hydrogelator, the supramolecular structure, or the hydrogel is administered to the skin of the subject in need thereof.

In certain embodiments, the invention relates to any one of the aforementioned methods, wherein the hydrogelator, the supramolecular structure, or the hydrogel is in the form of a lotion, cream, or gel.

In certain embodiments, the invention relates to any one of the aforementioned methods, wherein the inflammatory condition is selected from the group consisting of osteoarthritis, rheumatoid arthritis, psoriatic arthritis, gout, tendinitis, bursitis, and ankylosing spondylitis.

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

For purposes of this invention, the chemical elements are identified in accordance with the Periodic Table of the Elements, CAS version, Handbook of Chemistry and Physics, 67th Ed., 1986-87, inside cover.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### D-Amino Acid Hydrogelators

### Experimental Materials and Instruments.

All of the solvents and chemical reagents were used as received from the commercial sources without further purification unless otherwise noted. Flash chromatography was performed on silica gel 60 (230-400 mesh). Analytical thin layer chromatography (TLC) was performed using silica gel 60 F-254 pre-coated glass plates (0.25 mm) and analyzed by short wave UV illumination. Hydrophilic products were purified with Waters Delta600 HPLC system, which equipped with an XTerra C18 RP column and an in-line diode array UV detector. ¹H, ¹³C, and ³¹P NMR spectra were obtained on Varian Unity Inova 400. Chemical shifts are reported in δ (ppm) relative to the solvent residual peak (phosphoric acid for ³¹P NMR). Coupling constants are reported in Hz with multiplicities denoted as s (singlet), d (doublet), t (triplet), q (quartet), p (pentet), m (multiplet) and br (broad). LC-MS spectra were obtained on a Waters Acouity ultra Performance LC with Waters MICRO-MASS detector. Rheological data were measured on TA ARES G2 rheometer with 25 mm cone plate. TEM images were taken on Morgagni 268 transmission electron microscope. The PTP1B inhibitor was purchased from BIOMOL. The HeLa cell line (CCL2) was purchased from American Type Culture Collection. All of the media were purchased from Invitrogen. Cytotoxicity tests were measured by DTX 880 multimode detector.

### Synthesis and Characterizations

### 2-(Naphthalen-2-yl)acetyl-(L)-Phe-(L)-Phe-(L)-Lys-(L)-Tyr phosphate (11a)

The L-amino acid based hydrogelator precursor was prepared by the standard solid-phase peptide synthesis (SPPS), which used 2-chlorotrityl chloride resin (100˜200 mesh and 0.3˜0.8 mmol/g) and N-Fmoc-protected amino acids with side chains properly protected by tert-butoxycarbonyl (Fmoc-Lys(Boc)-OH) group. Fmoc-Tyr(PO₃H₂)—OH was prepared from L-Tyr-OH and directly used in SPPS. Ottinger, E. A.; et al. Biochemistry 1993, 32, 4354. The resin was first swelled in dry dichloromethane (DCM) by bubbling it with nitrogen gas (N₂) for 20 minutes, and was washed with 3 mL of dry N,N-dimethylformamide (DMF) for three times. Then the first amino acid Fmoc-Tyr(PO₃H₂)—OH was loaded onto resin at its C-terminal by bubbling the resin in a DMF solution of Fmoc-protected amino acid (2 equiv.) and 1 mL of N,N-diisopropylethylamine (DIPEA) for 1 hour. After washed with 3 mL of DMF for three times, the unreacted sites in resin were quenched by bubbling the resin with blocking solution (16:3:1 of DCM/MeOH/DIPEA) for 2×10 minutes. Then the resins were treated with 20% piperidine (in DMF) for 0.5 hour to remove the protecting group, followed by washing the resin in DMF for five times. Then we conjugated the sequent Fmoc-protected amino acid (2 equiv.) to the free amino group on the resin using DIPEA/O-benzotriazole-N,N,N′,N′-tetramethyl-uroniumhexafluoro-phosphate (HBTU) (2 equiv.) as the coupling reagent. These coupling and deprotection steps were repeated to elongate the peptide chain, which were carried out by the standard Fmoc SPPS protocol. Chan, W. C.; White, P. Fmoc Solid Phase Peptide Synthesis: A Practical Approach; OUP Oxford, 2000. The resin was washed with DMF for 3˜5 times after each step. Finally, we washed the resin with DMF (5 times), DCM (5 times), methanol (5 times), and hexane (5 times) respectively, then we cleaved the peptide with TFA (10 mL) for 2 hours. The resulted crude products were purified by reverse phase HPLC and gave a total yield of 64%. ¹H NMR (400 MHz, DMSO-d₆) δ 8.35 (d, J=8.3 Hz, 1H), 8.24 (d, J=8.1 Hz, 1H), 8.12 (d, J=7.8 Hz, 1H), 8.04 (s, 3H), 7.93 (d, J=7.5 Hz, 1H), 7.85 (d, J=7.4 Hz, 1H), 7.78 (d, J=7.5 Hz, 1H), 7.74 (d, J=8.4 Hz, 1H), 7.58 (s, 1H), 7.51-7.41 (m, 2H), 7.32-7.00 (m, 15H), 4.60-4.45 (m, 2H), 4.45-4.37 (m, 1H), 4.16 (dd, J=14.6, 7.0 Hz, 1H), 3.53 (dd, J=35.9, 14.0 Hz, 2H), 3.13-3.02 (m, 2H), 2.97-2.87 (m, 2H), 2.80 (dd, J=13.9, 9.6 Hz, 1H), 2.69 (dd, J=13.4, 10.3 Hz, 1H), 2.54 (s, 2H), 1.46-0.95 (m, 6H); ¹³C NMR (101 MHz, DMSO-d₆): δ 172.79, 171.16, 171.03, 170.54, 169.84, 137.83, 137.72, 133.91, 132.90, 131.74, 129.73, 129.27, 129.16, 128.03, 127.90, 127.61, 127.44, 127.38, 127.25, 126.29, 126.14, 125.97, 125.42, 119.25, 119.14, 53.94, 53.69, 53.06, 52.92, 42.25, 37.54, 37.20, 35.53, 31.97, 26.62, 22.01; 31P NMR (162 MHz, DMSO-d₆) δ −4.84; LC-MS (ESI) (m/z): C₄₅H₅₀N₅O₁₀P calcd 851.33. found 852.64 [M+1]′, 850.69 [M−1]⁻.

### 2-(Naphthalen-2-yl)acetyl-(D)-Phe-(D)-Phe-(D)-Lys-(D)-Tyr phosphate (11b)

The D-amino acid based hydrogelator precursor was also synthesized by solid-phase peptide synthesis described as above. All the N-Fmoc-protected amino acids we used here were D-version amino acids, including Fmoc-D-Phe-OH, Fmoc-D-Lys(Boc)-OH, and Fmoc-D-Tyr(PO₃H₂)—OH. Fmoc-DTyr(PO₃H₂)—OH was also prepared from D-Tyr-OH and directly used in SPPS. Purification with reverse phase HPLC gave pure white powder in a yield of 57%. ¹H NMR (400 MHz, DMSO-d₆) δ 8.37 (d, J=7.8 Hz, 1H), 8.26 (d, J=6.7 Hz, 1H), 8.16 (s, 4H), 7.91 (d, J=5.3 Hz, 1H), 7.85 (d, J=5.9 Hz, 1H), 7.78 (d, J=6.3 Hz, 1H), 7.74 (d, J=8.2 Hz, 1H), 7.59 (s, 1H), 7.46 (s, 2H), 7.29-7.03 (m, 15H), 4.61-4.46 (m, 2H), 4.42 (s, 1H), 4.13 (s, 1H), 3.53 (dd, J=35.9, 13.9 Hz, 2H), 3.08 (d, J=12.4 Hz, 2H), 2.93 (d, J=11.7 Hz, 2H), 2.87-2.76 (m, 1H), 2.75-2.64 (m, 1H), 1.47-0.91 (m, 6H); ¹³C NMR (101 MHz, DMSO-d₆) δ 172.88, 171.19, 171.06, 170.58, 169.82, 152.26, 137.81, 137.76, 133.94, 132.93, 131.73, 130.64, 129.69, 129.30, 129.27, 128.07, 127.94, 127.65, 127.49, 127.43, 127.31, 127.24, 126.25, 126.17, 126.00, 125.48, 119.23, 119.19, 53.92, 53.73, 53.16, 52.96, 42.26, 38.55, 37.59, 37.23, 35.45, 32.02, 26.65, 22.06; ³¹P NMR (162 MHz, DMSO-d₆) δ −4.87; LC-MS (ESI) (m/z): C₄₅H₅₀N₅O₁₀P calcd 851.33. found 852.64 [M+1]⁺, 850.69 [M−1]⁻.

### 2-(Naphthalen-2-yl)acetyl-(D)-Phe-(D)-Phe-(D)-Lys(NBD)-(D)-Tyr phosphate (14b)

112.0 mg (0.13 mmol) of hydrogelator precursor 11b was dissolved in an aqueous solution with pH adjusted to 9.0 by Na₂CO₃. Then the methanol solution of 26.0 mg (0.13 mmol) of 4-chloro-7-nitro-2,1,3-benzoxadiazole was added into the above solution of 11b dropwise and the resulting solution was stirred for 2 h at 50 C. After cooled down the solution, we neutralized it with 1 M HCl, followed by the purification with HPLC (detected at 430 nm). Pure orange powder was collected in a yield of 48%. ¹H NMR (400 MHz, DMSO-d₆) δ 9.59 (s, 1H), 8.45 (d, J=8.0 Hz, 1H), 8.26 (d, J=7.2 Hz, 1H), 8.16 (d, J=7.9 Hz, 1H), 8.09 (d, J=7.7 Hz, 1H), 7.82 (d, J=7.8 Hz, 1H), 7.74 (dd, J=16.1, 8.2 Hz, 2H), 7.58 (s, 1H), 7.50-7.38 (m, 2H), 7.28-7.01 (m, 15H), 6.33 (d, J=9.1 Hz, 1H), 4.53 (s, 1H), 4.47 (s, 1H), 4.43-4.29 (m, 2H), 3.53 (dd, J=34.5, 14.2 Hz, 2H), 3.39 (s, 2H), 3.06-2.63 (m, 6H), 1.78-1.28 (m, 6H); ¹³C NMR (101 MHz, DMSO-d₆) δ 172.81, 171.45, 171.20, 170.68, 169.79, 151.46, 145.11, 144.42, 144.16, 138.05, 137.77, 133.95, 132.90, 131.69, 129.86, 129.26, 127.94, 127.40, 126.15, 125.94, 125.42, 120.49, 119.69, 119.60, 99.12, 53.97, 53.77, 53.66, 52.31, 43.37, 42.26, 37.64, 37.32, 35.91, 31.93, 27.32, 22.62; ³¹P NMR (162 MHz, DMSO-d₆) δ −5.17; LC-MS (ESI) (m/z): C₅₁H₅₁N₈O₁₃P calcd 1014.33. found 1015.56 [M+1]⁺, 1013.67 [M−1]⁻.

### 2′-NHS-succinyl-paclitaxel (18)

69.6 mg (0.70 mmol) of succinic anhydride and 45.8 mg (0.37 mmol) of 4-dimethylaminopyridine were added to a solution of 170.8 mg (0.2 mmol) of paclitaxel (16) in 5 mL of dry pyridine. After stirred for 3 hours at 20° C., the mixture was extracted with 20 mL of dry dichloromethane (DCM) and 1 M HCl solution (20 mL×3). Then the organic phase was washed with water (20 mL×3) and brine (10 mL×3), followed by the treatment with anhydrous sodium sulfate and the evaporation under reduced pressure. The residue of 2′-succinyl-paclitaxel (17) was then dissolved in 5 mL of chloroform and reacted with 23.0 mg (0.20 mmol) of N-hydroxysuccinimide (NHS) and 27.8 mg (0.22 mmol) of N,N′-diisopropylcarbodiimide (DIC) without further purification. After stirred for 6 h at 20° C., the resulting mixture was filtered to remove N,N′-diisopropylurea (DIU) and the filtrate was concentrated by rotary evaporator. Purification with column chromatography over silica gel (1:0-20:1 dichloromethane/methanol) gave pure white product of 2′-NHS-succinyl-paclitaxel (18) with yield of 92%. ¹H NMR (400 MHz, CDCl₃) δ 8.14 (d, J=7.2 Hz, 2H), 7.74 (d, J=7.1 Hz, 2H), 7.61 (t, J=7.4 Hz, 1H), 7.56-7.45 (m, 3H), 7.45-7.29 (m, 7H), 7.16 (d, J=9.2 Hz, 1H), 6.28 (s, 1H), 6.22 (t, J=8.6 Hz, 1H), 5.98 (dd, J=9.1, 3.6 Hz, 1H), 5.68 (d, J=7.1 Hz, 1H), 5.52 (d, J=3.6 Hz, 1H), 5.15 (s, 1H), 4.97 (d, J=8.0 Hz, 1H), 4.44 (dd, J=10.5, 6.7 Hz, 1H), 4.31 (d, J=8.5 Hz, 1H), 4.20 (d, J=8.3 Hz, 1H), 3.80 (d, J=7.0 Hz, 1H), 3.02-2.80 (m, 4H), 2.73 (s, 4H), 2.61-2.50 (m, 1H), 2.43 (s, 3H), 2.34 (dd, J=15.4, 9.4 Hz, 1H), 2.23 (s, 3H), 2.13 (dd, J=15.2, 8.9 Hz, 1H), 1.91 (s, 3H), 1.90-1.83 (m, 1H) 1.67 (s, 3H), 1.22 (s, 3H), 1.13 (s, 3H).

### 2-(Naphthalen-2-yl)acetyl-(D)-Phe-(D)-Phe-(D)-Lys(Taxol)-(D)-Tyr phosphate (19b)

170.2 mg (0.20 mmol) of 11b was dissolved in 3 mL of water with carefully adding Na2CO3 (1.5 equiv.) to adjust the pH of aqueous solution to 8.0. Then the acetone solution (2 mL) of 198.2 mg (0.18 mmol) of 2′-NHS-succinyl-paclitaxel (18) added into the weak basic aqueous solution of 11b dropwise. More acetone and water were added carefully to keep the resulting solution clear. After the mixture was stirred at 20° C. overnight, it was purified with HPLC and gave pure white powder in a yield of 37%. ¹H NMR (400 MHz, DMSO-d₆) δ 9.22 (d, J=8.2 Hz, 1H), 8.28 (d, J=8.2 Hz, 1H), 8.22-8.13 (m, 2H), 8.07 (dd, J=15.5, 7.6 Hz, 1H), 8.02-7.93 (m, 2H), 7.89-7.81 (m, 4H), 7.80-7.69 (m, 3H), 7.69-7.62 (m, 2H), 7.61-7.52 (m, 3H), 7.52-7.38 (m, 8H), 7.26-7.10 (m, 13H), 7.07 (d, J=7.8 Hz, 2H), 6.29 (s, 1H), 5.82 (t, J=8.9 Hz, 1H), 5.53 (t, J=8.6 Hz, 1H), 5.41 (d, J=7.0 Hz, 1H), 5.34 (d, J=8.8 Hz, 1H), 4.91 (d, J=9.4 Hz, 1H), 4.65-4.47 (m, 3H), 4.42 (dd, J=13.0, 7.5 Hz, 1H), 4.31 (dd, J=13.1, 8.2 Hz, 1H), 4.16-4.07 (m, 1H), 4.01 (t, J=10.4 Hz, 2H), 3.57 (d, J=13.1 Hz, 2H), 3.48 (dd, J=13.7, 5.7 Hz, 1H), 3.08-2.64 (m, 8H), 2.59 (s, 2H), 2.44-2.28 (m, 4H), 2.23 (s, 3H), 2.19-2.11 (m, 1H), 2.09 (s, 3H), 1.77 (s, 3H), 1.72-1.56 (m, 3H), 1.50 (s, 3H), 1.41-1.16 (m, 6H), 1.02 (s, 3H), 0.99 (s, 3H); ¹³C NMR (101 MHz, DMSO-d₆) δ 202.39, 172.70, 172.04, 171.58, 171.14, 170.68, 170.01, 169.76, 169.68, 169.17, 168.80, 166.43, 165.22, 150.13, 139.45, 137.78, 137.62, 137.38, 134.26, 133.89, 133.53, 133.35, 132.90, 131.71, 131.51, 130.21, 130.14, 129.94, 129.60, 129.23, 128.71, 128.34, 128.21, 128.01, 127.91, 127.68, 127.58, 127.46, 127.36, 127.26, 127.22, 126.24, 126.15, 125.97, 125.45, 119.83, 119.78, 83.56, 80.26, 76.74, 75.32, 74.57, 70.72, 70.45, 57.40, 54.03, 53.74, 53.68, 53.54, 52.31, 46.10, 42.95, 42.21, 38.63, 37.52, 37.40, 36.53, 35.89, 34.40, 32.03, 29.54, 28.91, 28.77, 26.36, 22.72, 22.58, 21.42, 20.72, 13.95, 9.81; ³¹P NMR (162 MHz, DMSO-d₆) δ −5.16; LC-MS (ESI) (m/z): C₉₆H₁₀₃N₆O₂₆P calcd 1786.67. found 1788.80 [M+1]′, 1786.01 [M−1]⁻.

### Characterization of the Properties of Self-Assembly.

General Procedure for Hydrogel Preparation.

All the compounds were dissolved in de-ionized water. We then adjusted pH of the solutions carefully adding 1 M of NaOH and 1 M of HCl and measured the values by pH paper (pH 6.0-8.0). After prepared clear weakly basic solutions (pH 7.6 or 7.4), we then formed the hydrogels by adding enzymes (alkaline phosphatase).

TEM Sample Preparation.

For this example, we used a negative staining technique to study the TEM images. The 400 mesh copper grids coated with continuous thick carbon film (˜35 nm) were first glowed discharge just before use to increase their hydrophilicity. After the sample solution (3 μL) was placed onto the grid (sufficient volume to cover the grid surface), we then rinsed grid with dd-H₂O for three times. In this rinsing step, we first let the grid touch the water drop with the sample-loaded surface facing the parafilm, then gently absorb water from the edge of the grid with the aid of a filter paper sliver. Immediately after rinsing, the grid was stained by UA stain solution (2.0% (w/v) uranyl acetate) for three times. Similar to the rinsing step, we first let the grid touch the stain solution drop with the sample-loaded surface facing the parafilm, then gently absorb the redundant stain solution from the edge of the grid using a filter paper sliver. Then we allow the grid to dry in air and examine the grid as soon as possible.

Rheological Measurement.

Rheological tests were conducted on TA ARES G2 rheometer with 25 mm cone-plate and TA Orchestrator Software during the experiment. The minimum volume of hydrogel sample placed on the cone-plate was 0.2 mL. Here we perform both dynamic strain sweep and dynamic frequency sweep on our hydrogels

(1) Dynamic strain sweep: The measurement was performed at the frequency of 6.28 rad/s and temperature at 25° C. Carried out with the “log” sweep mode, we applied strain to the hydrogel sample from 0.1 to 100% (10 points per decade). The critical strain (γ_{c}) value was determined from the storage-strain profiles of the hydrogel sample. Over a certain strain, a drop in the elastic modulus was observed. Then we determined the strain amplitude (γ_{c}) at which storage moduli (G′) just begins to decrease by 5% from its maximum value, which was taken as a measure of the critical strain of the hydrogels and corresponded to the breakdown of the cross-linked network in the hydrogel samples.
(2) Dynamic frequency sweep: The frequency ranged from 200 rad/s to 0.1 rad/s, depending on the viscoelastic properties of each sample. A suitable strain, which was the average value around maximum storage moduli during dynamic strain sweep, was used to ensure the linearity of dynamic viscoelasticity.

### Biological Applications and the In Vivo Tests

MTT Assays for Cytotoxicity.

We seeded 5×10⁵ (cells/well) of health HeLa cells into 96-well plate with 100 μL of MEM medium supplemented with 10% fetal bovine serum (FBS), 100 U/mL penicillin and 100 mg mL21 streptomycin. The incubation at 37° C. and 5% CO₂ for 12 hours allowed HeLa cells to attach the bottom of 96-well plate. Then we replaced the medium by another 100 μL of growth medium that contained serial diluents of our compounds (0.5% DMSO) and then incubated the cells at 37° C. and 5% CO₂ for additional 72 hours. During the measurement of proliferation for HeLa cells, which were assayed into three days, we added 10 μL of (3-(4,5-Dimethylthiazol-2-yl)-2,5-diphenyltetrazolium bromide (MTT, 0.5 mg/mL) into the assigned wells in their corresponding day every 24 hours, which was followed by adding 100 μL of 0.1% sodium dodecyl sulfate (SDS) 4 hours later. Then we collected the assay results after another 24 hours incubation. Since the mitochondrial reductase in living cells reduced MTT to purple fomazan, the absorbance at 595 nm of the whole solution was finally measured by DTX 880 Multimode Detector. With MEM medium as blank and untreated HeLa cells as control, we measure each concentration of these compounds in triplicate. The IC₅₀ values of our hydrogelators were read from their activity curves (with the measurement of 8 different concentrations) in day 3.

Live Cell Imaging.

We seeded 2×10⁵ of HeLa cells in Glass Chamber (Thermo Scientific Nunc Lab-Tek, 2-well) with MEM medium (2 mL) that was supplemented with 10% FBS, 100 U/mL penicillin and 100 mg mL 21 streptomycin for 4 h to allow the cell attachment. In order to perform the PTP1B Inhibition assay, we then replaced the culture medium in both wells and re-incubated the cells for 1 h, for which one-half of the wells was incubated in the medium containing 25 μM CinnGEL 2Me (novel inhibitor of PTP1B, prepared by reconstitution in DMSO), other well as a control was in the culture medium plus the same volume of DMSO. Ishino, Y.; et al. Mol. Vis. 2008, 14, 61. After that, we replaced the medium and washed the HeLa cells with PBS buffer for three times. Then we fixed the cell containing glass chamber on the confocal microscope stage, and replaced the PBS buffer with 1 mL solution of our fluorescent hydrogelators (500 μM, dissolved in PBS buffer) for each well. The sample we added into the PTP1B inhibition well also contained 25 μM of CinnGEL 2Me. Thereafter fluorescent images were captured immediately in the xyt mode with a delay of 11.64 s between frames.

In Vivo Evaluation of Antitumor Activity.

Female Balb/c mice were incubated with 2×10⁵ 4 T1-luciferase cells in the mammary fat pad. Tumor growth was monitored every other day and the tumor volume was calculated by the formula: length×width×(Length+Width)/2. Once tumors size reached around 500 mm³, we randomly divided mice into different treatment groups. (a) 4×10 mg/kg of Taxol formulated with Cremophor EL was intravenous injected (I.V.) every other day from day 0 (the day giving drugs) for indicated times; (b) 10 mg/kg of our hydrogel in 40 μL volume was intratumoral injected at day 0; (c) the PBS vehicle control was intratumoral injected at day 0. Mice died immediately with injecting 40 mg/kg of Taxol in one injection due to its cytotoxicity. The Taxol containing hydrogels 20a and 20b were prepared by enzyme treatment in PBS buffer (pH 7.4) before their intratumoral injections, which could sustain one month. Mice weight was monitored after receiving treatment and presented as relative weight (%).

Biostability Test in the Presence of Proteinase K.

1 mg of 11a and 11b were dissolved in 5 mL of HEPES buffer at pH 7.5, respectively. Then 3.2 U/mL of proteinase K were added into both solutions, which followed by incubation at 37° C. for 24 h. 50 μL of sample was taken out at 1, 2, 4, 8, 12, and 24 h and analyzed by HPLC.

### Example 2

### NSAID Hydroelators

### Materials and Instruments.

All of the chemical reagents and solvents were used as received from the commercial sources without further purification unless otherwise noted. ¹H, ¹³C, and ³¹P NMR spectra were obtained on Varian Unity Inova 400. Chemical shifts are reported in δ (ppm) relative to the solvent residual peak (phosphoric acid for ³¹P NMR). Coupling constants are reported in Hz with multiplicities denoted as s (singlet), d (doublet), t (triplet), q (quartet), p (pentet), m (multiplet) and br (broad). The HeLa cell line (CCL2) was purchased from American Type Culture Collection. All of the medium were provided from Invitrogen. COX inhibitor screening assay kit (700100) was purchased from Cayman Chemical Company. Cytotoxicity test and COX inhibition tests were measured by DTX 880 Multimode Detector. Rheological data were measured on TA ARES G2 rheometer with 25 mm cone plate. TEM images were taken on Morgagni 268 transmission electron microscope. LC-MS was performed on a Waters Acouity ultra Performance LC with Waters MICRO-MASS detector.

### Synthesis and Characterizations.

Solid-Phase Peptide Synthesis (SPPS).

All the hydrogelators were prepared by solid-phase peptide synthesis (SPPS) using 2-chlorotrityl chloride resin (100˜200 mesh and 0.3˜0.8 mmol/g) and N-Fmoc-protected amino acids with side chains properly protected by a tert-butyl (Fmoc-D-Tyr(tBu)-OH) or tert-butoxycarbonyl (Fmoc-D-Lys(Boc)-OH) group. Bubbled with nitrogen gas (N₂) in dry dichloromethane (DCM) for 20 minutes, the resin swelled and was washed with dry N,N-dimethylformamide (DMF) (3×3 mL). Then the first amino acid was loaded onto resin at its C-terminal by bubbling the resin in a DMF solution of Fmoc-protected amino acid (2 equiv.) and N,N-diisopropylethylamine (DIPEA) for 0.5 hour. After washed with DMF (3×3 mL), the resin was bubbled with the blocking solution (16:3:1 of DCM/MeOH/DIPEA) for 0.5 hour to deactivate the unreacted sites. Then the resins were treated with 20% piperidine (in DMF) for 0.5 hour to remove the protecting group, followed by coupling Fmoc-protected amino acid (2 equiv.) to the free amino group on the resin using DIPEA/O-benzotriazole-N,N,N′,N′-tetramethyl-uronium-hexafluoro-phosphate (HBTU) (2 equiv.) as the coupling reagent. These two steps were repeated to elongate the peptide chain, which were carried out by the standard Fmoc SPPS protocol. The resin was washed with DMF for 3-5 times after each step. At the final step, the peptide was cleaved with TFA (10 mL) for 2 hours and the resulted crude products were purified by reverse phase HPLC. For phosphate containing hydrogelators 2 and 4, Fmoc-D-Try(PO₃H₂)—OH was prepared from D-Try and directly used in SPPS, which need longer coupling reaction time (1 hour).

N-Hydroxysuccinimide Assisted Coupling Reaction.

In addition to the solid-phase peptide synthesis, N-hydroxysuccinimide (NHS) assisted coupling reaction was also performed in the preparation of hydrogelators 5 and 6. 115 mg (1.0 mmol) of N-hydroxysuccinimide (NHS) and 152 mg (1.2 mmol) of N,N′-diisopropylcarbodiimide (DIC) were added to a solution of 230 mg (1.0 mmol) of naproxen (Npx) in chloroform (10 mL). After the resulting mixture was stirred for 2 h at room temperature, filtration, evaporation, and recrystallization in ethanol were performed to give pure Npx-NHS ester. To an aqueous solution (6 mL) dissolving 405 mg (1.0 mmol) of the Fmoc-D-Lys-OH (pH was adjusted to 8.5 by Na₂CO₃), the acetone solution of Npx-NHS ester (6 mL) was added dropwise, and the resulting solution was stirred overnight at room temperature. The solution was concentrated by rotary evaporator until all the acetone was removed. 1 M of HCl was added to adjust the pH of the remaining aqueous solution to 3.0 and the resulting white precipitate was collected by filtration, followed by the purification with flash column. Then the pure product, Fmoc-D-Lys(Npx)-OH, was directly used in standard SPPS (described as above) with longer coupling reaction time (1 hour).

### Naproxen-D-Phe-D-Phe (1)

¹H NMR (400 MHz, DMSO-d₆) δ 8.27 (d, J=7.7 Hz, 1H), 8.06 (d, J=8.3 Hz, 1H), 7.71 (d, J=9.0 Hz, 1H), 7.63 (d, J=8.5 Hz, 1H), 7.57 (s, 1H), 7.31-7.18 (m, 7H), 7.13 (dd, J=8.9, 2.3 Hz, 1H), 7.04-6.88 (m, 5H), 4.57-4.42 (m, 2H), 3.86 (s, 3H), 3.77 (q, J=6.8 Hz, 1H), 3.15-2.84 (m, 3H), 2.70 (dd, J=13.8, 9.7 Hz, 1H), 1.33 (d, J=6.9 Hz, 3H); ¹³C NMR (101 MHz, DMSO-d₆) δ 173.03, 172.73, 171.13, 156.92, 137.44, 137.42, 136.91, 133.06, 129.16 (2C), 129.12 (2C), 129.07, 128.29, 128.19 (2C), 127.65 (2C), 126.45 (2C), 126.38, 125.92, 125.22, 118.37, 105.61, 55.14, 53.52, 53.45, 44.45, 37.38, 36.72, 17.81.

### Naproxen-D-Phe-D-Phe-D-Tyr phosphate (2)

¹H NMR (400 MHz, DMSO-d₆) δ 8.36 (d, J=7.5 Hz, 1H), 8.14 (d, J=8.1 Hz, 1H), 8.05 (d, J=8.3 Hz, 1H), 7.71 (d, J=9.0 Hz, 1H), 7.63 (d, J=8.5 Hz, 1H), 7.57 (s, 1H), 7.30-7.05 (m, 12H), 6.97-6.82 (m, 5H), 4.59 (dd, J=12.7, 8.7 Hz, 1H), 4.51-4.40 (m, 2H), 3.86 (s, 3H), 3.77 (q, J=6.8 Hz, 1H), 3.04 (dd, J=13.8, 4.3 Hz, 2H), 2.93 (dd, J=13.8, 8.1 Hz, 1H), 2.89-2.75 (m, 2H), 2.67 (dd, J=13.5, 9.9 Hz, 1H), 1.32 (d, J=6.9 Hz, 3H); ¹³C NMR (101 MHz, DMSO-d₆) δ 173.05, 172.59, 171.08, 170.91, 156.92, 150.37, 137.61, 137.47, 136.95, 133.07, 132.57, 130.10 (2C), 129.30 (2C), 129.12 (3C), 128.30, 128.02 (2C), 127.61 (2C), 126.46 (2C), 126.28, 125.87, 125.28, 119.82, 119.78, 118.37, 105.61, 55.14, 53.66, 53.60, 53.55, 44.43, 37.62, 37.30, 35.97, 17.84; ³¹P NMR (162 MHz, DMSO-d₆) δ −5.15.

### Naproxen-D-Phe-D-Phe-D-Lys (3)

¹H NMR (400 MHz, DMSO-d₆) δ 8.33-7.96 (m, 3H), 7.70 (d, J=8.6 Hz, 1H), 7.63 (d, J=8.0 Hz, 1H), 7.56 (s, 1H), 7.36-7.04 (m, 8H), 7.04-6.66 (m, 5H), 4.58 (s, 1H), 4.43 (s, 1H), 4.15 (s, 1H), 3.86 (s, 3H), 3.77 (s, 1H), 3.04 (dd, J=31.1, 11.0 Hz, 2H), 2.92-2.61 (m, 4H), 1.88-1.44 (m, 4H), 1.42-1.08 (m, 5H); ¹³C NMR (101 MHz, DMSO-d₆) δ 173.15, 173.07, 170.96, 170.66, 156.93, 137.60, 137.53, 136.85, 133.07, 129.31 (2C), 129.04 (3C), 128.29, 128.03 (2C), 127.66 (2C), 126.41 (2C), 126.29, 125.91, 125.23, 118.39, 105.63, 55.14, 53.78, 53.65, 52.16, 44.49, 38.64, 37.58, 37.25, 30.80, 26.63, 22.27, 17.84.

### Naproxen-D-Phe-D-Phe-D-Lys-D-Tyr phosphate (4)

¹H NMR (400 MHz, DMSO-d₆) δ 8.20-8.04 (m, 4H), 7.88 (d, J=8.1 Hz, 1H), 7.71 (d, J=9.0 Hz, 1H), 7.63 (d, J=8.6 Hz, 1H), 7.57 (s, 1H), 7.31-7.02 (m, 12H), 7.01-6.83 (m, 5H), 4.56 (dd, J=12.4, 8.6 Hz, 1H), 4.42 (dd, J=12.7, 9.0 Hz, 2H), 4.14 (dd, J=14.2, 6.9 Hz, 1H), 3.86 (s, 3H), 3.78 (d, J=7.0 Hz, 1H), 3.09 (d, J=13.7 Hz, 2H), 2.92 (dd, J=13.7, 10.6 Hz, 1H), 2.86-2.77 (m, 2H), 2.66 (dd, J=13.7, 9.7 Hz, 1H), 2.54 (s, 2H), 1.47-1.28 (m, 7H), 1.11-0.93 (m, 2H); ¹³C NMR (101 MHz, DMSO-d₆) δ 173.10, 172.78, 171.00, 170.97, 170.54, 156.92, 152.20, 137.71, 137.47, 136.91, 133.07, 130.66, 129.66 (2C), 129.29 (2C), 129.07 (3C), 128.29, 128.03 (2C), 127.63 (2C), 126.42 (2C), 126.24, 125.89, 125.28, 119.20, 119.16, 118.38, 105.63, 55.14, 53.75, 53.58, 53.07, 52.90, 44.49, 38.51, 37.32 (2C), 35.41, 31.97, 26.59, 22.01, 17.86; ³¹P NMR (162 MHz, DMSO-d₆) δ −5.19.

### D-Phe-D-Phe-D-Lys(naproxen) (5)

¹H NMR (400 MHz, DMSO-d₆) δ 8.71 (d, J=7.8 Hz, 1H), 8.41 (d, J=7.6 Hz, 1H), 8.00 (t, J=5.3 Hz, 1H), 7.76 (d, J=9.1 Hz, 1H), 7.73 (d, J=8.7 Hz, 1H), 7.69 (s, 1H), 7.43 (d, J=8.6 Hz, 1H), 7.33-7.16 (m, 11H), 7.13 (dd, J=8.9, 2.4 Hz, 1H), 4.65 (dd, J=12.5, 8.5 Hz, 1H), 4.18 (dd, J=13.0, 8.1 Hz, 1H), 3.94 (s, 1H), 3.85 (s, 3H), 3.71 (dd, J=13.9, 6.9 Hz, 1H), 3.15-2.93 (m, 4H), 2.92-2.78 (m, 2H), 1.73 (dd, J=13.3, 5.6 Hz, 1H), 1.59 (dd, J=13.8, 7.1 Hz, 1H), 1.39 (d, J=7.0 Hz, 5H), 1.30 (dd, J=14.0, 6.6 Hz, 2H); ¹³C NMR (101 MHz, DMSO-d₆) δ 173.40, 173.23, 170.66, 168.55, 156.96, 137.53, 137.43, 135.07, 133.10, 129.57 (2C), 129.28 (3C), 129.06, 128.41, 128.37, 128.12 (2C), 127.00, 126.56, 126.45, 126.40, 125.20, 118.54, 105.68, 55.15, 53.92, 53.37, 51.94, 45.07, 38.42, 37.63, 37.30, 30.74, 28.75, 22.85, 18.63.

### D-Phe-D-Phe-D-Lys(naproxen)-D-Tyr (6)

¹H NMR (400 MHz, DMSO-d₆) δ 9.22 (s, 1H), 8.70 (d, J=7.9 Hz, 1H), 8.25 (d, J=8.0 Hz, 1H), 8.10 (d, J=7.4 Hz, 1H), 7.96 (d, J=5.2 Hz, 1H), 7.75 (d, J=9.4 Hz, 1H), 7.73 (d, J=10.8 Hz, 1H), 7.69 (s, 1H), 7.43 (d, J=8.4 Hz, 1H), 7.34-7.08 (m, 12H), 7.01 (d, J=8.2 Hz, 2H), 6.65 (d, J=8.1 Hz, 2H), 4.68-4.59 (m, 1H), 4.40-4.26 (m, 2H), 3.97 (s, 1H), 3.85 (s, 3H), 3.70 (d, J=6.7 Hz, 1H), 3.16-2.66 (m, 8H), 1.72-1.57 (m, 1H), 1.57-1.44 (m, 1H), 1.38 (d, J=6.8 Hz, 5H), 1.31-1.14 (m, 2H); ¹³C NMR (101 MHz, DMSO-d₆) δ 173.20, 172.86, 171.35, 170.32, 168.07, 156.96, 155.95, 137.53, 137.48, 134.72, 133.09, 130.02 (2C), 129.60 (2C), 129.23 (2C), 129.07, 128.45 (2C), 128.36, 128.10 (2C), 127.36, 127.11, 126.57, 126.45, 126.35, 125.20, 118.56, 114.97 (2C), 105.67, 55.16, 53.99, 53.76, 53.10, 52.34, 45.06, 38.63, 37.57, 37.02, 35.89, 32.03, 28.91, 22.63, 18.67.

### Naproxen-L-Phe-L-Phe (L-1)

¹H NMR (400 MHz, DMSO-d₆) δ 8.24 (d, J=7.5 Hz, 1H), 8.11 (d, J=8.4 Hz, 1H), 7.72 (d, J=9.1 Hz, 1H), 7.69 (d, J=8.8 Hz, 1H), 7.63 (s, 1H), 7.34 (d, J=8.3 Hz, 1H), 7.30-7.03 (m, 12H), 4.60 (s, 1H), 4.39 (dd, J=13.1, 7.2 Hz, 1H), 3.85 (s, 3H), 3.74 (q, J=6.8 Hz, 1H), 3.08-2.92 (m, 2H), 2.92-2.69 (m, 2H), 1.20 (d, J=6.6 Hz, 3H); ¹³C NMR (101 MHz, DMSO-d₆) δ 173.07, 172.65, 171.13, 156.93, 137.79, 137.24, 137.01, 133.08, 129.32 (2C), 129.07, 128.99 (2C), 128.31, 128.10 (2C), 127.88 (2C), 126.61, 126.44, 126.33, 126.16, 125.35, 118.43, 105.64, 55.56, 53.13, 53.33, 44.67, 37.74, 36.65, 18.69.

### Naproxen-L-Phe-L-Phe-L-Tyr (L-2)

1H NMR (400 MHz, DMSO-d₆) δ 8.17-8.00 (m, 3H), 7.72 (d, J=9.0 Hz, 1H), 7.68 (d, J=8.6 Hz, 1H), 7.62 (s, 1H), 7.33 (d, J=8.5 Hz, 1H), 7.29-6.96 (m, 14H), 6.64 (d, J=8.3 Hz, 2H), 4.59-4.51 (m, 1H), 4.50-4.41 (m, 1H), 4.31 (dd, J=12.9, 6.9 Hz, 1H), 3.84 (s, 3H), 3.72 (d, J=6.9 Hz, 1H), 3.04-2.90 (m, 3H), 2.83 (dd, J=13.8, 7.5 Hz, 1H), 2.78-2.64 (m, 2H), 1.18 (d, J=7.0 Hz, 3H); ¹³C NMR (101 MHz, DMSO-d₆) δ 173.14, 173.07, 170.89, 170.51, 156.92, 155.85, 137.93, 137.56, 137.02, 133.08, 130.16 (2C), 129.33 (2C), 129.10 (3C), 128.33, 127.90 (2C), 127.85 (2C), 127.71, 126.62, 126.45, 126.09, 126.07, 125.36, 118.41, 114.91 (2C), 105.63, 55.13, 54.27, 53.81, 53.48, 44.68, 37.69, 37.55, 36.11, 18.72.

### Naproxen-L-Phe-L-Phe-L-Lys (L-3)

¹H NMR (400 MHz, DMSO-d₆) δ 8.24 (d, J=7.7 Hz, 1H), 8.11 (d, J=8.6 Hz, 1H), 8.02 (d, J=8.1 Hz, 1H), 7.74 (d, J=9.0 Hz, 1H), 7.69 (d, J=8.6 Hz, 1H), 7.63 (s, 1H), 7.34 (d, J=8.5 Hz, 1H), 7.27-7.02 (m, 12H), 4.57-4.46 (m, 2H), 4.18 (dd, J=13.2, 8.3 Hz, 1H), 3.85 (s, 3H), 3.72 (q, J=7.1 Hz, 1H), 2.98 (dd, J=13.8, 3.9 Hz, 2H), 2.82-2.67 (m, 4H), 1.79-1.66 (m, 1H), 1.66-1.46 (m, 3H), 1.33 (dd, J=15.1, 7.7 Hz, 2H), 1.20 (d, J=7.0 Hz, 3H); ¹³C NMR (101 MHz, DMSO-d₆) δ 173.29, 173.23, 170.93, 170.86, 156.95, 137.87, 137.38, 136.91, 133.10, 129.25 (2C), 129.15 (2C), 129.12, 128.33, 127.94 (2C), 127.90 (2C), 126.59, 126.49, 126.16 (2C), 125.38, 118.43, 105.63, 55.14, 53.65, 53.53, 51.69, 44.74, 38.61, 37.55, 37.51, 30.48, 26.58, 22.32, 18.63.

### Naproxen-L-Phe-L-Phe-L-Lys-L-Tyr (L-4)

¹H NMR (400 MHz, DMSO-d₆) δ 9.22 (s, 1H), 8.13-7.99 (m, 4H), 7.73 (d, J=9.0 Hz, 1H), 7.69 (d, J=8.5 Hz, 1H), 7.63 (s, 1H), 7.33 (d, J=8.5 Hz, 1H), 7.28-6.96 (m, 14H), 6.65 (d, J=8.1 Hz, 2H), 4.50 (dd, J=21.3, 12.7 Hz, 2H), 4.39-4.25 (m, 2H), 3.85 (s, 3H), 3.72 (d, J=6.8 Hz, 1H), 3.02-2.89 (m, 3H), 2.86-2.64 (m, 5H), 1.61 (s, 1H), 1.55-1.43 (m, 3H), 1.26 (s, 2H), 1.19 (d, J=7.0 Hz, 3H); ¹³C NMR (101 MHz, DMSO-d₆) δ 173.18, 172.87, 171.10, 170.95, 170.50, 156.93, 155.91, 137.82, 137.40, 136.89, 133.08, 130.01 (2C), 129.25 (2C), 129.09 (3C), 128.31, 127.90 (2C), 127.89 (2C), 127.44, 126.58, 126.48, 126.14, 126.11, 125.35, 118.43, 114.95 (2C), 105.62, 55.14, 53.89, 53.59 (2C), 52.09, 44.73, 38.64, 37.55, 37.40, 35.97, 31.62, 26.63, 21.92, 18.62.

COX Inhibitor Screening Assay.

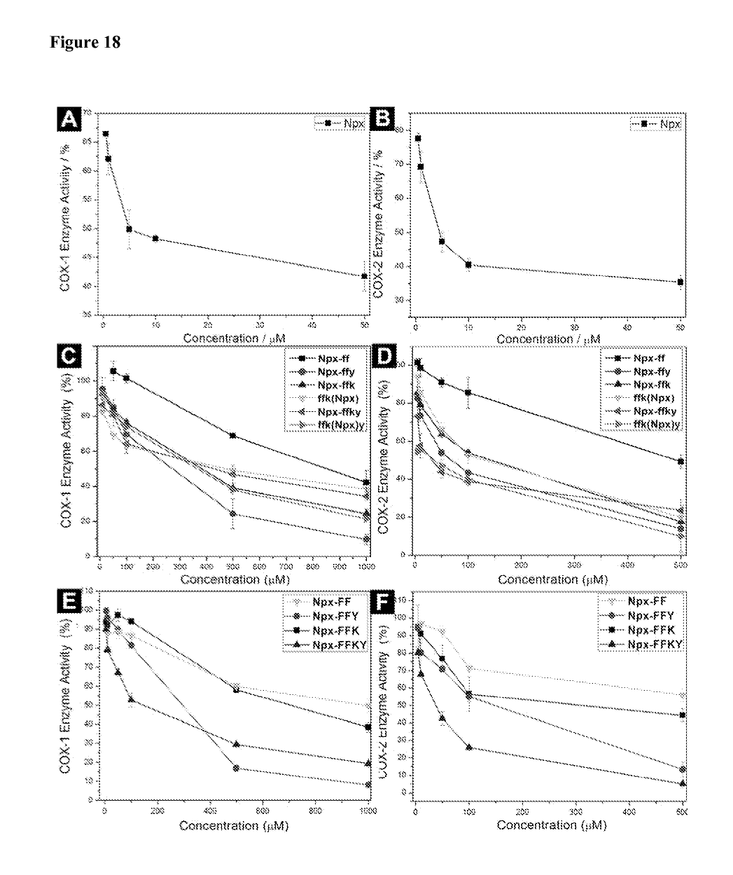

To study the drug efficacies of NSAID containing hydrogelators, here we used ‘COX Fluorescent Inhibitor Screening Assay Kit’ (700100; Cayman Chemical) to do in vitro inhibition assays for Npx and Npx containing hydrogelors 1, 2, 3, 4, 5, and 6. Each compound was tested by COX-1 (ovine) enzyme and COX-2 (human recombinant) enzyme separately in 96 well black assay plates. By utilizing the peroxidase component of COXs, we monitored the reaction between PGG₂ and ADHP (10-acetyl-3,7-dihydroxyphenoxazine), which produced highly fluorescent compound resorufin. This resorufin can be easily analyzed by multimode detector with an excitation wavelength of 530-540 nm and an emission wavelength of 585-595 nm. All the compounds are assayed in triplicate. After the combination of 10 μl of inhibitors, 10 μl of Heme solution, 10 μL of fluorometric substrate, 10 μl of enzyme (either COX-1 or COX-2), and 150 μl of assay buffer, we quickly add 10 μl of arachidonic acid solution to initiate the reaction. Then we read the plate exactly after two minutes incubation at room temperature. In this experiment, we also measure the blank data without enzyme and inhibitors, and the control data without inhibitors. The IC₅₀ values of these hydrogelators were read from their activity curves (FIG. 18), which were measured with 5 different concentrations of these hydrogelators (dissolved in DMSO).

Cytotoxicity.

The HeLa cells in good condition were seeded into 96-well plate (2×10⁵ cells/well) in 100 μL of MEM medium with 10% FBS. With 12 hours of incubation at 37° C. and 5% CO₂, the HeLa cells were attached to bottom of 96-well plate. Then the medium was replaced by another 100 μL of growth medium that contained serial diluents of our compounds and the cells were incubated at 37° C. and 5% CO₂ for additional 72 hours. The compounds were stocked at 10 μM in DMSO, followed by further dilutions with MEM medium. All of these serial diluents were adjusted to contain 0.5% of DMSO. To measure the proliferation of HeLa cell for 3 days, 10 μL of (3-(4,5-Dimethylthiazol-2-yl)-2,5-diphenyltetrazolium bromide (MTT, 0.5 mg/mL) was added every 24 hours, followed by adding 100 μL of 0.1% sodium dodecyl sulfate (SDS) 4 hours later. With medium as blank and untreated HeLa cell as control, we measure each concentration of these compounds in triplicate. Since the mitochondrial reductase in living cells reduced MTT to purple fomazan, the absorbance at 595 nm of the whole solution was finally measured by DTX 880

Multimode Detector.

With the measurement of 5 different concentrations of these hydrogelators, their IC₅₀ values were read from the activity curves for day 3 (FIG. 20). TEM sample preparation. The TEM images we reported in this paper were taken by negative staining technique. Carbon coated grids (400 mesh copper grids that had been coated with continuous thick carbon film ˜35 nm) were first glowed discharge just before use to increase their hydrophilicity. After sample solution placed onto the grid (3 μL, sufficient to cover the grid surface), the grid was rinsed by DI H₂O for 3 times (let the grid touch the water drop, with the sample-loaded surface facing the parafilm, then tilt the grid and gently absorb water from the edge of the grid using a filter paper sliver). Immediately after rinsing, the grid was stained by UA stain solution (2.0% (w/v) uranyl acetate) for 3 times (let the grid touch the stain solution drop, with the sample-loaded surface facing the parafilm, then tilt the grid and gently absorb the stain solution from the edge of the grid using a filter paper sliver.) Then we allow the grid to dry in air and examine the grid as soon as possible.

General Procedure for Anti-HIV Drug Release from Hydrogels.

To illustrate the in vitro release profile of hydrogelators, we prepared 100 μL of the hydrogels formed by 1 (0.8 wt %, pH 4.0), 2 (0.8 wt %, pH 7.6), 3 (0.8 wt %, pH 7.6), 4 (0.8 wt %, pH 7.6), 5 (0.8 wt %, pH 7.0), and 6 (0.8 wt %, pH 7.0). With the addition of PBS buffer (100 μL, pH 7.4) onto the surface of hydrogels, the gels were incubated at 37° C. for 24 hours. The release solutions (100 μL) were taken and refreshed at 0 h, 2 h, 4 h, 8 h, 12 h, and 24 h, which were detected by analytical HPLC at 276 nm for the quantities of released Npx containing hydrogelators 1, 2, 3, 4, 5, and 6.

General Procedure for Hydrogel Preparation.

All the compounds were dissolved in de-ionized water. The pH of the solutions were adjusted by 1 M of NaOH and 1 M of HCl and measured by pH paper. Then the hydrogels were formed by changing the solutions to weekly acidic, or by adding enzymes (alkaline phosphatase) in a weakly basic.

Rheological Measurement.

Rheological tests were conducted on TA ARES G2 rheometer with TA Orchestrator Software. 25 mm cone-plate was used during the experiment. 0.2 mL of hydrogel sample was placed on the cone-plate.

### (1) Dynamic Strain Sweep Tests for the Investigation of the System Structures:

The measurement performs at the frequency of 6.28 rad/s (0.1 to 10% strain, frequency=10 rads⁻¹, 10 points per decade). Sweep mode is “log” and temperature was carried out at 25° C. The critical strain (γ_{c}) value was determined from the storage-strain profiles of the hydrogel sample. The strain applied to the hydrogel sample increased from 0.1 to 100% (10 rad/s and 25° C.). Over a certain strain, a drop in the elastic modulus was observed, and the strain amplitude at which storage moduli just begins to decrease by 5% from its maximum value was determined and taken as a measure of the critical strain of the hydrogels, which correspond to the breakdown of the cross-linked network in the hydrogel sample.

### (2) Dynamic Frequency Sweep Tests for the Investigation of the Viscoelastic Properties:

The frequency ranges from 200 rad/s to 0.1 rad/s, depending on the viscoelastic properties of each sample. A suitable strain was used to ensure the linearity of dynamic viscoelasticity.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A hydrogelator of Formula III 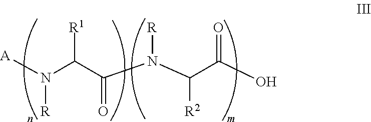
or a pharmaceutically acceptable salt thereof,
wherein, independently for each occurrence,
A is selected from the group consisting of 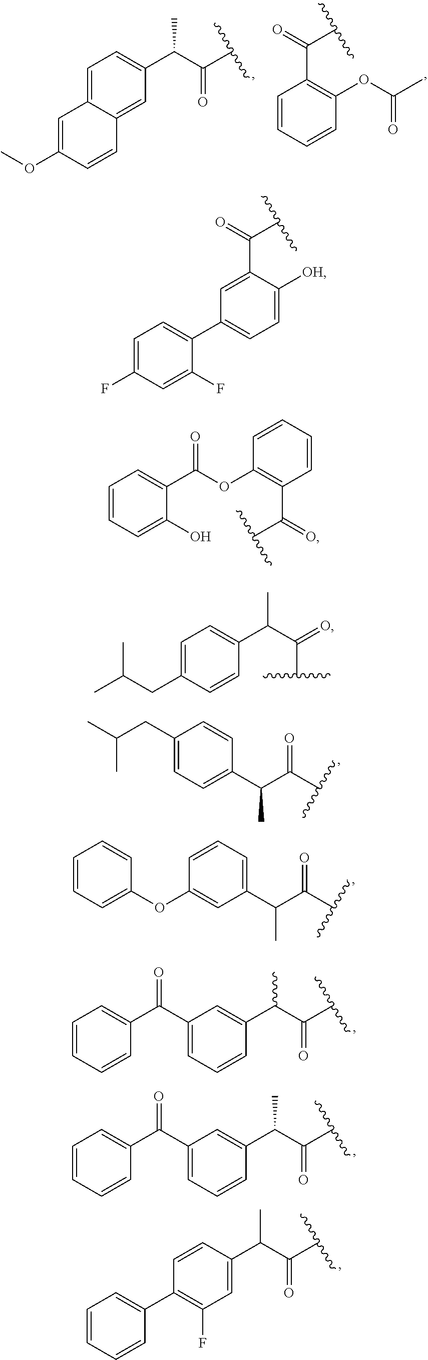 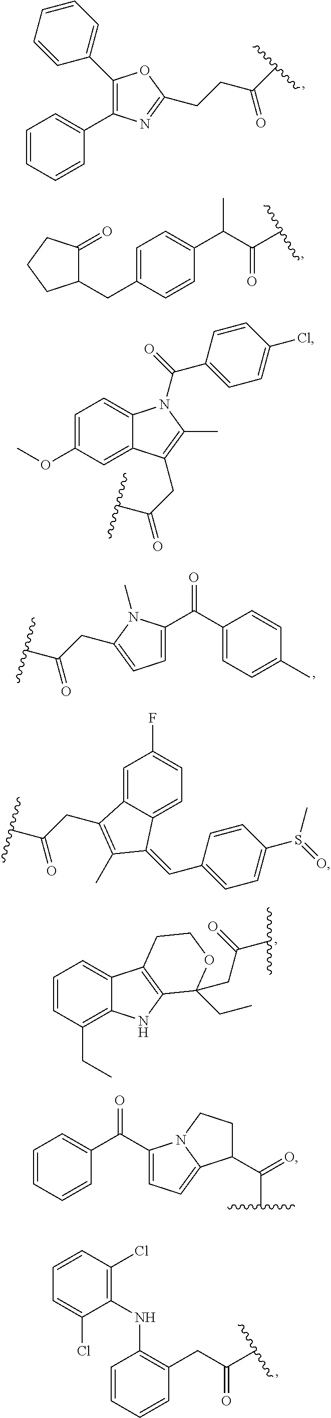 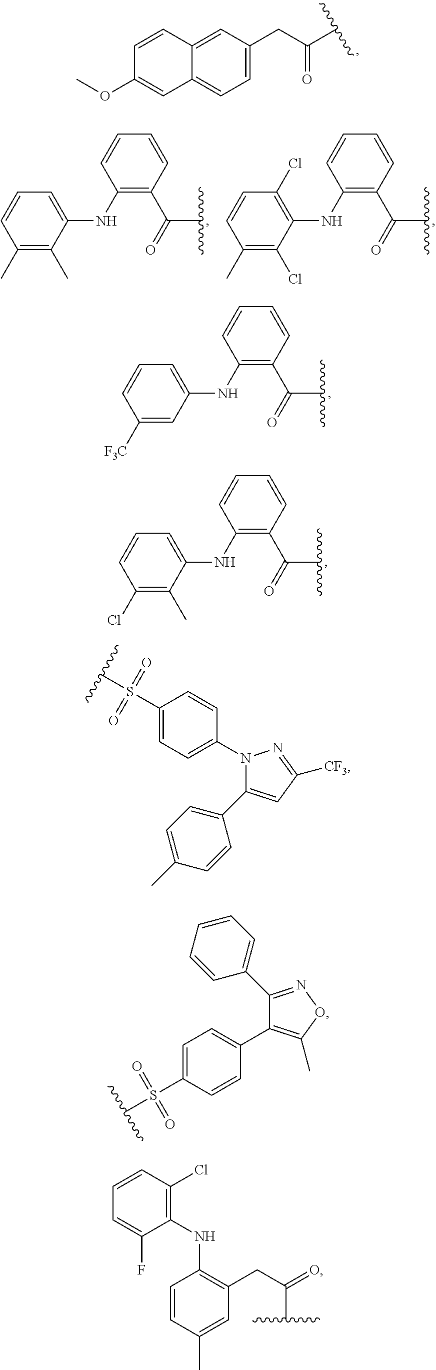 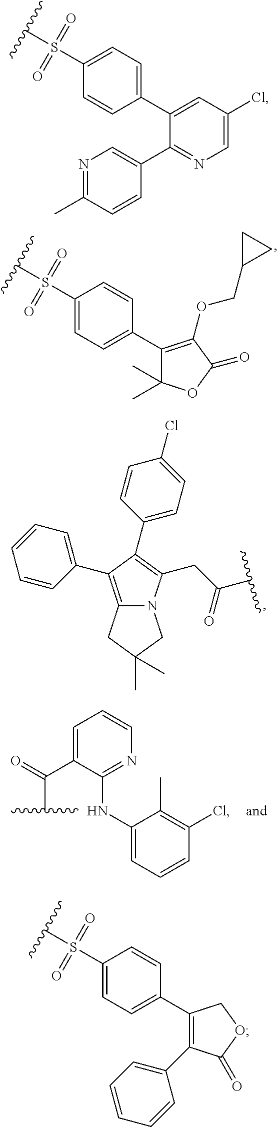
R is H or alkyl;
R¹ is aralkyl, heteroaralkyl, hydroxyaralkyl, or phosphorylated aralkyl;
R² is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, or HS-alkyl;
n is 1, 2, 3, or 4; and
m is 0, 1, 2, 3, or 4.

2. The hydrogelator of claim 1, wherein A is selected from the group consisting of:

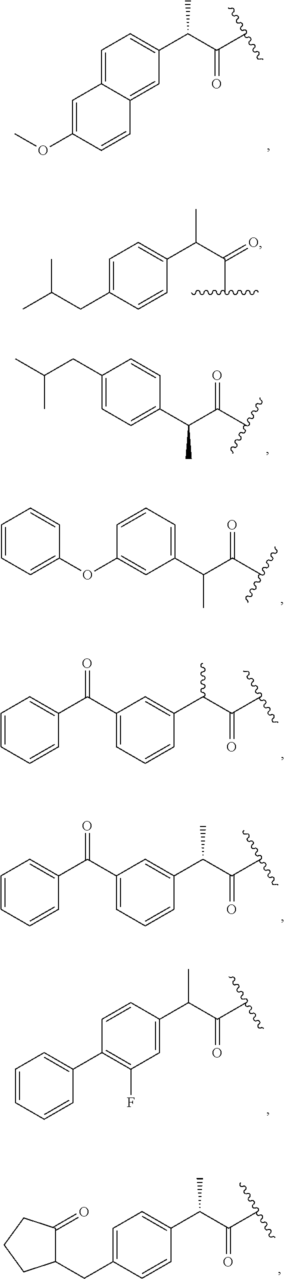

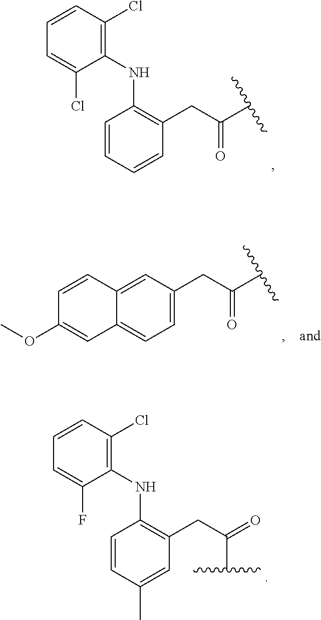 [SMILES: *C(=O)Cc1ccccc1Nc1c(Cl)cccc1Cl ; *C(=O)Cc1ccc2cc(OC)ccc2c1 ; *C(=O)Cc1cc(C)ccc1Nc1c(F)cccc1Cl]

3. (canceled)

4. The hydrogelator of claim 1, wherein R is H.

5. The hydrogelator of claim 1, wherein R¹ is aralkyl or heteroaralkyl.

6. (canceled)

7. The hydrogelator of claim 5, wherein R¹ is benzyl.

8. The hydrogelator of claim 1, wherein R² is aralkyl, hydroxyaralkyl, phosphorylated aralkyl, alkyl, aminoalkyl, or hydroxyalkyl.

9. (canceled)

10. The hydrogelator of claim 8, wherein R² is hydroxybenzyl.

11. (canceled)

12. The hydrogelator of claim 8, wherein R² is phosphorylated benzyl.

13. (canceled)

14. The hydrogelator of claim 8, wherein R² is aminobutyl.

15. The hydrogelator of claim 1, wherein n is 1, 2, or 3.

16. (canceled)

17. The hydrogelator of claim 1, wherein m is 0, 1, or 2.

18-19. (canceled)

20. The hydrogelator of claim 1, wherein each amino acid residue is in the D-configuration.

21. A hydrogelator of Formula IV 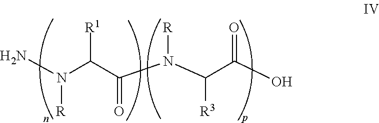
or a pharmaceutically acceptable salt thereof,
wherein, independently for each occurrence,
R is H or alkyl;
R¹ is aralkyl, heteroaralkyl, hydroxyaralkyl, or phosphorylated aralkyl;
R³ is H, aralkyl, heteroaralkyl, hydroxyaralkyl, phosphorylated aralkyl, alkyl, aminoalkyl, HO₂C-alkyl, hydroxyalkyl, H₂NC(═O)-alkyl, HS-alkyl, or A-NR-alkyl, provided at least one instance of R³ is A-NR-alkyl;
n is 1, 2, 3, or 4;
p is 1, 2, 3, or 4; and
A is selected from the group consisting of

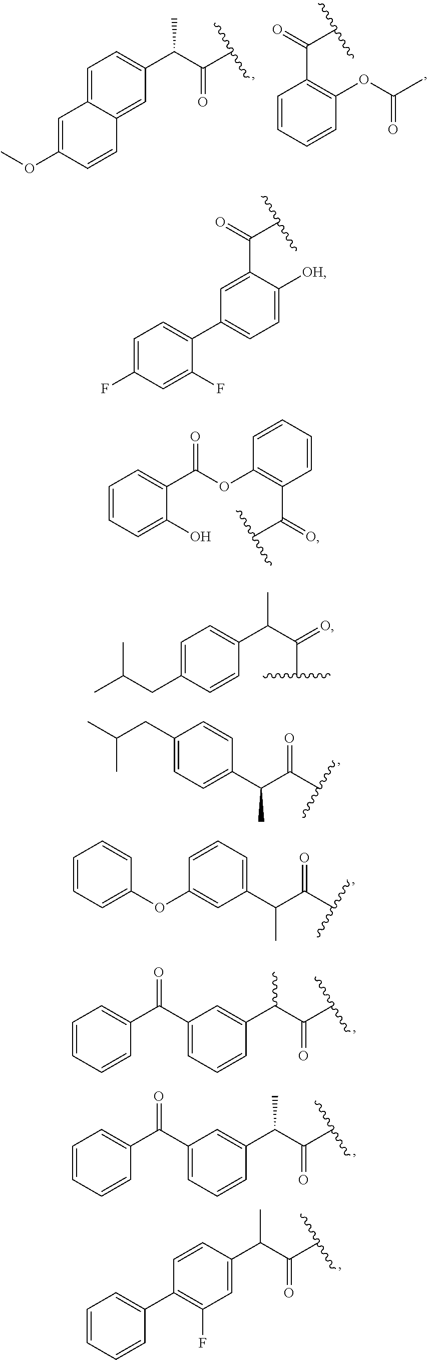

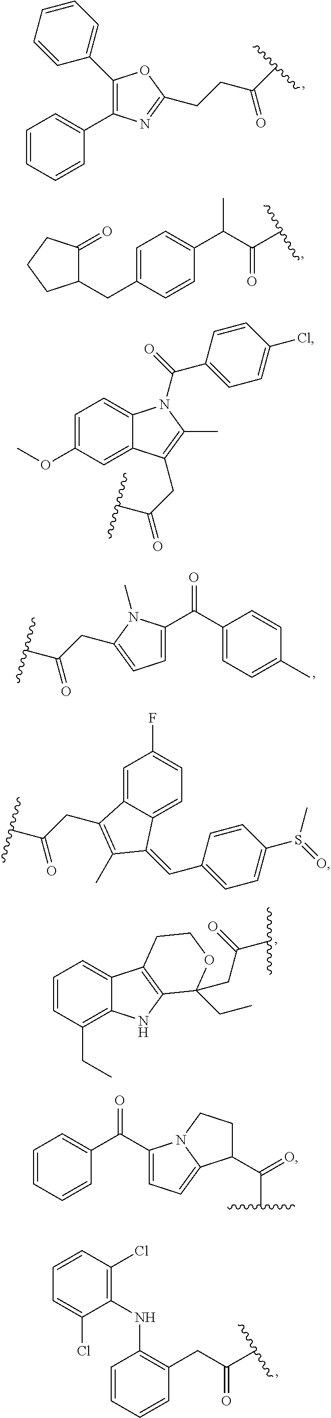

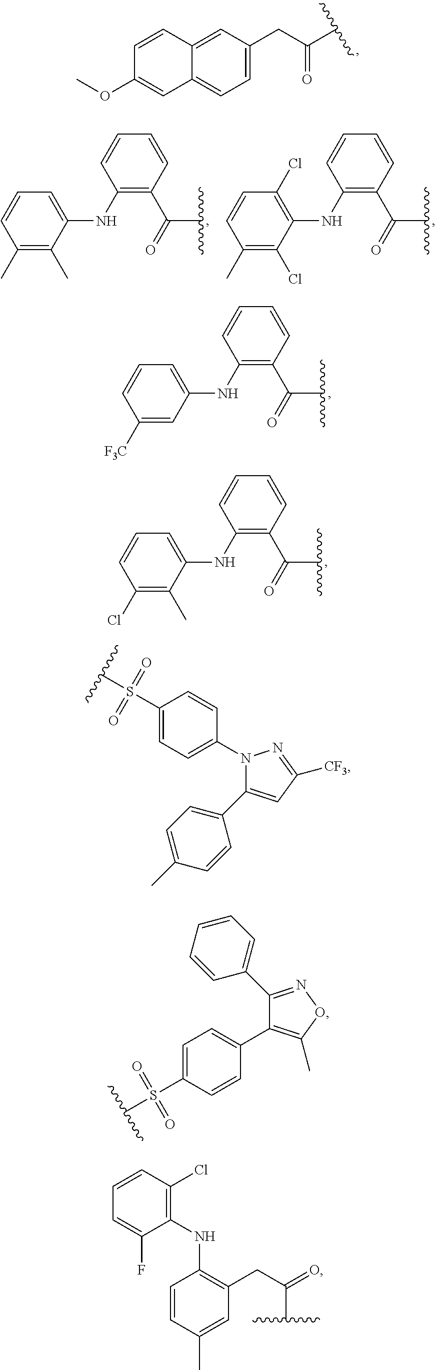

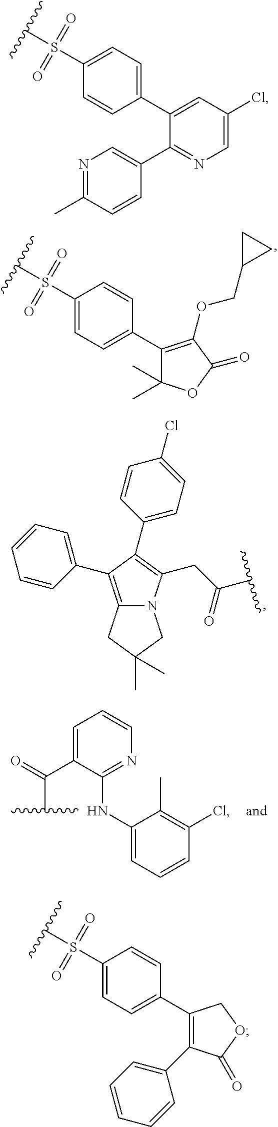

22. The hydrogelator of claim 21, wherein A is selected from the group consisting of:

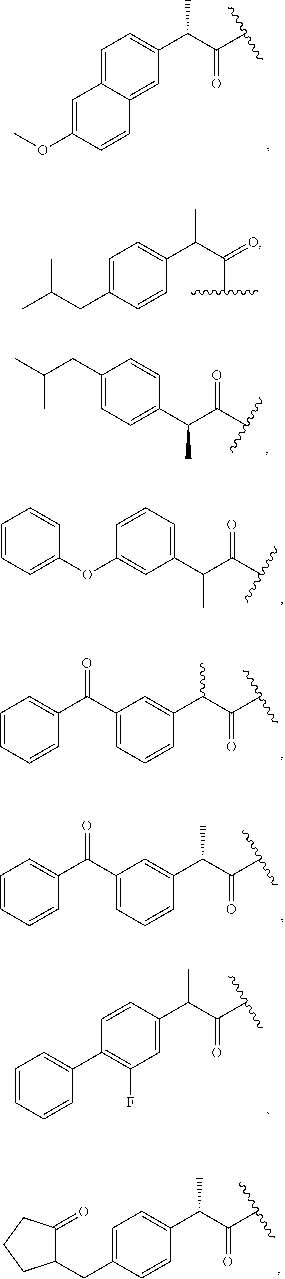

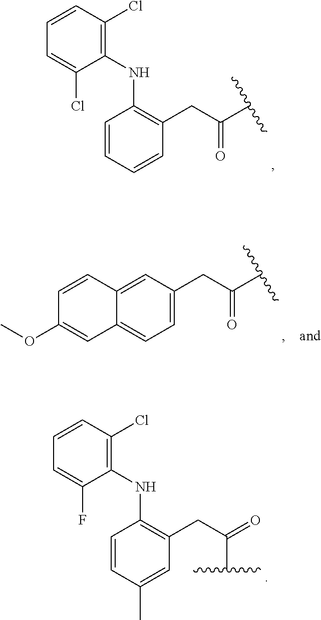 [SMILES: *C(=O)Cc1ccccc1Nc1c(Cl)cccc1Cl ; *C(=O)Cc1ccc2cc(OC)ccc2c1 ; *C(=O)Cc1cc(C)ccc1Nc1c(F)cccc1Cl]

23. (canceled)

24. The hydrogelator of claim 21, wherein R is H.

25. The hydrogelator of claim 21, wherein R¹ is aralkyl or heteroaralkyl.

26. (canceled)

27. The hydrogelator of claim 25, wherein R¹ is benzyl.

28. The hydrogelator of claim 21, wherein n is 1, 2, or 3.

29. (canceled)

30. The hydrogelator of claim 21, wherein R³ is A-NR-alkyl.

31-32. (canceled)

33. The hydrogelator of claim 30, wherein R³ is A-NH-butyl.

34. The hydrogelator of claim 21, wherein p is 1, 2, or 3.

35-36. (canceled)

37. The hydrogelator of claim 21, wherein each amino acid residue is in the D-configuration.

38. A hydrogelator selected from the group consisting of: 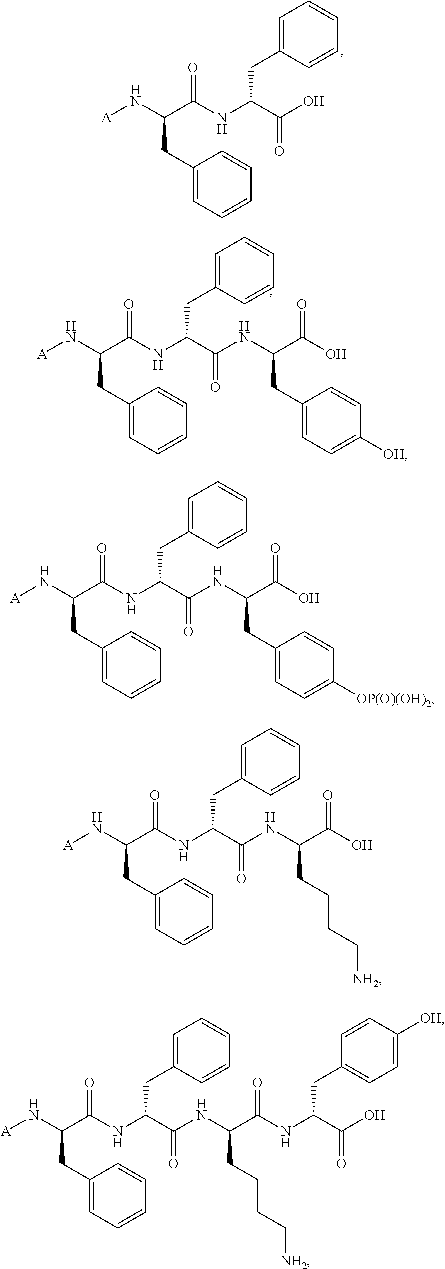 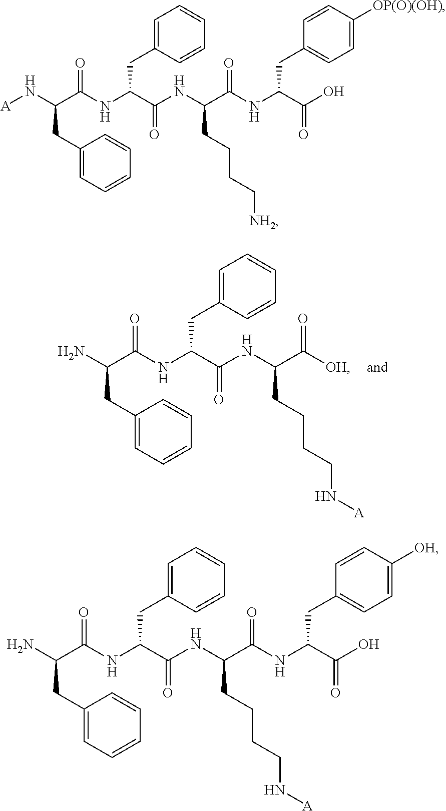
wherein A is selected from the group consisting of 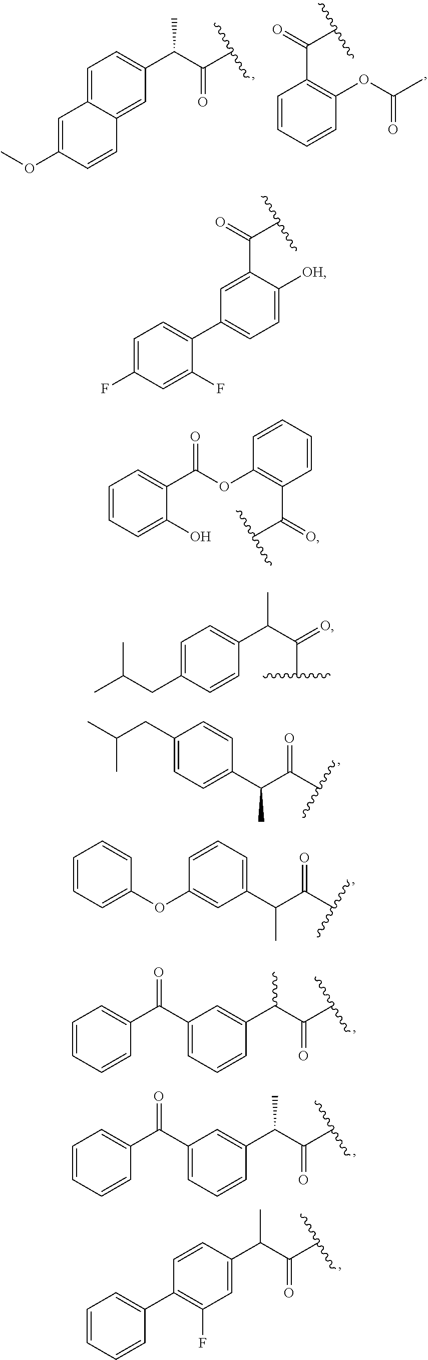 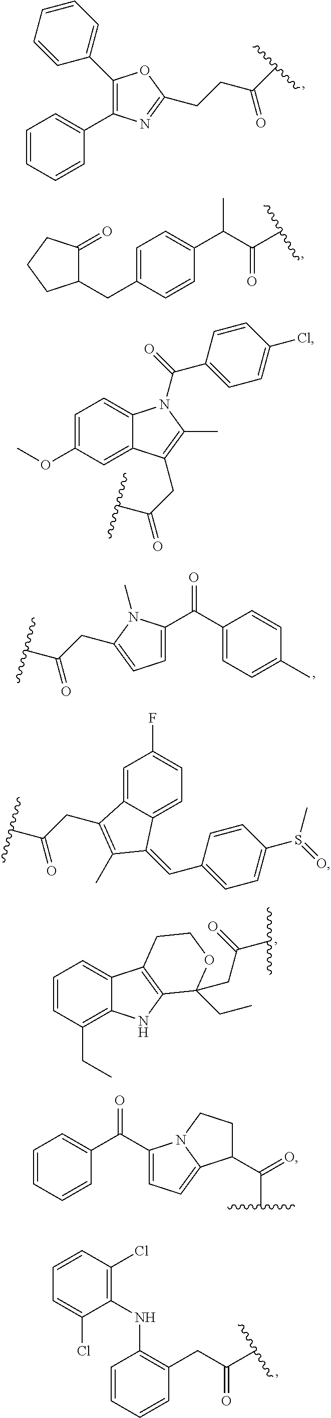 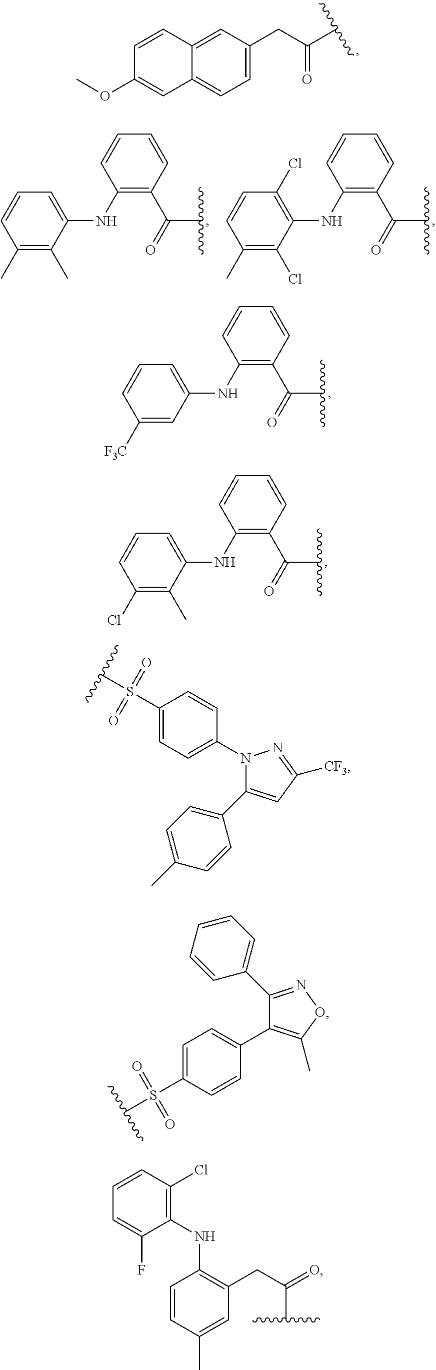 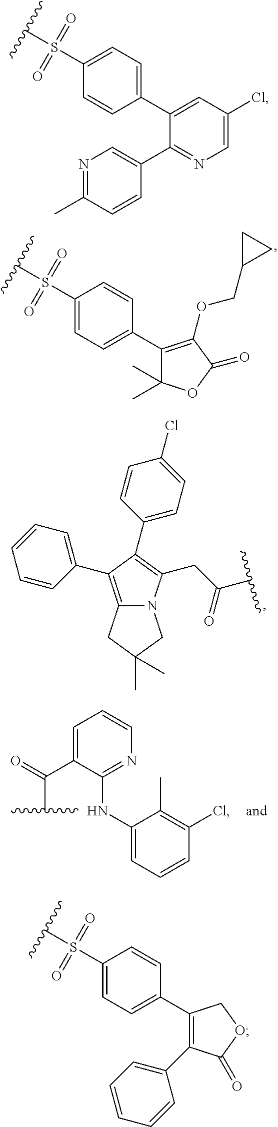
or a pharmaceutically acceptable salt thereof.

39. A hydrogelator of claim 38, wherein the hydrogelator is selected from the group consisting of: 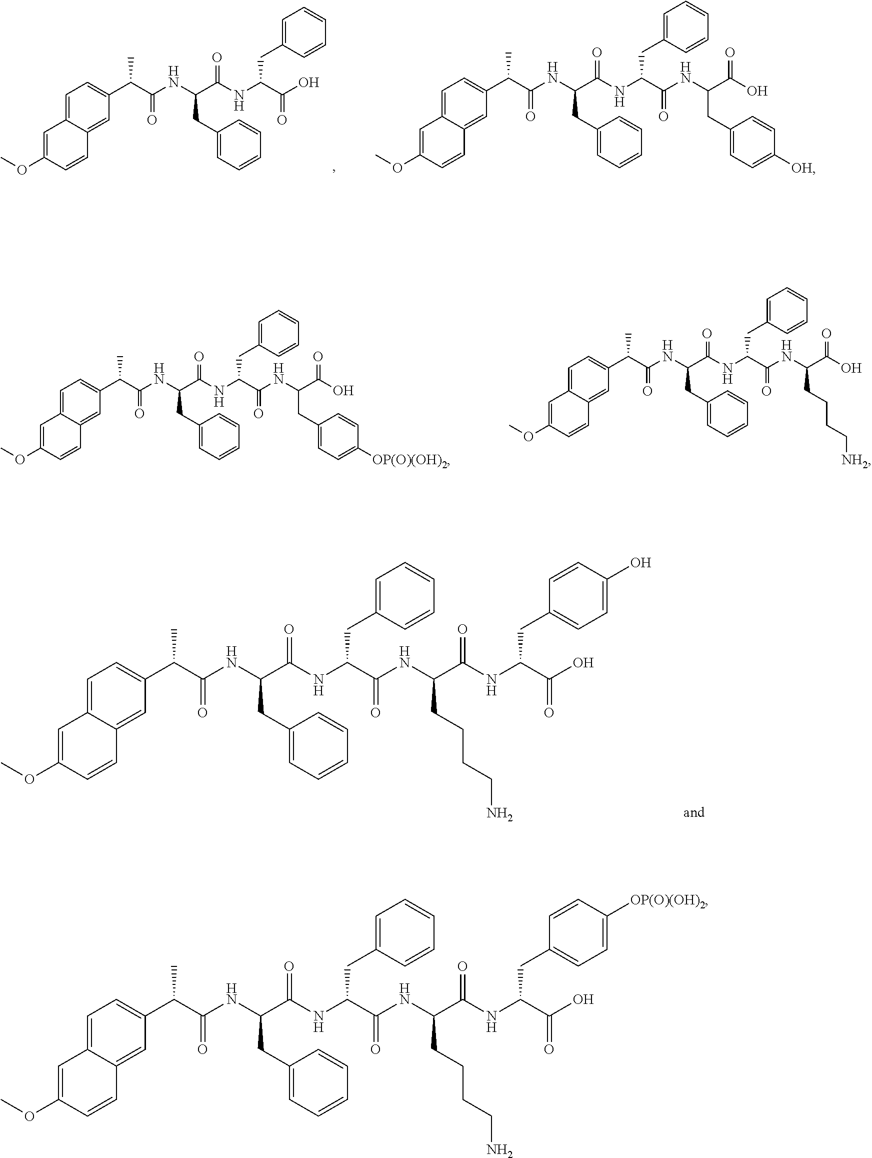
or a pharmaceutically acceptable salt thereof.

40. A hydrogelator of claim 38, wherein the hydrogelator is selected from the group consisting of: 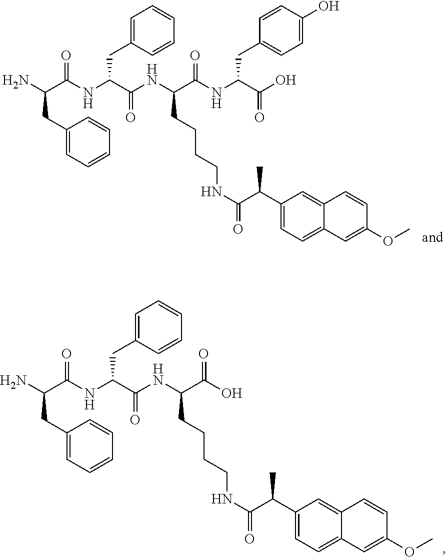 [SMILES: COc1ccc2cc([C@H](C)C(=O)NCCCC[C@@H](NC(=O)[C@@H](Cc3ccccc3)NC(=O)[C@H](N)Cc3ccccc3)C(=O)N[C@H](Cc3ccc(O)cc3)C(=O)O)ccc2c1 ; COc1ccc2cc([C@H](C)C(=O)NCCCC[C@@H](NC(=O)[C@@H](Cc3ccccc3)NC(=O)[C@H](N)Cc3ccccc3)C(=O)O)ccc2c1]
or a pharmaceutically acceptable salt thereof.

41. A supramolecular structure consisting essentially of a plurality of hydrogelators of claim 1.

42. The supramolecular structure of claim 41, wherein the supramolecular structure is in the form of nanofibers.

43-45. (canceled)

46. The supramolecular structure of claim 42, wherein the nanofibers form networks or bundles.

47-48. (canceled)

49. A hydrogel, consisting essentially of a plurality of hydrogelators of claim 1; and water.

50. The hydrogel of claim 49, wherein the hydrogelator is present in an amount of about 0.2% to about 4% by weight.

51. The hydrogel of claim 49 or 50, wherein the hydrogel has a critical strain value of about 0.2% to about 10.0%.

52. The hydrogel of claim 49, wherein the hydrogel has a storage modulus of about 75 Pa to about 70 KPa.

53. A method of treating an inflammatory condition, comprising:
administering to a subject in need thereof a therapeutically effective amount of a hydrogel of claim 49.

54. The method of claim 53, wherein the hydrogel is administered topically.

55. The method of claim 53, wherein the hydrogel is administered to the skin of the subject in need thereof.

56. The method of claim 53, wherein the hydrogel is in the form of a lotion, cream, or gel.

57. The method of claim 53, wherein the inflammatory condition is selected from the group consisting of osteoarthritis, rheumatoid arthritis, psoriatic arthritis, gout, tendinitis, bursitis, and ankylosing spondylitis.

58. A supramolecular structure consisting essentially of a plurality of hydrogelators of claim 21.

59. A hydrogel, consisting essentially of a plurality of hydrogelators of claim 21; and water.

60. A method of treating an inflammatory condition, comprising:
administering to a subject in need thereof a therapeutically effective amount of a hydrogel of claim 59.
